SEQUENCE LISTING (4 sequences)

SEQ ID NO: 1 - DNA, 20 bp, synthetic construct
  source 1..20: mol_type = other DNA
gggaaacgcctggtatcttt

SEQ ID NO: 2 - DNA, 20 bp, synthetic construct
  source 1..20: mol_type = other DNA
tggaaagtccctattggcgt

SEQ ID NO: 3 - DNA, 46 bp, synthetic construct
  source 1..46: mol_type = other DNA
gaaaaaggccggccaggcaaaaaagaaaaagttattcggcagtgga

SEQ ID NO: 4 - DNA, 45 bp, synthetic construct
  source 1..45: mol_type = other DNA
tagttagaattcttacttgtacagctcgtccatgccgagagtgat

(19) United States
(12) Patent Application Publication
(10) Pub. No.: US 2026/0224754 A1
(43) Pub. Date: Aug. 6, 2026

(54) ORAL FORMULATION TO ENSURE BIOAVAILABILITY OF PEPTIDE, PROTEIN, AND NUCLEIC ACID THERAPEUTICS

(71) Applicant: THE TRUSTEES OF COLUMBIA UNIVERSITY IN THE CITY OF NEW YORK, New York, NY (US)

(72) Inventors: Kam W. LEONG, New York, NY (US); Stavroula KOUSTENI, New York, NY (US); Yuefei ZHU, New York, NY (US); Jessica DEANGELIS, New York, NY (US); Ioanna MOSIALOU, Astoria, NY (US); Gerald KARSENTY,, New York, NY (US); Morgan Luben ZVEZDOV, New York, NY (US); Chai Hoon QUEK, New York, NY (US); Huiyi LIANG, New York, NY (US); Suwan DING, New York, NY (US); Veronica FARAG, New York, NY (US)

(21) Appl. No.: 19/645,013

(22) Filed: Apr. 10, 2026

Related U.S. Application Data
(63) Continuation of application No. PCT/US24/51460, filed on Oct. 15, 2024.
(60) Provisional application No. 63/589,913, filed on Oct. 12, 2023.
(60) Provisional application No. 63/651,294, filed on May 23, 2024.
(60) Provisional application No. 63/651,819, filed on May 24, 2024.
(60) Provisional application No. 63/658,809, filed on Jun. 11, 2024.

(51) Int. Cl. A61K 48/00; A61K 9/48; A61K 9/50; A61K 38/17

(52) U.S. Cl. CPC A61K 48/0075; A61K 9/4833; A61K 9/5031; A61K 9/5036; A61K 9/5068; A61K 38/1709

(57) ABSTRACT

The disclosure concerns oral formulations that enable delivery of therapeutic agents that are sensitive to stomach pH and/or have reduced ability to be absorbed. In one aspect, the oral formulation comprises: (a) phenyl boric acid (PBA)-functionalized chitosan grafted with branched polyethyleneimine (PEI) and (b) a therapeutic agent, wherein the therapeutic agent is encapsulated by the PBA-functionalized chitosan grafted with a branched PEI. In other aspects, the oral formulation comprises: (a) chitosan grafted with branched polyethyleneimine (PEI), (b) a therapeutic agent, wherein the therapeutic agent is encapsulated by the chitosan grafted with a branched PEI, and optionally (c) a eukaryotic cell membrane fragment. The disclosure also concerns methods of making and administering such oral formulations.

### RELATED APPLICATIONS

This application is a continuation application of International Application No. PCT/US24/51460, filed Oct. 15, 2024, titled “ORAL FORMULATION TO ENSURE BIOAVAILABILITY OF PEPTIDE, PROTEIN, AND NUCLEIC ACID THERAPEUTICS”, which claims priority to and the benefit of U.S. provisional patent application 63/589,913, filed Oct. 12, 2023, titled “ORAL FORMULATION TO ENSURE BIOAVAILABILITY OF PEPTIDE AND PROTEIN DRUGS,” U.S. provisional patent application 63/651,294, filed May 23, 2024, titled “TARGETED GENE EDITING IN MESENTERIC LYMPH NODES BY ORAL NON-VIRAL NANOPARTICLES,” U.S. provisional patent application 63/651,819, filed May 24, 2024, titled “TAILORED PHENYLBORONIC ACID-FUNCTIONALIZED CHITOSAN COMPLEXES FOR SIRNA DELIVERY,” and U.S. provisional patent application 63/658,809, filed Jun. 11, 2024, titled “ORAL BIOAVAILABLE OSTEOCALCIN DRUG FOR TREATMENT OF SARCOPENIA AND REVERSAL OR AGE-RELATED MEMORY LOSS,” the entirety of the disclosures of which are hereby incorporated by this reference.

### STATEMENT REGARDING FEDERALLY SPONSORED RESEARCH OR DEVELOPMENT

This invention was made with government support under AG032959, AR054447, AR073180, and DK007328 awarded by the National Institutes of Health. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 4,527 byte XML file named “SeqList_182US-PAT2” created on Apr. 9, 2026.

### TECHNICAL FIELD

This document relates to oral delivery of therapeutic agents with novel formulations and their methods of production and use.

### BACKGROUND

Delivery of therapeutic agents orally can be challenging because of the wide range of pHs found within the stomach and the intestine. In addition, the therapeutic agent needs to be able be delivered to the desired location within a patient's body, which is limited with oral administration.

Peptide, protein, or DNA therapeutics are typically administered intravenously so that the therapeutic agent can be delivered to the desired location without being degraded or destroyed in the conditions of the stomach. But the invasiveness of conventional methods of administering peptide, protein, or DNA therapeutics limit the use of these therapeutic agents. Thus, oral formulation that successfully administer peptide, protein, or DNA therapeutics are needed.

Efficient delivery also limits the potential usefulness of gene editing with the clustered regularly interspaced short palindromic repeats (CRISPR)/CRISPR-associated protein 9 (Cas9) system. Despite significant efforts to improve the delivery of CRISPR elements to various tissue targets for somatic genome editing, none have demonstrated the feasibility of oral CRISPR delivery. A viable, orally administered, nonviral gene editing system may open up new exciting opportunities to address the needs of both local and systemic gene editing in a patient-compliant, noninvasive, and repeatable manner. There is a need for oral delivery strategies to accomplish these requirements.

### SUMMARY

In some aspects, the disclosure concerns oral formulations comprising: (a) a polymer comprising phenyl boric acid (PBA)-functionalized chitosan grafted with branched polyethyleneimine (PEI); and (b) a therapeutic agent, wherein the therapeutic agent is encapsulated by the polymer. In some aspects, the PBA-functionalized chitosan grafted with PEI is a block copolymer. In some aspects, the PBA is 4-Carboxy-3-fluorophenylboronic acid. In some embodiments, such oral formulations further comprise eukaryotic cell membrane fragment, for example yeast membrane fragment. The eukaryotic cell membrane fragment coats the polymer encapsulating the therapeutic agent.

In other aspects, the disclosure concerns oral formulations comprising: (a) a polymer comprising chitosan grafted with branched PEI; (b) a therapeutic agent, wherein the therapeutic agent is encapsulated by the polymer; and optionally (c) eukaryotic cell membrane fragment that coats the polymer encapsulating the therapeutic agent. In some embodiments, the chitosan grafted with branched PEI is a PBA-functionalized chitosan grafted with PEI. In some aspects, the PBA is 4-Carboxy-3-fluorophenylboronic acid.

In some aspects, the chitosan in the oral formulations has a molecular weight of about 1,000 Da to about 40,000 Da or has a molecular weight of about 15,000 Da. In some aspects, the branched PEI in the oral formulations has a molecular weight of about 600 Da to about 10,000 Da or has a molecular weight of about 800 Da to about 2,000 Da.

In certain embodiments, the polymer in the oral formulation is of the formula CS-PEI_{X%}(PBA)_{Y%} where CS is a chitosan residue, PBA is a phenyl boric acid residue, and PEI is a branched polyethyleneimine polymer residue, and where X % is the PEI grafting ratio and Y % is the PBA introduction ratio, and wherein X % is 35 to 75%, Y % is 5 to 59%.

In some aspects, amount of therapeutic agent in the oral formulation is 1.79 to 21.4% relative to the amount of polymer. In some aspects, the loading capacity of the polymer is around 17.8% to 22%.

In some aspects, the therapeutic agent is a protein or peptide therapeutic. For example, the protein or peptide therapeutic is Lipocalin 2 or osteocalcin. In other aspects, the therapeutic agent delivers gene therapy. Thus, in some implementations, therapeutic agent is a plasmid. In some embodiments, the therapeutic agent includes a CRISPR/Cas system. In other embodiments, the therapeutic agent is siRNA.

In certain aspects, the disclosure concerns methods of producing an orally bioavailable form of Lipocalin 2 (LCN2) comprising encapsulating LCN2 with a polymer comprising PBA-functionalized chitosan grafted with a branched PEI.

In other aspects, the disclosure concerns methods of reducing weight in a patient comprising orally administering to the patient a formulation comprising: (a) a polymer comprising PBA-functionalized chitosan grafted with branched PEI and (b) LCN2, wherein the LCN2 is encapsulated by the PBA-functionalized chitosan grafted with branched PEI. In some embodiments, the subject is obese.

In some aspects, the techniques described herein relate to a method of producing an orally administered gene therapy product. In some embodiments, the method comprises encapsulating the gene therapy product with a polymer comprising PBA-functionalized chitosan grafted with a branched PEI to produce an encapsulated gene therapy product. In some implementations, the method further comprises coating the encapsulated gene therapy product with a eukaryotic cell membrane fragment. In some aspects, the polymer is formed by contacting a branched PEI chitosan polymer with a boric acid (for example 2-fluro-4-carboxyphenyl boric acid) in a solvent. In other embodiments, the method comprises encapsulating the gene therapy product with a polymer comprising chitosan grafted with a branched PEI to produce an encapsulated gene therapy product and coating the encapsulated gene therapy product with a eukaryotic cell membrane fragment. In some implementations, the polymer that encapsulates the gene therapy product is PBA-functionalized chitosan grafted with a branched PEI. The gene therapy product may be a plasmid or comprises siRNA or a CRISPR/Cas system.

Some embodiments of the disclosed methods use chitosan having a molecular weight of about 1,000 Da to about 40,000 Da. In some aspects, the branched PEI has a molecular weight of about 600 Da to about 10000 Da. In certain embodiments, the branched PEI has a molecular weight of about 600 Da to about 2200 Da. In certain implementations of the disclosed method, the polymer is of the formula CS-PEI_{X%}(PBA)_{Y%} where CS is a chitosan residue, PBA is a phenyl boric acid residue, and PEI is a branched polyethyleneimine polymer residue, and where X % is the PEI grafting ratio and X % is the PBA introduction ratio, and wherein x % is 35 to 75%, Y % is 5 to 59%.

The foregoing and other aspects, features, and advantages will be apparent from the DESCRIPTION and DRAWINGS, and from the CLAIMS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

FIG. 1 shows the equilibrium of phenylboronic acid (PBA) in aqueous solution.

FIG. 2A-2B depict a representation of the designed polymers comprising phenyl boric acid (PBA)-functionalized chitosan grafted with a branched polyethyleneimine (PEI) (CS-PEI (PBA)) nanoparticles. In FIG. 2A, a synthetic scheme of CS-PEI (PBA) is presented. In FIG. 2B, the library design by altering the PEI and PBA ratios is shown.

FIG. 3 is a representation of cytotoxicity screening of the established CS-PEI (PBA) library against the hematopoietic stem progenitor cells.

FIG. 4 depicts a representation of imaging illustrating a confocal laser scanning microscopy (CLSM) image of Cy5-labeled BSA-loaded nanoparticles. In the figure, blue represents cell nuclei, green represents late endosomes/lysosomes, and magenta represents Cy5-BSA.

FIG. 5 shows a representative 1H-NMR spectrum of (top)CS-PEI73% and (bottom) CS-PEI73% (FPBA) 57% in D20 at room temperature.

FIG. 6 presents a schematic illustration of the in vivo behavior of CS-PEI (FPBA) polyplexes after oral administration.

FIG. 7 presents a schematic illustration of the in vivo behavior of Lignocal™ (CPB@LCN2) after oral administration.

FIGS. 8A-8C depict a representation of the designed polymers comprising phenyl boric acid (PBA)-functionalized chitosan grafted with a branched polyethyleneimine (PEI) (CS-PEI (PBA)) nanoparticles for oral LCN2 delivery. In FIG. 8A, the size and zeta potential of CS-PEI (PBA) nanoparticles encapsulated with LCN2 protein is illustrated. FIGS. 8B and 8C show encapsulation efficiency, loading efficiency, and drug release profile of CS-PEI (PBA) nanoparticles.

FIGS. 9A-9D present the in vivo animal study using the LCN2−/− mice. FIG. 9A illustrates blood serum concentration of LCN2 in the LCN2−/− mice. FIG. 9B shows blood serum concentration of the knockout mice at the time of organ collection. FIG. 9C shows in vivo biodistribution of LCN2 in different organs at 50 hours. FIG. 9D presents a comparison of body weight changes in LCN2−/− mice after oral gavage compared to the pure polymer oral gavage group.

FIG. 10 depicts a schematic illustration of the in vivo behavior of OsteoPulse™ (CPB@OCN) after oral administration.

FIGS. 11A-11C present a representation of the designed polymers comprising phenyl boric acid (PBA)-functionalized chitosan grafted with a branched polyethyleneimine (PEI) (CS-PEI (PBA)) nanoparticles for oral OCN delivery. In FIGS. 11A and 11B show the size and zeta potential of CS-PEI (PBA) nanoparticles encapsulated with OCN protein is illustrated, and the size and zeta potential of CS-PEI (PBA) nanoparticles co-loaded with BSA and OCN protein. FIG. 11C shows the polymeric nanoparticle loaded with BSA protein and its corresponding drug release profile.

FIGS. 12A and 12B present a schematic representation of the in vitro myoblast functional assay and the quantification of relative IL-6 mRNA expression levels following treatment with CS-PEI (PBA) nanoparticles (CPB).

FIGS. 13A-13C present characterization of C-BX/BY. FIG. 13A shows size (bar; left Y-axis), PDI (black circle; right, black Y-axis), and ζ-potential (gray circle; right, gray Y-axis) of polyplexes measured by DLS measurements. FIG. 13B shows gel electrophoresis after incubating polyplex with dextran sulfate at pH 7.4. FIG. 13C presents FRET efficiency of double-labeled plasmid in polyplexes (n=3).

FIGS. 14A-14F presents in vitro assays of C-B_{X}/B_{Y}. FIG. 14A shows transfection efficiency at 48 h and FIG. 14B shows cellular uptake efficiency at 24 h in HCT116 cells. FIG. 14C presents CLSM images of the HCT116 cells at 24 h after being treated with polyplexes carrying Cy3-labeled plasmids. FIG. 14D shows quantification of colocalization ratio of Cy3-plasmid with late endosomes/lysosomes in 25 individual cells. FIG. 14E presents intracellular FRET efficiency of double-labeled plasmid in HCT116 cells. FIG. 14F shows transfection efficiency in Caco-2 and Caco-2/HT29-MTX coculture (n=3). Each dot in FIGS. 14A, 14B, 14C, and 14F represents a biological replicate, and statistical analysis was carried out using one-way ANOVA with Dunnett's posthoc test. Unpaired two-tailed Student's t test was used for statistical analysis for FIG. 14E. Significance presented as *p<0.05 and **p<0.01.

FIGS. 15A-15E present in vivo optimization and validations of polyplexes. FIG. 15A presents gel electrophoresis of barcode PCR amplicon extracted from the muscularis externa (outer part; top) and intestinal epithelium layer (inner part; bottom) in the small intestine at 24 h post administration. FIG. 15B shows the amount of barcoded DNA retrieved from different parts of the small intestine. Data are presented as box plots with min-max whiskers. FIG. 15C shows the percentage of each barcoded plasmid in the population retrieved from the intestinal epithelium region in the small intestine. FIG. 15D presents CRE-mediated TdTomato activation (white dots) in Ai14 mice's small intestine and liver at 48 h post administration of C-P₇₃/B₅₇ encapsulating Cre/GFP plasmid. Scale: 200 μm. FIG. 15E shows Levels of PCSK9 and ANGPTL3 in the serum after 17 days from the first administration. (n=5 for controls and n=6 for the group treated with Cas9 plasmid). (f) Changes in total cholesterol, HDL, and triglycerides in the serum of the group treated with C-P73/B57 with the dual-targeting Cas9 plasmid (n=6). Statistical analysis was performed using one-way ANOVA with Tukey's post hoc multiple comparisons for FIG. 15E and paired two-tailed Student's t test for FIG. 15F. Significance presented as *p<0.05, **p<0.01, and n.s., not significant.

FIG. 16 shows representative DLS histograms of C-BX/BY polyplexes at pH 7.4.

FIGS. 17A and 17B show the colloidal stability of the polyplexes. FIG. 17A depicts the size of C-BX/BY polyplexes incubated at room temperature in a PBS buffer (pH 7.4). FIG. 17B depicts the size (left Y-axis) and PDI (right Y-axis) of the polyplexes at pH 1.5.

FIG. 18 is a representative gel electrophoresis of C-BX/BY after incubated with dextran sulfate at A/P ratio of 30 at pH 1.5.

FIG. 19 is a representative gel electrophoresis of C-BX/BY after incubated with bile salt (20 mM) at pH 7.4.

FIG. 20 shows the cell viability of HCT116 cells after 24 h of C-PX/BY transfection. Data are presented as average±SEM. Each dot represents a biological repeat. Significance was determined using one-way ANOVA with Dunnett's post-hoc test (compared with the C-PX/B0 group) and presented as *, p<0.05 (n=3).

FIG. 21 shows representative confocal images of each channel in FIG. 14C (Scale bar: 10 μm).

FIG. 22 is a representative gel electrophoresis of the polyplexes after incubated with dextran sulfate (A/P ratio of 20) in the presence of ATP (3 mM).

FIG. 23 demonstrates transport validation of C-PX/BY polyplexes through the mucus layer. In vitro transport of Cy5-labeled plasmid encapsulated in the C-PX/BY polyplexes through mucus layer on transwell plates were measured. Significance was determined using one-way ANOVA with Dunnett's post-hoc test (compared with the C-PX/B0 groups) and presented as *p<0.05 (n=3).

FIGS. 24A-24D depict dual-targeting Cas9-T2A-EGFP plasmid designed in Example 4. Designs of 20 bp DNA barcoded plasmids are shown in FIG. 24A and of Pcsk9- and Angptl3-targeting plasmids are shown in FIG. 24B. Sanger sequencing was used to confirm the gRNA construction of the dual-targeting plasmid (FIG. 24C). For Pcsk9 gRNA verification, the pBR322ori forward primer (GGGAAACGCCTGGTATCTTT, SEQ ID NO. 1) was used, and for Angptl3 gRNA verification, the CMV-En reverse primer (TGGAAAGTCCCTATTGGCGT, SEQ ID NO. 2) was used. HEK293T transfection was used to confirm Cas9-T2A-EGFP construction (FIG. 24D).

FIG. 25 shows the size (bar) and PDI (circle) of reconstituted C-PX/BY polyplexes after lyophilization. Data are presented as average±SEM (n=3).

FIG. 26 shows the profile of alanine aminotransferase (ALT), aspartate aminotransferase (AST), albumin, and blood urea nitrogen (BUN) before and after the polyplex treatment. Data are presented as average±SEM, n=5 and n=6 for the PBS control and C-P73/B57 polyplex groups, respectively.

FIG. 27 shows the editing performance at the target Pcsk9 and Angptl3 sites. Modified % determined by Sanger sequencing with TIDE analysis. Data are presented as average±SEM, n=5 for the group treated with control gRNAs and n=6 for the group with Pcsk9/Angptl3 targeting gRNAs (abbreviations: CTRL, control gRNAs; P+A, Pcsk9/Angptl3 targeting gRNAs).

FIGS. 28A-28B show LCN2 effects on lean body mass and endurance. FIG. 28A represents the lean body mass (% of total body mass) (assessed by Bruker® Minispec™ nuclear magnetic resonance, Bruker® Optics) of 8-week-old male wild-type (WT) mice treated with vehicle or 150 ng/g/day recombinant LCN2 for the time indicated. n=8/group. Mean±SEM. * P<0.05, Student's t test. FIG. 28B shows exercise distance of 6-month-old Lcn2−/− and WT littermates individually housed in cages equipped with voluntary running wheels (Minimitter, Bend, OR). Data were recorded for 14 days following a 7-day acclimation. (n=4/group). Mean±SEM. *, P<0.05, Student's t test.

FIGS. 29A-29B show changes in energy expenditure in mice overexpressing LCN2 and LCN2 knockout mice. FIG. 29A shows lean and fat body mass (% of total body mass) of 10-week-old TgOsb-LCN2 and WT littermate male mice (assessed by Bruker® Minispec™ nuclear magnetic resonance, Bruker® Optics). WT, n=7; TgOsb-LCN2, n=10. Mean±SEM. *, P<0.05, Student's t test. The percent lean and fat body mass of 12-week-old LCN2Osb−/− and WT littermate male (n=13) or 8-week-old female (WT, n=10; LCN2Osb−/−, n=11) mice (assessed by Bruker® Minispec™ nuclear magnetic resonance, Bruker Optics) is shown FIG. 29B. Mean±SEM. *, P<0.05, Student's t test.

FIG. 30 shows relative expression levels (fold over vehicle) of Myf5, Mrf4, MyoD and MyoG in the muscles indicated of WT male mice treated with the indicated doses of recombinant LCN2 or vehicle for 16 weeks. Mean±SEM. *, P<0.05, Student's t test.

FIG. 31 shows relative expression levels (fold over vehicle) of the indicated genes in tibialis anterior of 12-week-old male or female Lcn2osb−/− and WT littermates (n=8/group). Mean±SEM. *, P<0.05, Student's t test.

FIGS. 32A-32B show relative expression levels (fold over vehicle) of MyHCIIa and MyHCIIb in EDL of 12-week-old male Lcn2osb−/− or WT littermates (FIG. 32A) or 8-week-old WT male mice treated with the indicated doses of recombinant Lcn2 or vehicle (n=6/group) for 16 weeks (FIG. 32B). Mean±SEM. *, P<0.05, Student's t test.

FIG. 33 shows relative expression levels (fold over vehicle) of myoglobin and troponin I in tibialis anterior of 12-week-old female Lcn2osb−/−(n=5) or WT littermates (n=3). Mean±SEM. *, P<0.05, Student's t test.

FIGS. 34A-34B show relative expression levels (fold over vehicle) of the indicated genes in tibialis anterior of 12-week-old male Lcn2osb−/− or WT littermates (FIG. 34A) or 8-week-old WT male mice treated with 150 ng/g/day recombinant Lcn2 (n=7) or vehicle (n=8) for 16 weeks (FIG. 34B). Mean±SEM. *, P<0.05, Student's t test.

FIG. 35 shows relative expression levels (fold over vehicle) of MyoD, Myf-5, Mrf-4 and MyoG in C2C12 cells treated with increasing concentrations of recombinant Lcn2 for 4 h. Mean±SEM. *, P<0.05, Student's t test.

FIG. 36 shows relative expression levels (fold over vehicle) of Nrfl and Pgcla in C2C12 cells treated with increasing concentrations of recombinant LCN2 for 4 h. Mean±SEM. *, P<0.05, Student's t test.

FIG. 37 show relative expression levels (fold over vehicle) of AcylcoA, Mcad and Ucp2 in C2C12 cells treated with increasing concentrations of recombinant Lcn2 for 4 h. Mean±SEM. *, P<0.05, Student's t test.

FIG. 38 shows Western blot analysis of phosphorylated (p-) or total (t-) PKA and CAMP production (assessed by ELISA (Enzo Life Sciences®) in C2C12 cells treated with increasing concentrations of recombinant LCN2. Mean±SEM.

FIG. 39 depicts microscopic images of LCN2+ and LCN-myocytes.

FIG. 40 represents a diagram of how LCN2 regulates lean body mass.

FIG. 41 shows a programmable gene editing system that can gene-edit at least: 3 tissues, or 3 cell types based on known biological mechanisms.

FIGS. 42A-42F show liver gene editing via oral delivery. FIG. 42A shows gel electrophoresis of barcode PCR amplicon extracted from the muscularis externa (outer part; top) and intestinal epithelium layer (inner part; bottom) in the small intestine at 24 h post administration. FIG. 42B represents the amount of barcoded DNA retrieved from different parts of the small intestine. FIG. 42C shows the percentage of each barcoded plasmid in the population retrieved from the intestinal epithelium region in the small intestine. FIG. 42D shows CRE-mediated TdTomato activation (white dots) in Ai14 mice's small intestine and liver at 48 h post administration. FIG. 42E shows levels of PCSK9 and ANGPTL3 in the serum after 17 days from the first administration. Changes in total cholesterol, HDL, and triglycerides in the serum of the group treated with C-P73/B57 are shown in FIG. 42F.

FIGS. 43A-43C. show oral delivery of the dCas9/VPR/EGFP/gRNA-TTN plasmid to activate the TTN gene. FIG. 43A depicts IHC staining images of TTN illustrating TTN expression. Quantification of the IHC images shown in FIG. 43B and TTN mRNA levels in the GI tract represented in FIG. 43C shows elevated TTN expression in various segments of the GI tract.

FIGS. 44A-44D show gene editing in MLN pf Ai14 reporter mice via oral delivery of Cre plasmid. FIG. 44A illustrates representative fluorescence images and FIG. 44B shows immunohistochemical (IHC) staining images. Percentage of tdTomato-positive cells in the MLN are represented in FIG. 44C. FIG. 44D shows the cell types gene edited in the MLN. The YF-NP group exhibits a greater total tdTomato expression in the MLN compared to the NP group lacking the yeast membrane coating. The majority of tdTomato-positive cells are identified as macrophages (F4/80 positive) and dendritic cells (CD11c positive).

FIGS. 45A-45E show flash nanocomplexation (FNC) technology for NP scale-up production and membrane coating. FIG. 45A depicts various FNC devices developed for NP production and coating applications. FIG. 45B illustrates improved size and size distribution of NP produced by FNC compared to NP generated by bulk mixing. FIG. 45C shows improved colloidal stability of NP coating with cell membrane using FNC. FIG. 45D shows decrease of zeta potential to indicate the successful cell membrane coating on NP. FIG. 45E shows lower toxicity of NPs with the yeast membrane coating.

FIG. 46 shows elevated tdTomato expression in the MLN. The majority of tdTomato-positive cells are macrophages (F4/80 positive) and dendritic cells (CD11c positive).

FIG. 47A-47E show gene editing in Peyer's patches following oral administration of YF-CPNP.

FIG. 48 shows an illustration of gene editing in MLNs through oral delivery. The in vivo study was conducted in mice. The aqueous solution nanoparticles was administered via oral gavage and entered the GI tract. At the GI epithelium, the nanoparticles were uptaken by M cells and translocated to the PP below the epithelium. Due to the connectivity between the PPs and the MLNs, the nanoparticles were eventually transported to the MLNs by APCs.

show in vivo gene editing in Ai14 reporter mice following oral administration of YF-CPNP.

FIGS. 49A-49D show the in vivo validation of transportation route of YF-containing nanoparticles (YFNPs) compared with YF-free nanoparticles (NPs). FIG. 49A shows a diagram of the LoxP flanked STOP cassette upstream of tdTomato. When Cre recombination occurs, the STOP cassette gets removed and tdTomato is transcribes. FIG. 49B represents a schematic timeline of oral gavage treatment and sacrifice of Ai 14 mice. FIG. 49C shows representative fluorescence images showing tdTomato expression (red) in the liver and various segments of the GI tract, including the stomach, duodenum, jejunum, ileum, and colon, while the cell nuclei were stained with DAPI (blue). FIG. 49D shows representative fluorescence images showing gene editing in macrophages and M cells in the GI epithelial villi and the PPs.

FIG. 50 shows oral gene editing in Ai14 Mice.

FIGS. 51A-51F in vitro design and optimization of YF-CPNP for gene delivery.

FIG. 52 shows gene editing with YF-CPNP in diverse cell types of mesenteric lymph nodes (MLN).

FIGS. 53A-53C shows Titin (TTN) gene activation after oral administration of YF-CPNP.

FIGS. 54A-54C show in vivo gene editing in Ai14 reporter mice following oral administration of YF-CPNP

FIG. 55 shows an illustration of siRNA delivery using the CS-g-bPEI (PBA) platform. The versatility of the siRNA delivery system stems from a gene carrier whose composition can be precisely tuned for charge density, endosomal escape capability, and efficient release of siRNA.

FIGS. 56A and 56B show the H1 NMR of succinylated-CS, CS-g-bPEI and CS-g-bPEI (PBA).

FIGS. 57A and 57B show the in vitro assessment of siRNA-induced gene silencing. FIG. 57A shows images of GFP expression in U2OS-EGFP cells following treatment with various polyplexes containing siRNA. FIG. 57B shows the percentage of GFP-positive cells following treatment with CP and CPB polyplexes.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

As used herein, the term “encapsulation” refers to the process of enclosing or surrounding therapeutic agents, which include proteins (such as Lipocalin 2, osteocalcin, or a nuclease), nucleic acids (such as siRNA, guide RNA, or a protein-encoding plasmid), or small molecules, with nanoparticles.

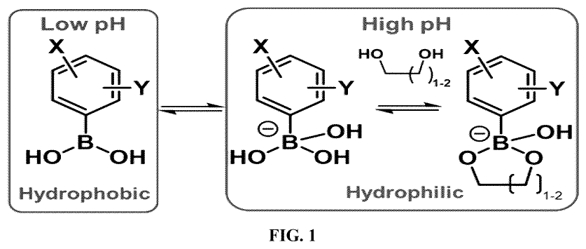

As used herein, the terms “phenyl boric acid” and “phenylboronic acid” are used interchangeably and are abbreviated as “PBA”. These terms refer to unsubstituted PBA as well as derivatives such as compounds with one or more substituents on the phenyl group. Substituents on the phenyl group include halogen atoms (Br, Cl, F) as well as carboxylic acids, aromatic groups (including substituted and unsubstituted phenyls), and alkyl groups (including linear and branched alkyls with or without substituents). One derivative is 2-fluro-4-carboxyphenyl boric acid. As illustrated in FIG. 1, PBA can undergo a pH-dependent equilibrium between hydrophobic and hydrophilic conformations.

As used herein, the term “CRISPR-Cas system” refers to an enzyme system including a guide RNA sequence that contains a nucleotide sequence complementary or substantially complementary to a region of a target polynucleotide, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. CRISPR-Cas systems may contain engineered and/or mutated Cas proteins. CRISPR-Cas systems may contain engineered and/or programmed guide RNA.

As used herein, the term “guide RNA” refers to a RNA containing a sequence that is complementary or substantially complementary to a region of a target DNA sequence. A guide RNA may contain nucleotide sequences other than the region complementary or substantially complementary to a region of a target DNA sequence. A guide RNA may be a crRNA or a derivative thereof, e.g., a crRNA: tracrRNA chimera.

As used herein, the term “nuclease” refers to an enzyme capable of cleaving the phosphodiester bonds between the nucleotide subunits of nucleic acids; the term “endonuclease” refers to an enzyme capable of cleaving the phosphodiester bond within a polynucleotide chain; and the term “nickase” refers to an endonuclease which cleaves only a single strand of a DNA duplex. The term “Cas9 nickase” refers to a nickase derived from a Cas9 protein, typically by inactivating one nuclease domain of Cas9 protein.

As used herein, the term “residue” refers to the remaining portion of a moiety or a reactant after the reactant has reacted to form the polymeric product disclosed herein.

As used herein, the term “polyplex” refers to a complex of a polymer and nucleic acid, such as DNA used in gene therapy.

Disclosed herein an oral formulation that ensures the bioavailability of peptide and protein drugs and gene therapy products when administered orally. To maximize the oral delivery efficacy, it is essential for oral carriers to fulfill multiple functions: (i) safeguard the encapsulated cargos across a wide pH range, spanning from pH 1.5 in the stomach to pH 7.5 in the intestine, (ii) penetrate the gut epithelium, and (iii) facilitate efficient payload release in the targeted organ or tissue. However, achieving these functions simultaneously can be challenging and hampers the practical implementation of nanocarriers.

The disclosed oral formulation comprises (1) a polymer of chitosan (CS) grafted with branched polyethyleneimine (PEI) or of phenyl boric acid (PBA)-functionalized CS grafted with branched PEI and (2) a therapeutic agent, where the therapeutic agent is encapsulated by the polymer. Accordingly, the therapeutic agent may be a peptide drug, protein drug, or gene therapy product where exposure to the gastric and intestinal environments limits these therapeutic agents' efficacy and thus bioavailability.

In this disclosure, we systematically addressed these obstacles of bioavailability of an oral formulation by developing a series of functionalized chitosan (CS) polymers: CS grafted with branched polyethyleneimine (PEI) or phenyl boric acid (PBA)-functionalized CS polymers grafted with branched PEI. Chitosan, a natural polysaccharide derived from chitin found in crustacean shells, is available over the counter in its fibrous form and used in supplements for weight management and cholesterol control. Chitosan serves as a versatile carrier for a wide array of therapeutic payloads, including small molecules, proteins, and nucleic acids, owing to its safety and biocompatibility.

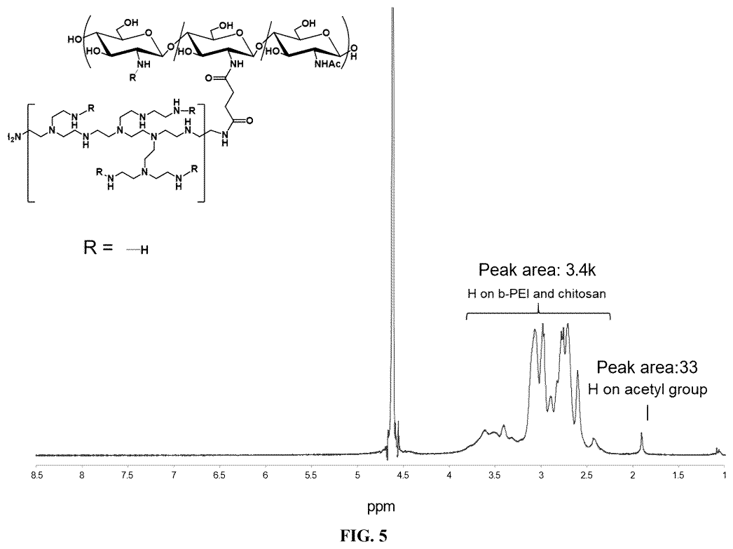

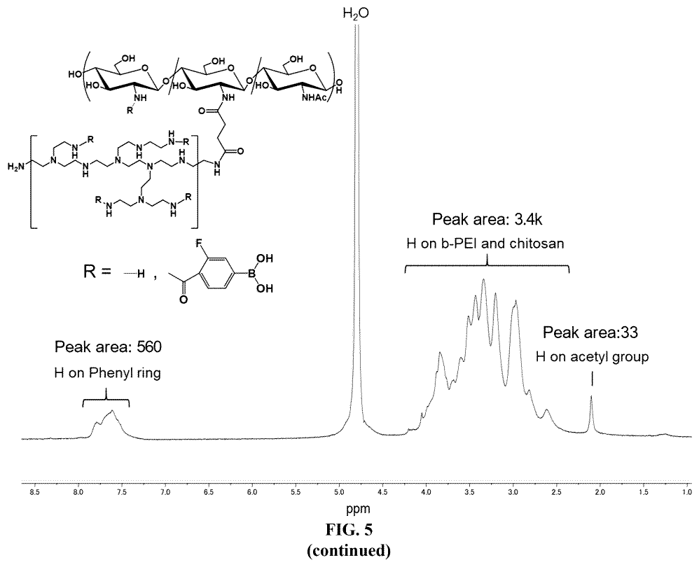

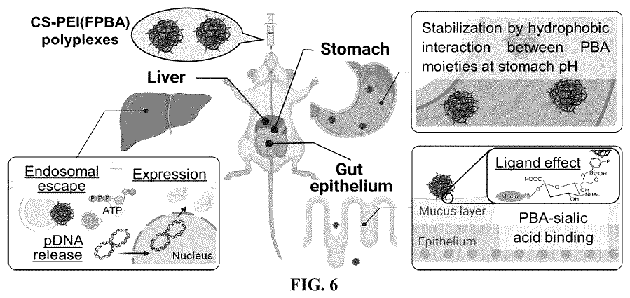

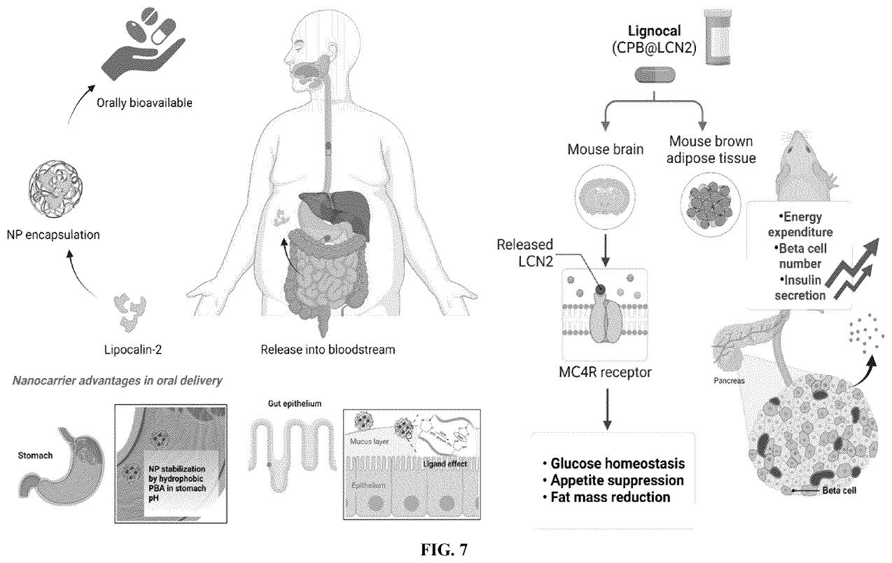

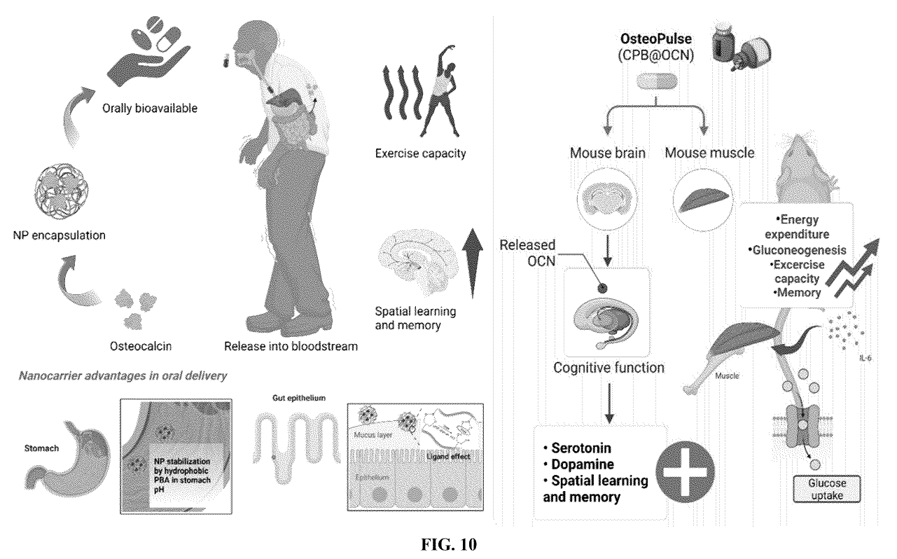

Utilizing PBA moieties in the polymer of the oral formulation provides benefits to address the obstacle to oral bioavailability. Firstly, the hydrophobic interactions among PBA moieties play a crucial role in stabilizing the polymer and safeguarding the encapsulated therapeutic agent, particularly in acidic conditions encountered in the stomach. Secondly, the mucoadhesive properties of the PBA moiety enable it to act as a ligand, facilitating the transport of the nanoparticles through the mucus layer. Thirdly, the PBA functional group can interact with glycoproteins on the cell membrane surface, triggering cellular endocytosis. After entering the target cells, PBA can enhance endosomal escape via hydrophobic interaction at late endosomal pH (pH 5.5) and subsequently binds to cytoplasmic adenosine triphosphate (ATP), which further promotes plasmid unpackaging. PBA exhibits a pH-dependent equilibrium between hydrophobic and hydrophilic conformations and can establish an ester linkage with the diol group when in a hydrophilic state, influenced by the pH and diol concentration (Yoshinaga et al., 2023). See, FIG. 1. This unique dual-responsive behavior enables the polymer to effectively overcome the aforementioned obstacles of oral delivery, as illustrated in FIGS. 7 and 10.

In the selection of PBA polymers, 4-carboxy-3-fluoro PBA (FPBA) is one of the preferred PBA polymers for select oral formulations owing to its distinctive attributes, notably enhanced binding affinity under specific conditions. When deliberating on the choice of PBA for an oral formulation, several fundamental factors necessitate consideration, including the chemical compatibility of the PBA with the therapeutic agent, the PBA's stability within the formulation across varying conditions encompassing pH and temperature fluctuations, and the solubility of the PBA within the formulation matrix.

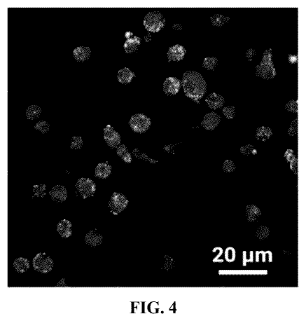

An additional schematic illustration of the in vivo behavior of CS-PEI (FPBA) polyplexes after oral administration is presented in FIG. 6. The instant polymer systems stabilize the subtherapeutic in the acidic conditions of the stomach. Upon entry into the intestine, the polymer system allows improved interaction with the mucus layer and penetrate the gut epithelium. The polymer then also facilitates efficient payload release in the targeted organ or tissue.

In some aspects, the PBA-functionalized chitosan grafted with PEI is a block copolymer. In a particular embodiment, the polymer is of the formula CS-PEI_{X%}(PBA)_{Y%}, where CS is a chitosan residue, PBA is a phenyl boric acid residue, and PEI is a branched polyethyleneimine polymer residue, and where X % is the PEI grafting ratio and Y % is the PBA introduction ratio. The mass grafting percentage is calculated by comparing the mass (or weight) of the grafted component (in this case, PEI) to the total mass of the resulting polymer. It is expressed as a percentage. In some embodiments, the b-PEI grafting ratio for CS-PEI polymer is 35-45%, 37-43%, 39-40%, 39%, 40%, 65-75%, 70-75%, 72-73%, 73%. The introduction ratio is a mass ratio, defined as the weight percent of the reacted PBA component. In certain embodiments, the PBA introduction ratio is 5-20%, 5%, 16%, 18%, 20-30%, 20-25%, 23%, 24%, 26%, 28%, 30%, 49-59%, 49%, 45-59%, 55-59%, 57%, 59%, 65-75%, 65-70%, 68%, or 75%.

The conversion ratio in chitosan-polyethyleneimine (PEI) conjugated with phenyboronic acid refers to the extent to which the primary amino groups on the chitosan-polyethyleneimine conjugate have been modified into phenyboronic acid groups. For example, the 16% conversion ratio for the 39% branched PEI-grafted chitosan indicates that out of all the primary amino groups present in the chitosan-PEI polymer, approximately 16% of them have undergone the reaction for conjugation. The remaining amino groups might still be in their original form. The degree of substitution (39%) for bPEI-grafted CS indicates the extent of the modification. The CS was first reacted with predetermined amounts of succinic anhydride to yield succinated CS with a distinct DS of succinyl groups at the N-positions on its backbone. The PEI was then conjugated to the carboxyl group of the as-prepared succinated CS in the presence of excess 1-ethyl-3-(3-dimethylaminopropyl) carbodiimide (EDC)/N-hydroxysuccinimide (NHS), ensuring that almost all the succinyl groups were coupled with PEI. The grafting ratio for PEI means the degree of substitution in this step.

In some embodiments, the branched PEI has a molecular weight of about 600 Da to about 10000 Da, for example, 800 Da or 2000 Da. In some embodiments, the PBA is 4-Carboxy-3-fluorophenylboronic acid. In some embodiments, the chitosan has a molecular weight of about 1000 Da to 40000 Da, for example, 2000 Da.

The amount of therapeutic agent is 1.5-22%, 1.8-21%, 1.5-5%, 2-5%, 3-10%, 2-5%, 5-20%, 10-20%, or 15-20% by weight relative to the amount of polymer. In some embodiments where the therapeutic agent comprises a protein, the amount of the therapeutic agent in the oral formulation is 1.5-22%, 1.8-21%, 1.5-5%, 2-5%, 3-10%, 2-5%, 5-20%, 10-20%, or 15-20% by weight relative to the amount of polymer.

In some aspects, the loading capacity of the polymer is 2-3%, 3-4%, 4-5%, 15-16%, 16-17%, 17-18%, 18-19%, or about 17.8% by weight. In certain embodiments, the amount of LNC2 loaded into the polymer is 1.75 to 2.2 weight % or 17.6 to 22 weight % relative to the total weight of the LNC2 and CS-PEI_{X%}(PBA)_{Y%} polymer. Likewise, the loading of a CRISPR/Cas system is loaded into the polymer is 4.3 weight % or 4.6 weight % relative to the total weight of the CRISPER/Cas system and CS-PEI_{X%}(PBA)_{Y%} polymer.

As demonstrated in the examples, the oral formulation is suited for oral delivery of therapeutic agents such as lipocalin 2 (LCN2), osteocalcin (OCN), plasmid DNA for providing gene therapy, siRNA, and a CRISPR/Cas system. Through careful modulation of PBA decoration, b-PEI grafting ratios, and succinylation percentages, an optimal formulation for the oral delivery of LCN2, OCN, a CRISPR/Cas system, plasmid, and siRNA have been obtained. The ionic interactions between the anionic carboxylate groups on the therapeutic agent (such as LCN2) and the cationic species within the polymer system play a pivotal role in achieving this affinity. The other tested therapeutic agents have similar affinities. Consequently, we were able to systematically screen and engineer the polymer with enhanced binding affinity for LCN2, OCN, a CRISPR/Cas system, siRNA, or plasmid, further improving its effectiveness as an oral delivery carrier.

As shown in the Examples, the cytotoxicity of the synthesized CPB polymers was investigated in a model system with hematopoietic stem progenitor cells. The IC₅₀ values of the polymers were found to range from 50 to 400 μg/mL, depending on the specific introduction ratios employed. We observed a correlation between cytotoxicity and the grafting ratios of b-PEI and PBA. Specifically, lower b-PEI conjugating ratios were found to decrease the overall charge density of the polymer, resulting in higher IC₅₀ values. Similarly, higher PBA grafting ratios contributed to reduced overall charge density, which also influenced the cytotoxicity, albeit to a lesser extent.

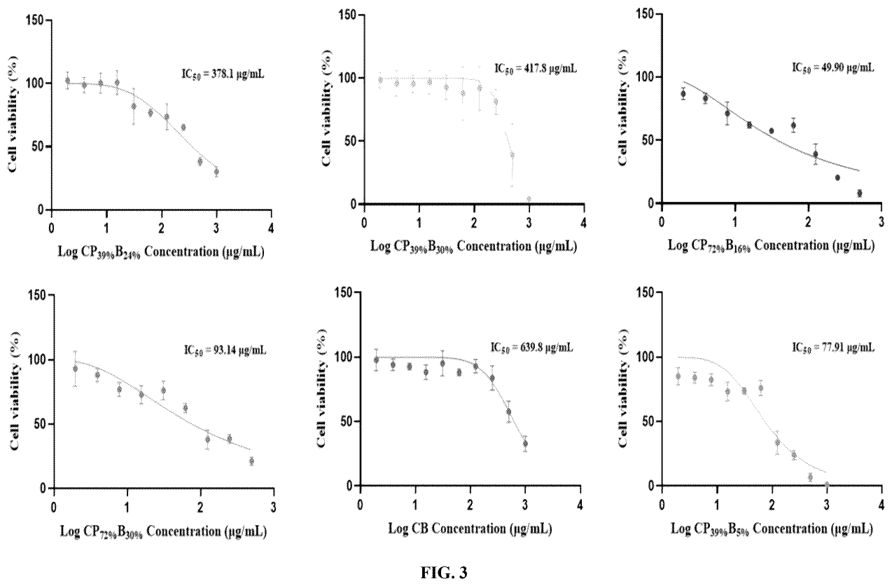

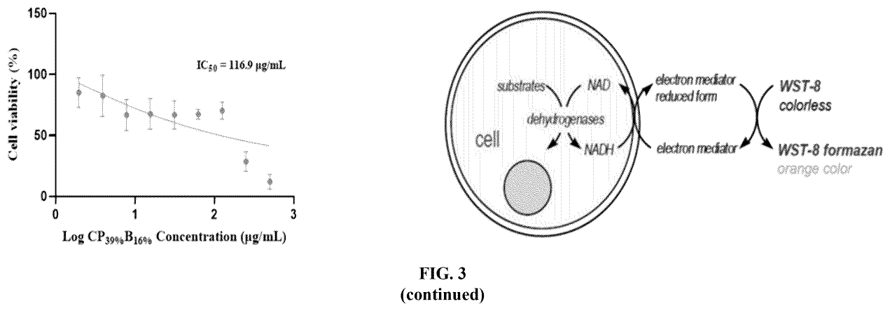

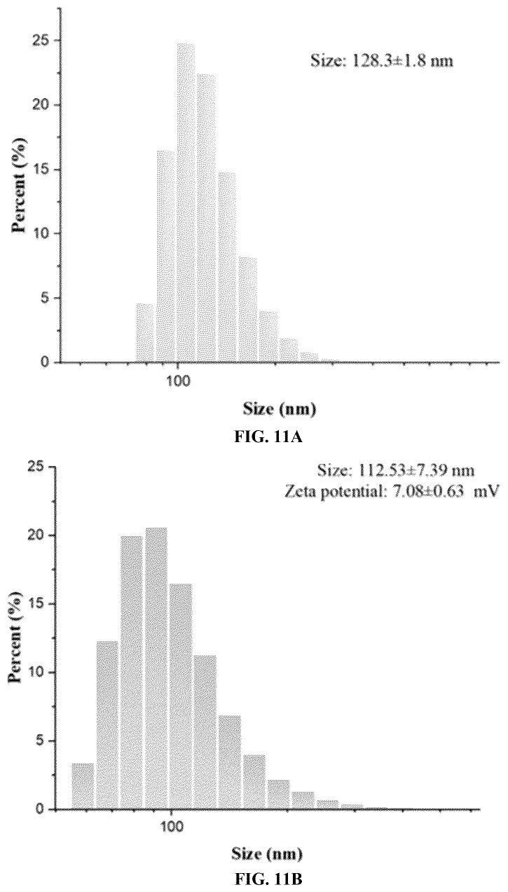

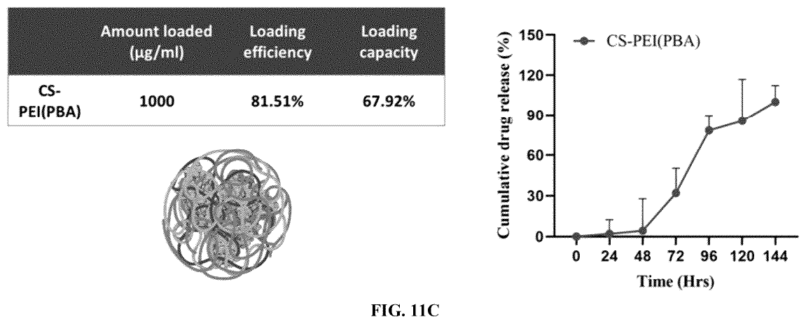

FIG. 3 presents cytotoxicity screening of the established CS-PEI (PBA) library against hematopoietic stem progenitor cells results. The intracellular distribution of the nanoparticles was observed using confocal laser scanning microscopy. Confocal laser scanning microscopy (CLSM) is a technique known by those skilled in the art for obtaining high-resolution optical images having depth selectivity and allowing for protein localization in specific cellular compartments. BSA as a model protein was utilized in the study, and the confocal images revealed the successful internalization and endosomal escape of Cy5-labeled protein encapsulated in the CS-PEI39% (PBA) 24% (FIG. 4). We assessed the intracellular distribution of nanoparticles using CLSM. Bovine serum albumin (BSA) served as a model protein, and the confocal images provided unequivocal evidence of the successful internalization and endosomal escape of Cy5-labeled protein encapsulated within the chitosan-polyethyleneimine (CS-PEI) nanoparticles with a specific composition of 39% b-PEI grafting and 24% PBA functionalization (FIG. 6A). In FIG. 11C the polymeric nanoparticle loaded with BSA protein and its corresponding drug release profile is represented.

The oral formulations disclosed herein can also be used to targeting particular cell types for delivery of the therapeutic agent. Cell membrane coating has emerged as a promising approach to improve delivery to specific cells and tissues. In some embodiments, the oral formulation, in particular for gene therapy products, further comprises membrane fragments from a eukaryotic cell. The incorporation of the eukaryotic cell membrane fragment enables delivery of the therapeutic agent to specific cell types with oral administration of the formulation. Rapid mixing of the eukaryotic cell membrane fragment and a polyplex, such as using flash nanocomplexation, enables coating the polyplex with the eukaryotic cell membrane fragment. In other embodiments, the oral formulations can be targeted to specific cells by conjugating targeting ligands to the surface of the encapsulated therapeutic agent. In still other embodiments, the oral formulations can be targeted to specific cells by the incorporation of a targeting polymer, where the targeting polymer coats the chitosan polymers described herein.

In some embodiments, the eukaryotic cell membrane fragment is from yeast (such as B-glucans isolated from a yeast like Saccharomyces cerevisiae). In such embodiments, the therapeutic agent can be targeted for delivery to mesenteric lymph nodes upon oral administration. Other embodiments that target the therapeutic agent in the oral formulation to the mesenteric lymph node is coating the surface of the encapsulated therapeutic agent with a ligand to Dectin-1 receptors, such as beta-glucans.

In some embodiments, the eukaryotic cell membrane fragment is from a liver cell. In such embodiments, the therapeutic agent can be targeted for delivery to the liver upon oral administration. In some aspects, the eukaryotic cell membrane fragment is from hepatocytes. In some aspects, the eukaryotic cell membrane fragment is from HepG2 cells. Other embodiments that target the therapeutic agent in the oral formulation to the liver is coating the surface of the encapsulated therapeutic agent with N-acetylgalactosamine, for example triantennary N-acetylgalactosamine.

In some embodiments, the eukaryotic cell membrane fragment is from an intestinal cell. In such embodiments, gastrointestinal localization of the gene therapy product upon oral administration is enhanced, such as targeting to the colon. In some aspects, the eukaryotic cell membrane fragment is from intestinal epithelial cells, whether from the small intestine or the colon. In some aspects, the eukaryotic cell membrane fragment is from Caco2 cells. Other embodiments that target the therapeutic agent in the oral formulation to specific parts of the gastrointestinal tract is coating the encapsulated therapeutic agent with a targeting polymer. For example, Eudragit S100 and FS30D are polymers that target delivery to the colon.

a. Oral Delivery of Peptide or Protein Drugs

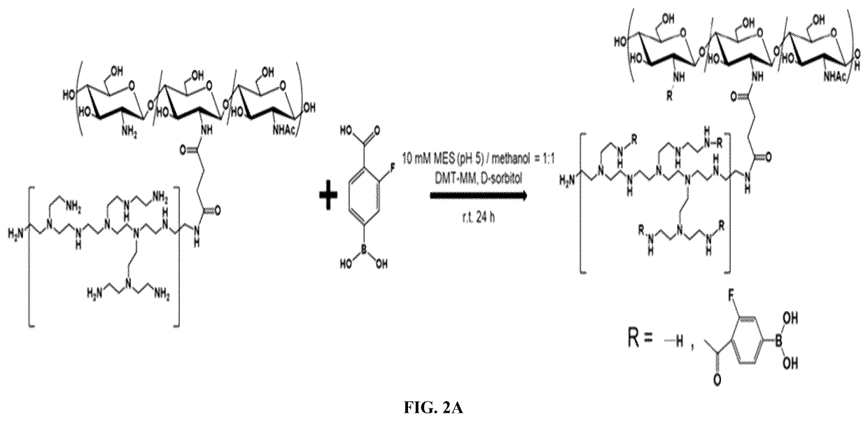

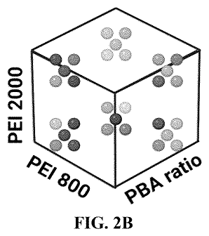

For protein delivery, we synthesized CS-PEI polymers with molecular weights of 15 kDa for CS and 800 Da/2000 Da for b-PEI. “800 Da/2000 Da” signifies two types of branched polyethyleneimine (b-PEI) with molecular weights of 800 Da or 2000 Da. For example, “800 Da” refers to the molecular weight of a specific b-PEI version deployed in chemical synthesis. These polymers were prepared at b-PEI grafting ratios of 39% and 72%. Subsequently, PBA moieties were introduced at various ratios through amide coupling, as depicted in FIG. 2. The conversion ratio of primary amino groups into PBA was determined by both 1H-NMR spectrum and fluorescamine analysis. For the 39% b-PEI-grafted CS, the conversion ratios were found to be 5%, 16%, 24%, 30%, and 49%. Similarly, for the 72% b-PEI-grafted CS, the corresponding ratios were 5%, 16%, 24%, 30%, and 59%. Consequently, a library of 15 PBA-functionalized CS-PEI polymers was prepared, denoted as CS-PEI_{X%}(PBA)_{Y%}, where X and Y represent the b-PEI grafting and PBA introduction ratios, respectively.

FIG. 2 presents the design information for CS-PEI (PBA) nanoparticles for oral therapeutic protein delivery. FIG. 2A shows a synthetic scheme for CS-PEI (PBA). FIG. 2B presents the library design by altering the PEI and PBA ratios.

### Lipocalin 2

In general, this nanoplatform can be deployed to deliver lipocalin 2 (LCN2) to the fat tissues, hypothalamus, and pancreas via oral route. There is currently no available oral formulation for LCN2. Such a formulation is important as oral formulation is the most patient-compliant medication.

Lipocalin 2 (LCN2) is a known bone-derived hormone that can inhibit food intake, increase satiety, increase energy expenditure, and increase insulin secretion. LCN2 is also known as oncogene 24p3 or neutrophil gelatinase-associated lipocalin (NGAL). FIG. 7 illustrates LCN2 targeting the MC4R (Melanocortin 4 receptor) in the brain. MC4R is one of the most potent obesity regulating pathways to induce appetite suppression and increase energy expenditure (Mosialou et al., 2017). Exogenous LCN2 can improve metabolic parameters in obesity by increasing beta islet cells and increasing insulin secretion (Mosialou et al., 2020). Further work has demonstrated that these findings extend to non-human primates (Petropoulou et al., 2020). LCN2 can “brown” white adipose tissue to create a more metabolically active beige adipose tissue. Beige adipose tissue can further increase energy expenditure and improve glucose and lipid clearance resulting in improvements in obesity and glucose homeostasis. In this disclosure, we utilize the weight-controlling properties of LCN2 to develop an orally bioavailable drug termed Lignocal™. Considering the challenges posed by the acidic gastric environment and limited permeability across the intestinal epithelium, the oral delivery of macromolecules, especially proteins, and peptides, has faced significant obstacles. However, we have developed a viable and safe nanosystem for oral administration holds tremendous promise in addressing the requirements for systemic delivery of LCN2. Accordingly, a method of reducing weight in a subject (for example and obese subject) is disclosed. The method comprises orally administering to the subject a formulation of LCN2 encapsulated by a polymer of PBA-functionalized chitosan grafted with branched PEI.

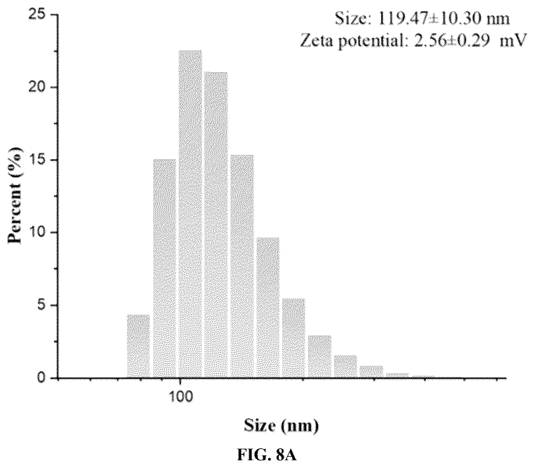

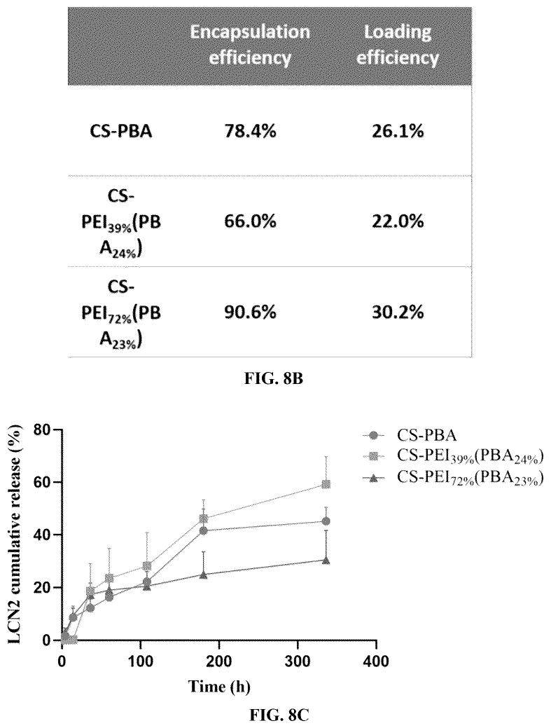

FIG. 8A shows the size and zeta potential measurements, obtained by dynamic light scattering (DLS), of CPB@LCN2 nanoparticles. These nanoparticles are formed by complexing LCN2 protein with tannic acid and then encapsulating them with CS-PEI (PBA) polymers. The average diameter of the nanosystem is 119.47±10.30 nm (FIG. 8A). Notably, at pH 7.4, functionalization with PBA reduced the zeta potential of CPB nanoparticles from 35 mV to approximately 18 mV.

To assess the kinetics of protein release, CPB@LCN2 nanoparticles were subjected to incubation under buffer conditions. The findings revealed a sustained release pattern of LCN2 over a two-week period (FIGS. 8B-8C). Remarkably, the release profile of the nanoparticles could be modulated by altering the grafting ratio, thereby offering the potential for tailored and purposeful administration strategies. These results show that CS-PEI (PBA) block copolymer is a promising platform with effective encapsulation and unpackaging profiles for protein delivery.

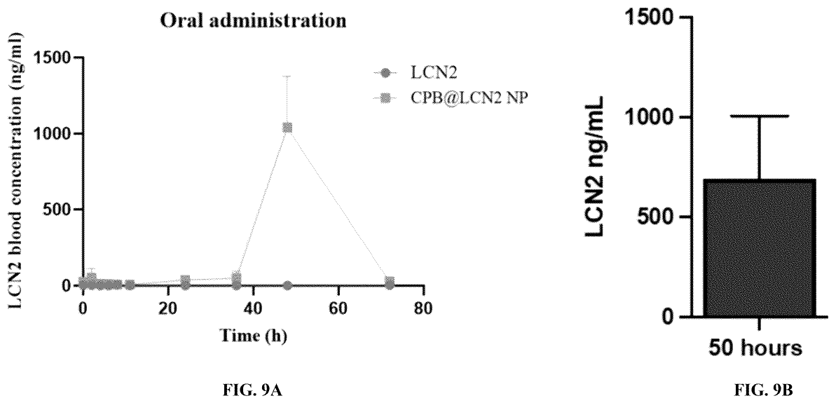

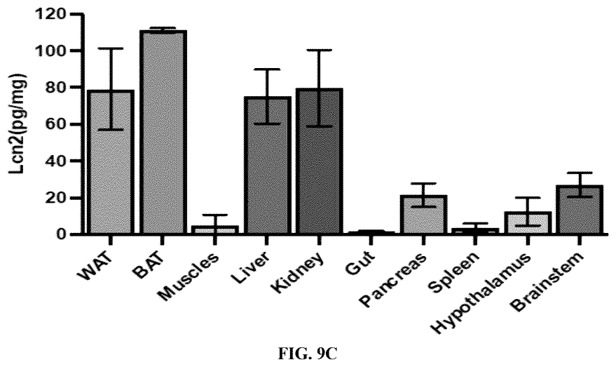

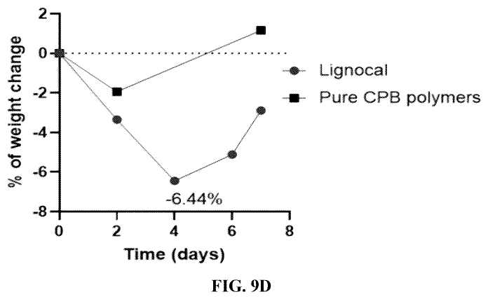

To evaluate the pharmacokinetic characteristics after the oral administration of CPB@LCN2, an Enzyme-linked immunosorbent assay (ELISA) assay was applied to detect LCN2 in the blood serum of the lipocalin 2 knockout mice (LCN2−/−). The naked protein solutions were given orally as comparisons (FIG. 9A). At the 50-hour time point following oral gavage, the blood serum concentration of LCN2 was measured to be approximately 600 ng/ml (FIG. 9B). Subsequently, the knockout mice were sacrificed, and major organs were collected. Tissue lysates were obtained, and a BCA protein assay was conducted to determine the total protein content in each organ, which served as a reference for the subsequent ELISA assay to quantify the LCN2 levels. The results indicate that LCN2 accumulated notably in white adipose and brown adipose tissues, where LCN2 can act to increase energy expenditure. Additionally, it was observed that approximately 16 μg/mg of LCN2 in the hypothalamus, implicating the successful oral delivery of LCN2 in vivo to target the MC4R signaling pathway. Furthermore, it was observed that notable changes in the body weight of LCN2 knockout mice following just one oral gavage, resulting in an approximate 6.44% decrease over a 4 day period when compared to the control group.

FIGS. 4 and 9A-9D show CLSM imaging and an in vivo animal study using the LCN2−/− mice. FIG. 4 shows a CLSM image of Cy5-labeled BSA-loaded nanoparticles. In the figure, blue is cell nuclei, green is late endosomes/lysosomes, and magenta is Cy5-BSA. FIG. 9A shows blood serum concentration of LCN2 in the LCN2−/− mice. FIG. 9B shows blood serum concentration of the knockout mice at the time of organ collection. FIG. 9C presents in vivo biodistribution of LCN2 in different organs at 50 hours. FIG. 9D shows comparison of body weight changes in LCN2−/− mice after oral gavage compared to the pure polymer oral gavage group.

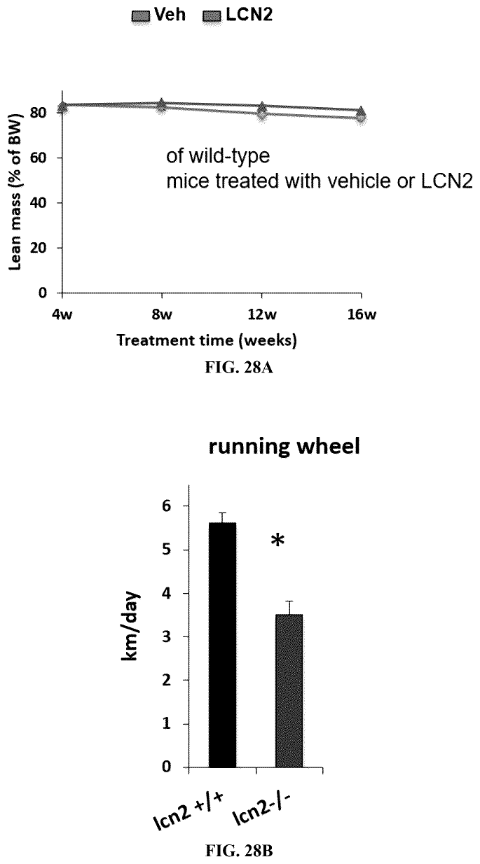

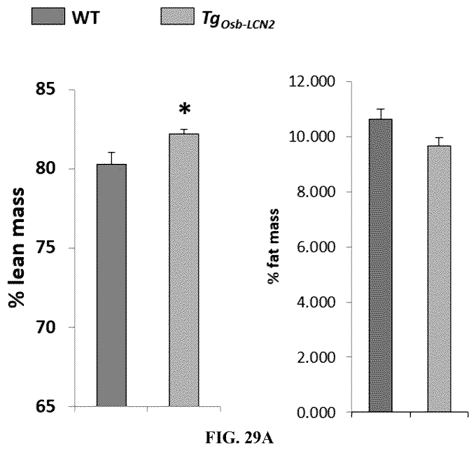

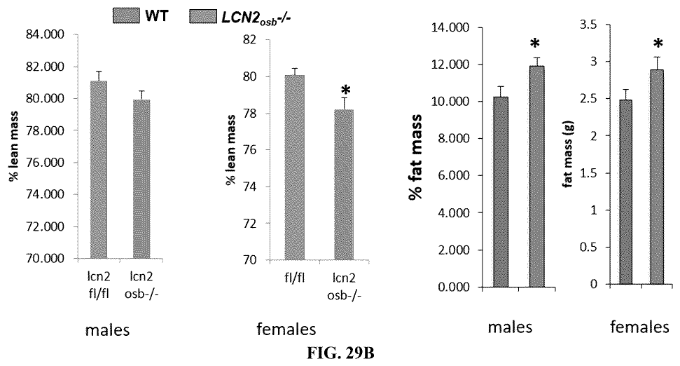

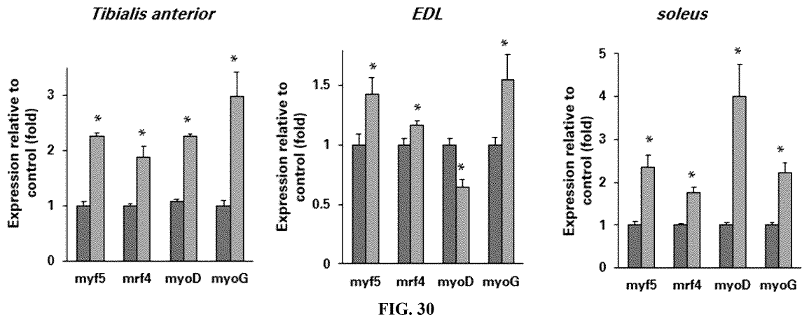

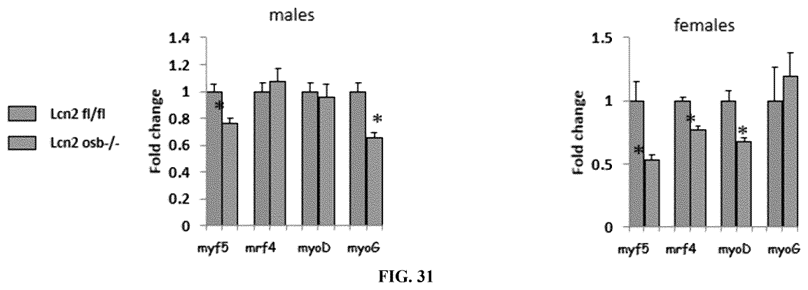

Regarding muscle function, LCN2 is required to maintain lean mass and endurance. FIG. 28A shows lean body mass of 8-week old male WT mice treated with LCN2 compared to those treated with vehicle. Endurance measured as exercise distance of 6-month old LCN2−/− and WT littermates individually housed in cages equipped with voluntary running wheels and recorded for 14 days following a 7-day acclimation is shown in FIG. 28B. The increase in energy expenditure of overexpressed LCN2 can be explained in part from the increase in lean body mass, which is correlated to basal metabolic rate. The knockout mice have a trend towards decreased lean body mass while the transgenic mouse has significantly increased % lean body mass (FIGS. 29A-29B). FIG. 30 shows that LCN2 increases the expression of key myogenic factors. Myogenic regulatory factors, namely myf5, mrf4, myoD and myoG. are transcription factors that regulate myogenesis. In the knockout mice, these myogenic factors either decreased or had little change though seems to trend to decrease. However in male the WT treated with LCN2 there is at least a twofold increase in all myogenic regulatory factors (FIG. 31). This shows directly that LCN2 can affect myogenesis-even in adulthood, which are important in response to recovery from injury.

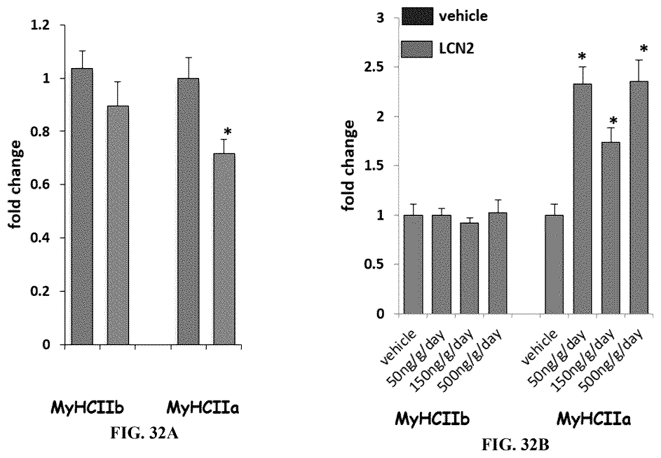

Furthermore, the effect was studied in the specific type of type two muscle fiber. Type IIb is the classical fast twitch, white meat, glycolytic fiber type, whereas type IIa is still considered fast twitch but with oxidative capacity. It is considered in between an in between muscle fiber type between type 1, which are the slow twitch, red meat, oxidative muscle fiber, and classic type IIB. LCN2 increases the amount of myosin heavy chain type IIa compared to type IIb. Both type II fibers are fast twitch, primarily glycolytic muscle fibers, but the type A ones are characterized by more mitochondria and seem to be more oxidative. FIGS. 32A-32B show the relative expression levels of (fold over vehicle) of MyHCIIa and MyHCIIb in EDL of 12-week-old male Lcn2osb−/− or WT littermates (FIG. 32A) or 8-week-old WT male mice treated with the indicated doses of recombinant LCN2 or vehicle (n=6/group) for 16 weeks. This holds true in the H&E stain whereby the tissue looks like a deeper red-indicating more myoglobin and oxidative capacity and more cells with these tiny dots, indicating more mitochondria.

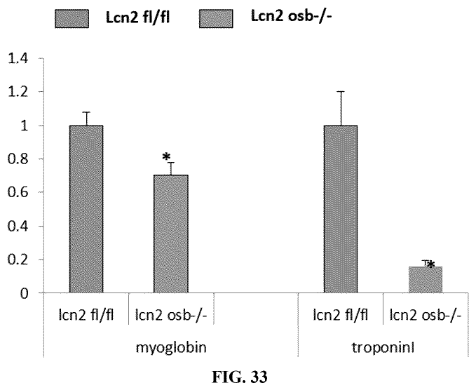

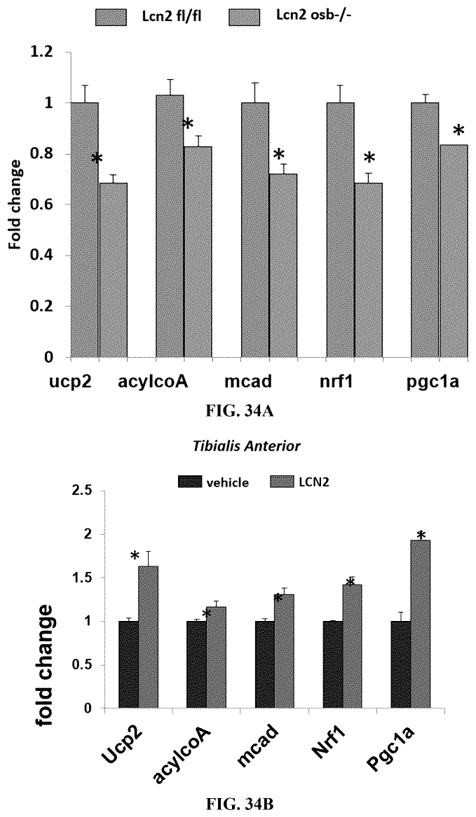

It was also shown that the relative expression of myoglobin and troponin is decreased in LCN2osb−/− mice (FIG. 33). Because it seems like LCN2 is increasing the oxidative capacity in muscle, we studied specific mitochondrial function genes. UCP2 is an uncoupling protein which plays a role in sequestering ROS. AcylCoA dehydrogenase is the and medium chain Acyl CoA dehydrogenase (MCAD) are genes which play a role in beta oxidation of fatty acids and finally nrfl and pgcla work in concert to increase mitochondrial biogenesis. LCN2osb−/− mice trend to a decrease in all mitochondrial associated genes (FIG. 34A). WT mice treated with LCN2 on the other hand have increase in these mitochondrial genes (FIG. 34B).

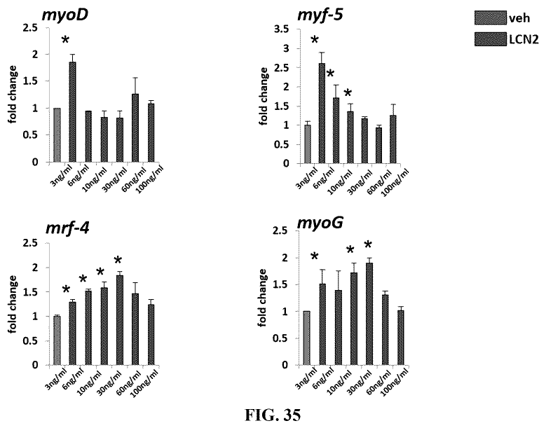

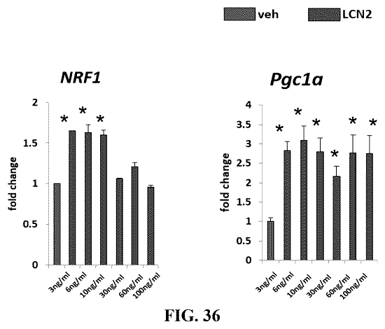

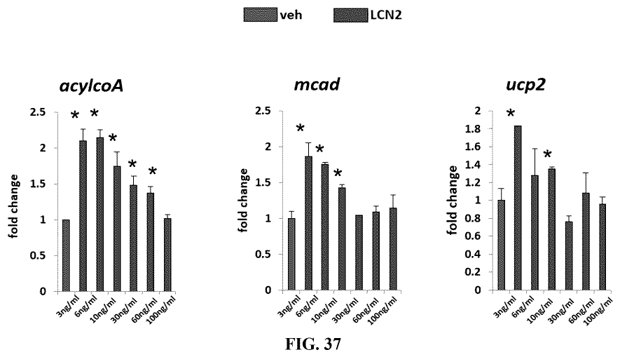

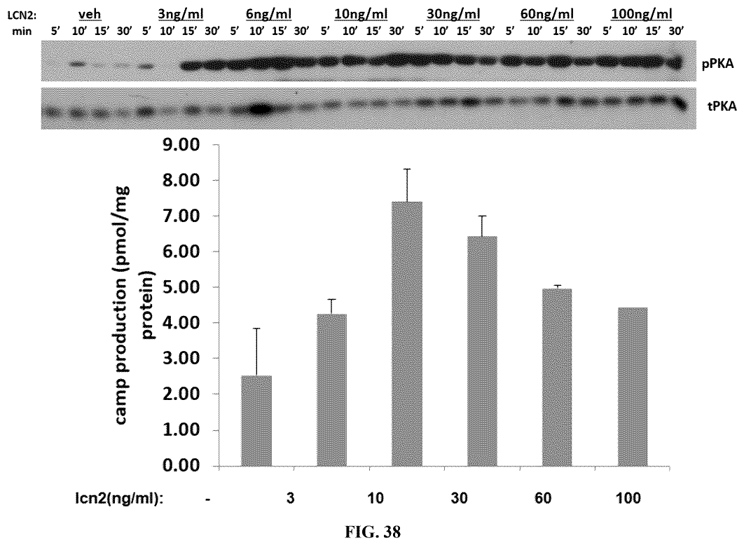

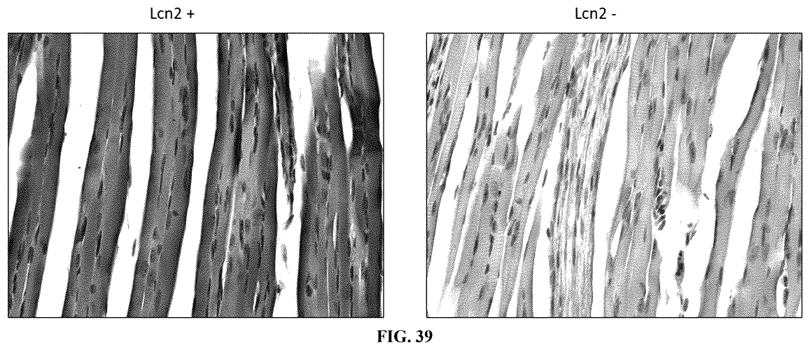

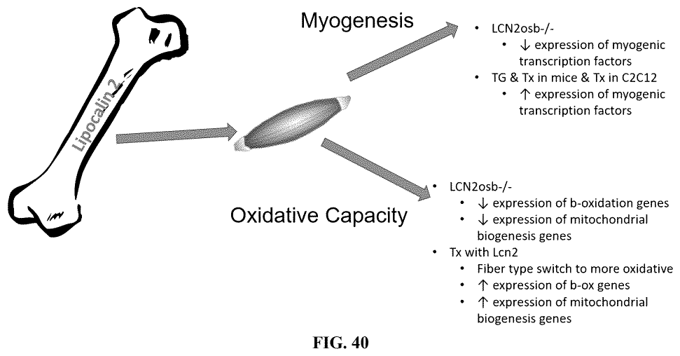

The in vivo results were confirmed in vitro. LCN2 increases the expression of genes that promote myogenesis in C2C12 cells (FIG. 35). The results show the mouse phenotype is expressed in a cell line, and it can be used when cells are needed in culture for a long time. In the in vitro results, it was shown that LCN2 increases the expression of genes that promote mitochondrial biogenesis, such as NRF1 and Pgcla (FIG. 36). The increase in the expression of genes associated with B-oxidation after LCN2 treatment in C2C12 was confirmed (FIG. 37). Western blot and ELISA analysis show that LCN2 induces PKA/cAMP activity in C2C12 cells. FIG. 38A shows the Western Blot analysis of the phosphorylated (p-) and total (t-) PKA production and FIG. 38B represents the CAMP production assessed by ELISA. FIG. 39 shows LCN2+ and LCN2-myocytes. FIG. 40 summarizes how LCN2 regulate lean body mass with an increase in myogenesis and energy expenditure.

### Osteocalcin

The platform can be deployed to deliver an orally bioavailable form of osteocalcin (OCN), which enables the use of the protein for therapeutic benefits, such as enhancing exercise performance. Previously, Karsenty identified the potential of OCN, a bone-derived metabolic regulatory hormone, to reverse age-related sarcopenia and frailty. OCN has a critical role in myoblasts during exercise, where it significantly contributes to enhancing exercise capacity. Osteocalcin acts as a stimulator for interleukin-6 (IL-6) expression in these cells, leading to its subsequent release into the systemic circulation. However, OCN is sensitive to the acidic environment of the stomach and has limited permeability across the intestinal epithelium. The disclosed oral formulation overcomes these challenges.

To maximize the oral delivery efficacy of OCN, it is essential for oral carriers to fulfill multiple functions: (i) safeguard the encapsulated cargos across a wide pH range, spanning from pH 1.5 in the stomach to pH 7.5 in the intestine, (ii) penetrate the gut epithelium, and (iii) facilitate efficient payload release in the targeted organ or tissue. However, achieving these functions simultaneously can be challenging and hampers the practical implementation of nanocarriers.

A series of phenylboronic acid (PBA)-functionalized chitosan polymers grafted with branched polyethyleneimine (CS-PEI), denoted as CPB, was developed for identifying the oral formulation to develop an orally bioavailable OCN drug. PBA exhibits a pH-dependent equilibrium between hydrophobic and hydrophilic conformations and can establish an ester linkage with the diol group when in a hydrophilic state, influenced by the pH and diol concentration. This unique dual-responsive behavior enables the protein carrier to effectively overcome the aforementioned obstacles, as illustrated in FIG. 10. In our study, we explored the potential benefits of utilizing PBA moieties in the design of our polymer system. Furthermore, through careful modulation of PBA decoration and b-PEI grafting ratios, we successfully attained an optimal formulation for the oral delivery of OCN. The ionic interactions between the anionic carboxylate groups on OCN and the cationic species within the polymer system played a pivotal role in achieving this affinity. Consequently, we were able to systematically screen and engineer the polymer with enhanced binding affinity for OCN, further improving its effectiveness as an oral delivery carrier.

Dynamic light scattering (DLS) measurements revealed an average nanoparticle diameter of 112.53±7.39 nm (FIG. 11A). Notably, the PBA functionalization led to a reduction in the ζ-potential of CPB nanoparticles, decreasing from 35 mV to approximately 18 mV at pH 7.4. FIG. 11B presents the zeta potential of CS-PEI (PBA) nanoparticles co-loaded with BSA and OCN. To evaluate the kinetics of protein release, bovine serum albumin (BSA)-loaded nanoparticles were subjected to incubation under controlled buffer conditions. Our findings unveiled a sustained release pattern of BSA over the course of one week (FIG. 11B). Remarkably, the release profile of these nanoparticles could be fine-tuned by adjusting the grafting ratio, offering the potential for tailored and controlled administration strategies. These results emphasize the promise of CS-PEI (PBA) block copolymers as an effective platform with precise encapsulation and release profiles for protein delivery (FIG. 11C).

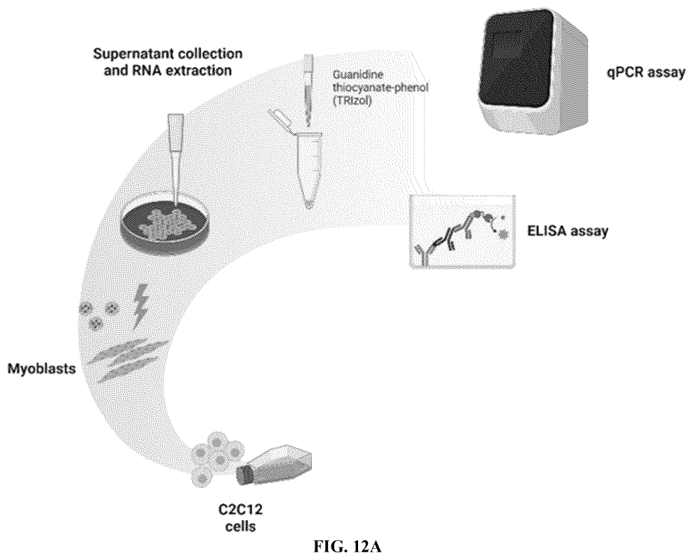

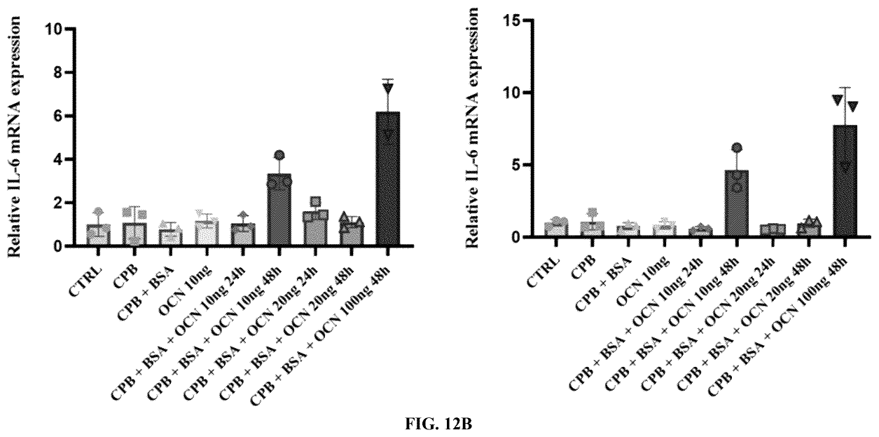

In order to comprehensively assess the performance of the nanocarriers in an in vitro setting, we utilized C2C12 cells, an immortalized mouse skeletal myoblast cell line, known for its heightened sensitivity to osteocalcin levels (FIG. 12A). Given the potential receptor blockade resulting from an excessive protein concentration, we employed a relatively low protein concentration and subjected the cells to treatment with polymeric nanoparticles co-loaded with BSA and osteocalcin (with a mass ratio of co-loaded protein at 10:1).

This ensured the controlled release of the encapsulated osteocalcin at a relatively low dose. After 24 and 48 hours of treatment, cells were collected using Trizol® for RNA extraction. Subsequently, we conducted quantitative polymerase chain reaction (qPCR) experiments to evaluate the in vitro effectiveness of the nanoparticles. Our findings revealed a substantial upregulation of IL-6 expression after a 48-hour treatment, providing strong evidence of the potency of osteocalcin-loaded nanoparticles (FIG. 12B). Thus, also disclosed herein are

b. Oral Delivery of Gene Therapy Products

Currently gene therapy products cannot be administered orally, as the therapeutic genetic material or gene-editing tools and the necessary carrier for functionally delivering the genetic material or gene-editing tools to the patient's cells are sensitive to the low pH environment of the stomach. However, we were able to develop an oral formulation that enhances the stability and intracellular trafficking of vector delivering the therapeutic genetic material or gene-editing tools.

We studied oral administration of insulin-encoding plasmids encapsulated by polyethyleneimine-grafted chitosan (CS-g-bPEI) and encountered promising outcomes. These nanoparticles exhibiting significant accumulation in the liver and successful insulin expression in both the intestine and liver. A single oral dose these CS-g-bPEI nanoparticles provided hyperglycemic protection for more than 10 days in a diabetic mouse model. However, PBA-functionalized chitosan offers improved efficacy of gene therapy products.

For oral formulations with nucleic acids as a therapeutic agent, FPBA is a preferred PBA moieties. As shown in the examples, FPBA functionalization improved the potency of polyplexes (encapsulated gene therapy product) via enhanced stability and better intracellular plasmid trafficking. It also facilitated transport through the mucus because of its interaction with mucin. The optimized polyplex formulation demonstrated the feasibility of systemic gene editing through oral administration for the first time. Notably, the use of the disclosed oral formulation to administer gene therapy avoids the use of viral carriers to deliver functional genetic material or gene-editing tools. Accordingly, disclosed herein is a method of noninvasively administering gene therapy.

In some embodiments of the oral formulation, the residual molar ratio of all amino groups on the polymer to the phosphate groups on the plasmid (N/P ratio) is 60. Accordingly, the optimal grafting ratios of PEI and/or PBA in an appropriate polymer for encapsulating a plasmid or therapeutic oligonucleotide is based on the amount of phosphate groups on the plasmid of therapeutic oligonucleotide.

The screening pipeline and design principle established to validate the oral formulation is also useful for further optimization of other nonviral carriers, including organic and inorganic nanomaterials.

### Plasmid

FPBA was selected to provide efficient endosomal escape ability and ATP-responsive plasmid release in the cytosol because of its acid dissociation constant (pKa) of 7.2, allowing the interaction with intra-cellular ATP at cytosolic pH and the formation of hydrophobic structure at late endosomal pH to trigger destabilization of the endosome. The conversion ratio of primary amino groups into FPBA at 16%, 26%, 37%, 51%, and 68% for the 40% b-PEI-grafted CS, and 18%, 28%, 41%, 57%, and 75% for the 73% b-PEI-grafted one (FIG. 5). The moderately stabilized polyplexes, C-P₄₀/B₂₆ and C-P₇₃/B₂₈, showed the best in vitro transfection efficiency among all the polymer candidates, even in the presence of a mucosal layer.

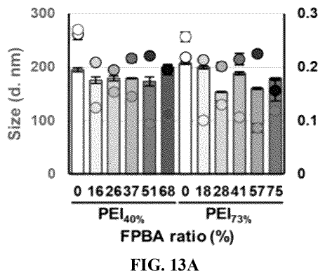

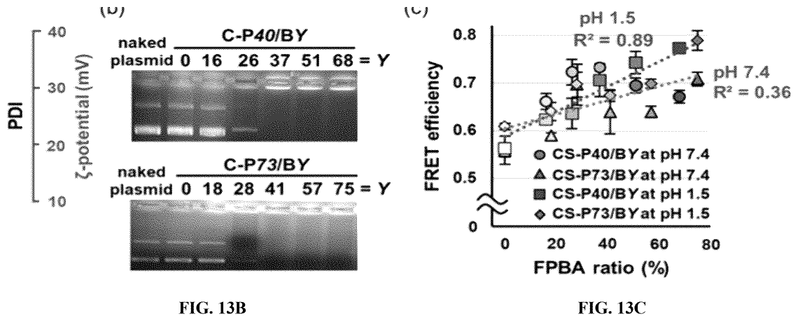

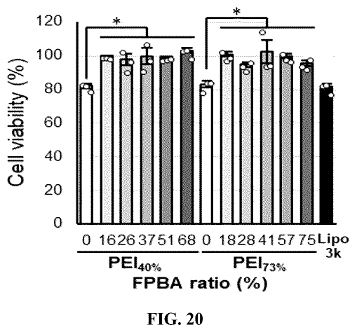

When considering the cytotoxicity, C-PX/B0 is similar to Lipofectamine™ 3000, but FPBA modification reduced the cytotoxicity (FIG. 20). This may be due to negatively charged FPBA moieties decreasing charge density, as shown in the ζ-potential measurements (FIG. 13A).

### Gene-Editing Tools

Effective delivery of CRISPR-Cas9/dCas9 elements is crucial to maximize therapeutic potential. Despite extensive efforts globally to improve delivery of gene editing elements for somatic genome editing, achieving gene editing via oral delivery has remained an elusive challenge.

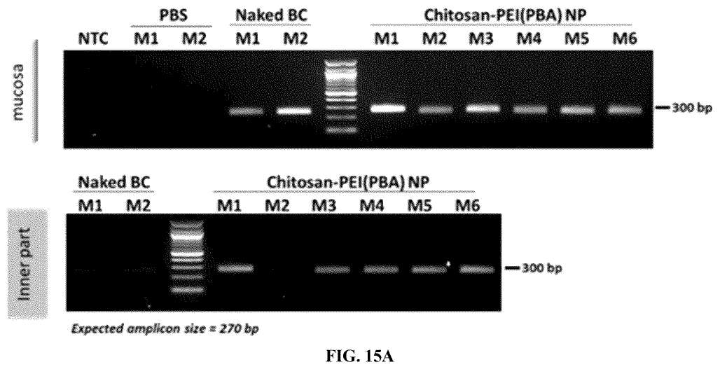

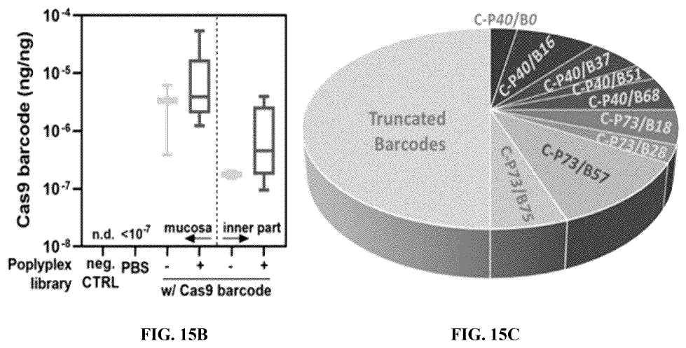

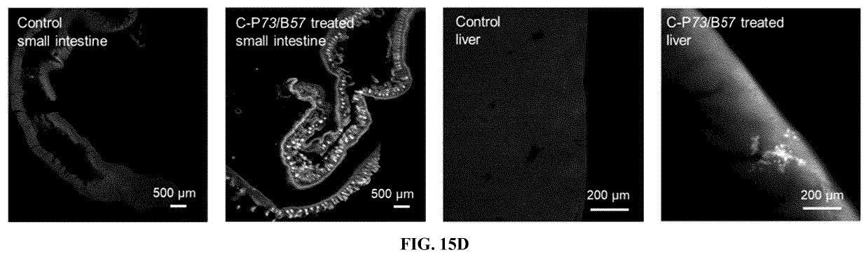

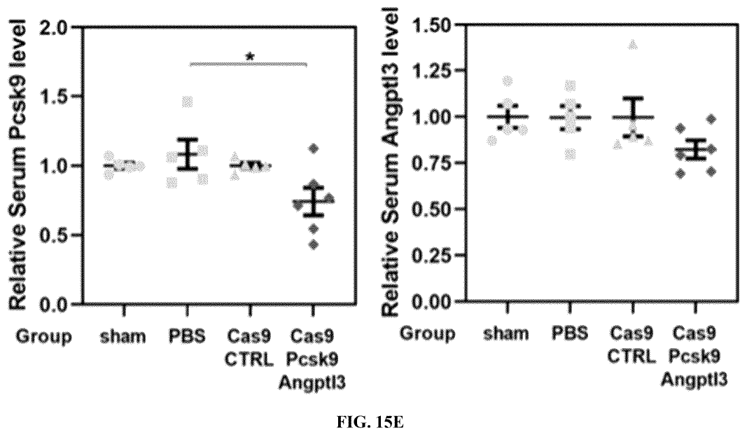

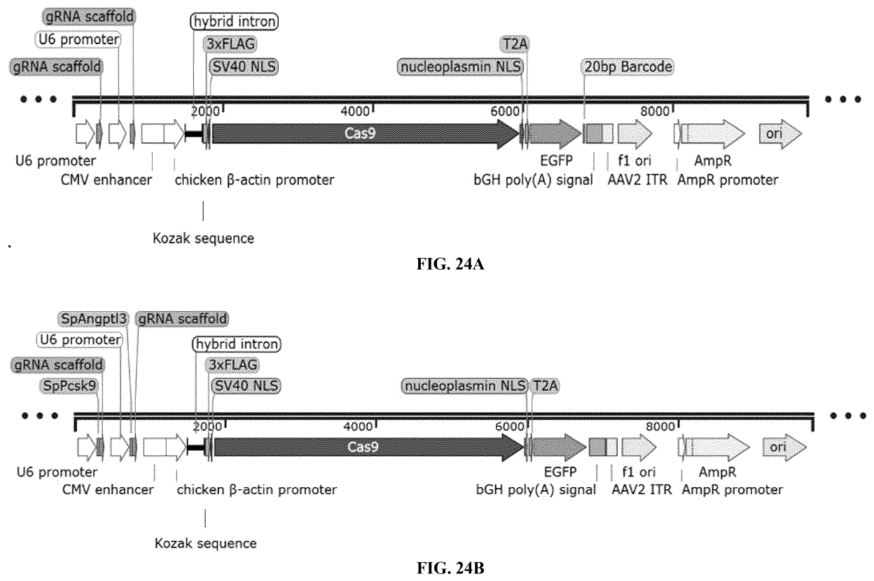

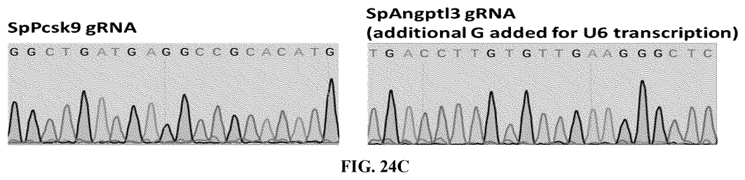

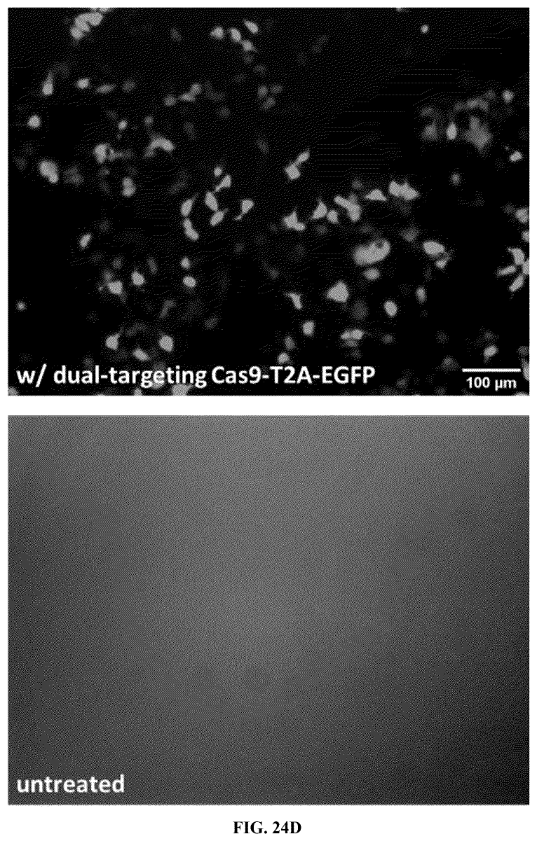

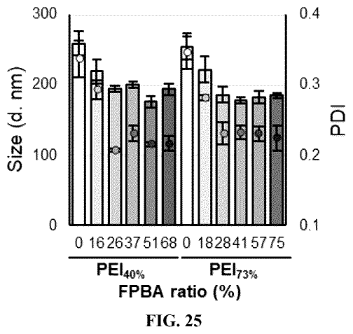

A CRISPR/Cas9 plasmid with a 20 bp barcode downstream at the Cas9-T2A-EGFP expression cassette was designed to test the oral formulation's ability to functionally deliver this gene-editing tool to cells. This plasmid also minimize the animal-to-animal and operation variations (FIGS. 24A-24C). We lyophilized each polyplex formulation with 3 wt % trehalose and mixed them after reconstitution prior to oral gavage. The size and PDI of the reconstituted polyplexes were comparable to those before lyophilization (FIGS. 13A and 25). Adult Balb/c mice were given orally with the barcoded polyplex mixture, naked barcoded plasmid mixture or PBS. At 24 h post-administration, total DNA was extracted from both muscularis externa, including the enteric nervous system and muscle (outer part) and intestinal mucosa/epithelium layer (inner part), followed by performing PCR to retrieve the barcode from the extracted DNAs. While at the outer part, we detected a similar level of the barcodes in both polyplex- and naked plasmid-treated groups, in the inner part, more barcoded plasmid from the polyplex-treated group was detected, indicating that polyplex formation helped the plasmid penetration (FIG. 15A). Further quantitative PCR analysis gave a similar trend; we saw a 3.5-fold higher amount of DNA barcodes in the inner part of the polyplex-treated mice, while there was no difference between groups in the outer part (FIG. 15B). We next sequenced the PCR product and found that the C-P₇₃/B₅₇ polyplex was dominant in the population (FIG. 15C). The inconsistency between in vitro and in vivo optimization is not uncommon as it has been reported. In this case, a higher FPBA ratio may be needed to stabilize the polyplex to overcome the harsh in vivo microenvironment, and the penetrated polyplexes may be therefore able to reach the liver or other organs through systemic circulation, although the formulation may be overstabilized for the in vitro transfection.

After identifying the composition for in vivo applications, we then tested whether the optimized polyplex (C-P₇₃/B₅₇) could be used for systemic editing through oral administration. We first used the LoxP-STOP-LoxP-TdTomato reporter mouse, A Ai14 (B6.Cg-Gt(ROSA)^{26Sortm14(CAG-tdTomato)Hze}/J), to confirm the polyplex could deliver the gene editing machinery to not only organs in the GI (e.g., intestines) but also other distal organs (e.g., liver) through systemic circulation following the traverse of tight junction in the intestines. When the Ai14 mice were given orally with the Cre-carrying C-P₇₃/B₅₇, we could detect TdTomato signals in both small intestine and liver tissues at 48 h post administration using the tissue clearing and Lightsheet imaging technique (FIG. 15D). This confirmed that the polyplex was able to cross the intestinal tight junction to reach the liver, and gene editing occurred as soon as 48 h after the oral dosing.

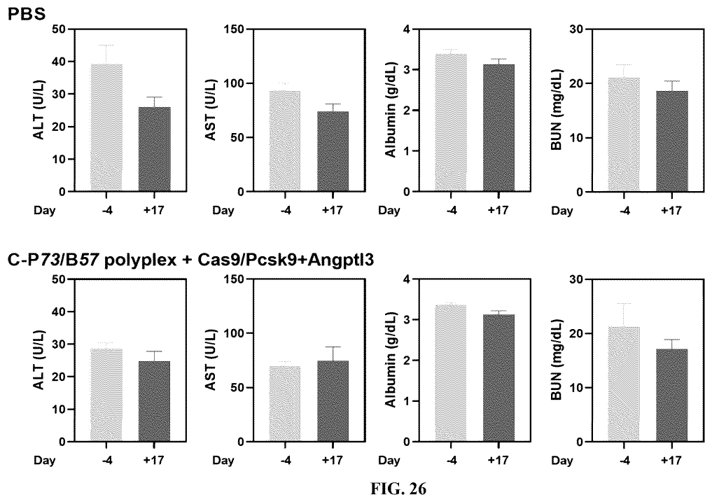

The feasibility of oral CRISPR delivery by targeting the two well-characterized liver targets, PCSK9 and ANGPTL3, which regulate the blood low-density lipoprotein and triglyceride levels, respectively was explored. The Pcsk9- and Angptl3-dual targeting Cas9/EGFP plasmid with the guides and confirmed by Sanger sequencing and in vitro transfection (FIGS. 24B-24D). For the in vivo validation, serum lipid and blood chemistry profiles were measured prior to the administration (at Day −4). Mice were then given orally with the C-P₇₃/B₅₇ polyplex encapsulating either the dual-targeting plasmid or the control plasmid (with two control guides) every other day for a total of three times. At Day 17 from the first administration, mouse sera and liver tissues were collected. Dual-editing by the C-P₇₃/B₅₇ polyplexes significantly reduced the mouse serum PCSK9 level (FIG. 15E; left) as well as the downstream lipid (FIG. 15F), without any adverse effect observed in blood clinical chemistry markers (FIG. 26). On the other hand, we also saw a similar trend in this group for the ANGPTL3 level, but the effect was moderate probably because of the location effect of Angptl3 guide's U6 expression cassette in the plasmid (FIG. 15E; right).

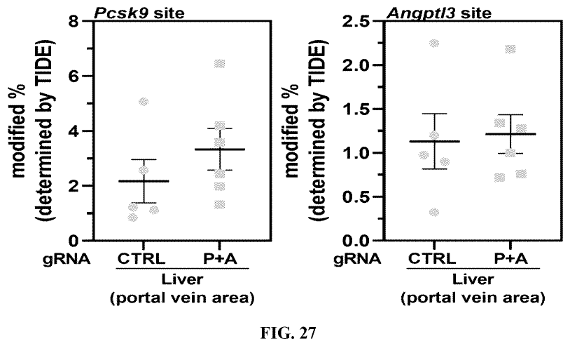

The editing performance at the genetic level was checked. However, unlike the protein and the downstream lipid levels, low indel events at the target loci in the genomic DNAs extracted from the liver tissues was detected (FIG. 27). This could be due to the sampling issue during the tissue processing or amplicon sequencing limitation. Dual-targeting Cas9 plasmid may create a large deletion in the treated mouse chromosome, which may not be easily detected by regular amplicon sequencing. Nevertheless, the use of the disclosed oral formulation is the first demonstration of feasible oral CRISPR delivery.

siRNA

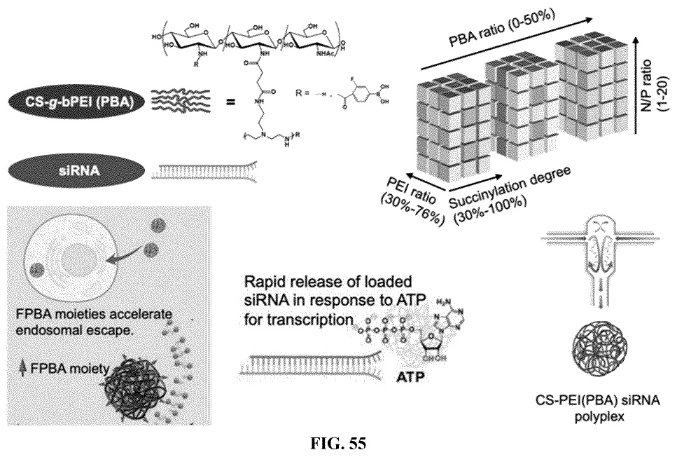

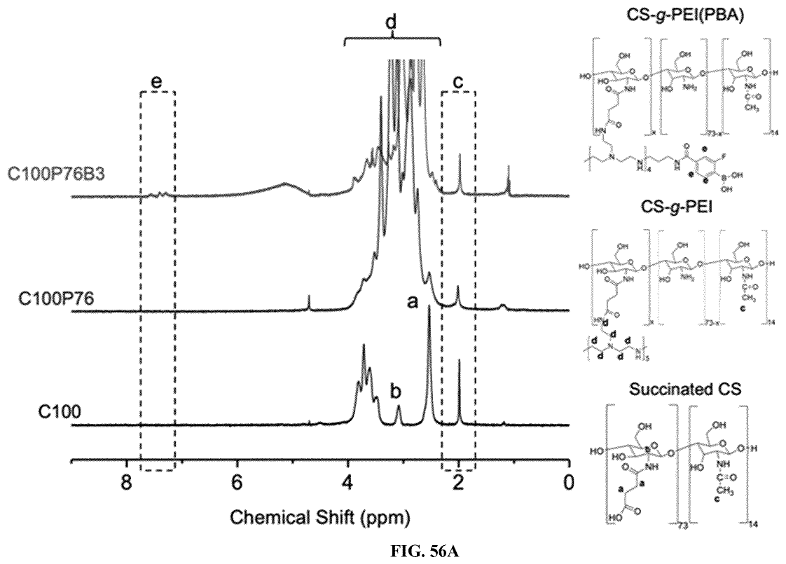

siRNA delivery presents distinct challenges. Upon reaching the cytosol, swift release of siRNA from endosomes into the cytoplasm is crucial. This is achieved due to the presence of PBA in the polymer. The integration of PBA represents enhances siRNA gene silencing efficiency. Hydrophobic interactions facilitated by PBA moieties stabilize the polyplex and safeguard encapsulated siRNA, particularly under acidic conditions. Subsequent intracellular release in target cells is facilitated by PBA, which enhances endosomal escape through hydrophobic interactions at late endosomal pH (pH 5.5), followed by binding to cytoplasmic adenosine triphosphate (ATP). This interaction weakens charged interactions of the polyplex, promoting unpacking or release of siRNA in the cytosol. A series of phenylboronic acid-functionalized chitosan-polyethyleneimine (CS-g-bPEI (PBA)) polymers tailored for siRNA delivery (FIG. 55) were developed.

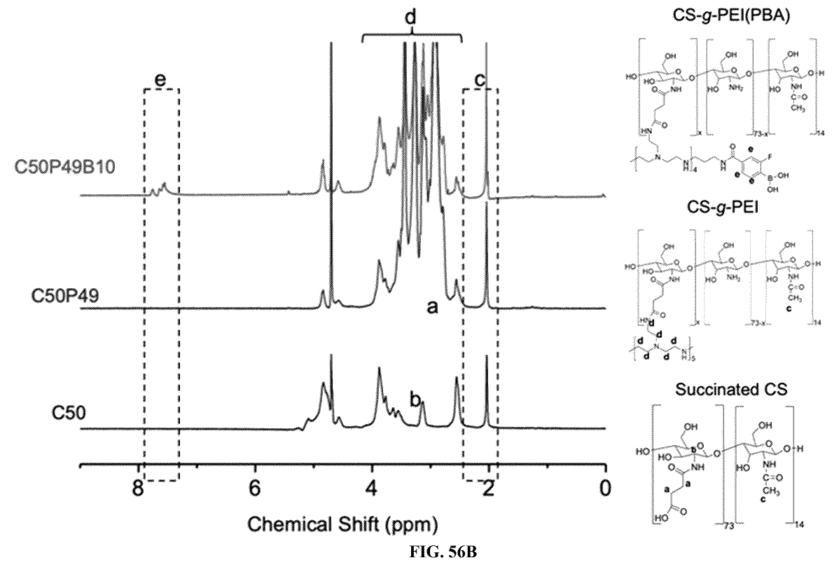

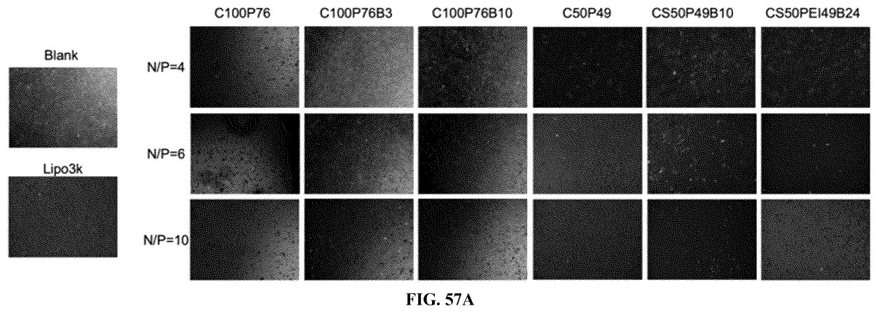

Two succinylated-CS variants, namely CS with succinylation degrees of 100% (C100) and 50% (C50), were used for to produce siRNA carriers for oral administration. First, low molecular weight PEI (MW=800) was grafted onto the succinylated-CS variants to produce PEI-grafted CS polymers (CPs). Then, the CPs are grafted with a PBA, such as 4-Carboxy-3 fluorophenylboronic acid (FPBA), to obtain CS-g-bPEI (PBA) polymers (CPBs). H1 NMR data confirmed the successful synthesis of C₁₀₀ and its derivatives, including CP with a PEI grafting ratio of 76% (C₁₀₀P₇₆) and CPB with a PEI grafting ratio of 76%, along with a PBA grafting ratio of 3% (C₁₀₀P₇₆B₃) (FIG. 56). Similarly, C₅₀ and its derivatives, such as CP with a PEI grafting ratio of 49% (C₅₀P₄₉) and CPB with a PEI grafting ratio of 49%, as well as a PBA grafting ratio of 10% (C₅₀P₄₉B₁₀), were synthesized and confirmed by H1 NMR data (FIG. 56). The siRNA is then encapsulated by the CP or CPB to result in an oral formulation for administering siRNA.

As shown in the Examples, the succinylation degree of chitosan, PEI grafting ratio, and PBA grafting ratio have been optimized to produce an siRNA carrier. This formulation represents a distinct advancement from the plasmid delivery carrier conventionally employed to administer siRNA. Thus, siRNA can successfully be delivered via oral administration through chitosan-based CS-g-bPEI polymers and its further phenylboronic acid-functionalized derivative, CS-g-bPEI (PBA). Their encapsulation of siRNA exhibit promising efficacy in inducing gene silencing in vitro, notably the C₁₀₀P₇₆ and C₅₀P₄₅B₂₄ polymers.

The concepts disclosed herein are not limited to the specific oral obesity medication, sarcopenia medication, gene editing shown herein. For example, it is specifically contemplated that the components included in oral formulations further comprise other components or combinations of component that can readily be formed into shaped objects and that are consistent with the intended oral administration of such compositions.

### EXAMPLES

### Example 1. Synthesis of FPBA-Functionalized CS-PEI Polymer

a. Materials for Preparation of Oral Formulation

Chitosan (CS; MW: 15,000 Da, DDA: 85%) was purchased from Polyscience Inc. (Warrington, PA). Methanol, acetone, branched-polyethyleneimine (b-PEI; Mw: 800 Da), 4fluorescamine, succinic anhydride, acetic acid, were purchased from Sigma-Aldrich® (St. Louis, MO). 4-Carboxy-3-fluorophenylboronic acid (FPBA) was purchased from Matrix Scientific (Columbia, SC). Tannic acid was purchased from Sigma-Aldrich® (St. Louis, MO).

b. Synthesis of Polymer

CS (200 mg) was reacted with succinic anhydride (160 and 480 mg) in 2% acetic acid (16 mL) for 1 h at room temperature. The mixture was neutralized using 1 M NaOHaq, followed by ethanol precipitation. Two succinated-CS products were collected (with 39% and 72% succination, determined by 1H-NMR spectra using D₂O with 2% CD₃COOD at room temperature or with 40% and 73% succination, determined by 1H-NMR spectra using D₂O with 2% CD₃COOD at room temperature). Afterwards, succinated-CS (100 mg) was conjugated with b-PEI (1.0 g) through amide coupling using EDC and NHS in distilled water, where the pH was adjusted to pH 5.5 using 5 M HClaq. After 24 h stirring, the mixture was dialyzed against 0.01 M HClaq for one day and dialyzed against distilled water for an additional day. The CS-PEI product was collected by freeze-drying.

FPBA moieties were then conjugated to the synthesized CS-PEI through amide coupling. CS-PEI (20 mg) was dissolved in 4 mL of 10 mM MES buffer (pH 5.0) containing 42 mg of D-sorbitol. FPBA (7.5 mg, 15 mg, 23 mg, 30 mg, and 38 mg for 39% b-PEI grafted CS-PEI; and 12 mg, 24 mg, 35 mg, 47 mg, and 59 mg for 72% b-PEI grafted CS-PEI) was dissolved in 4 mL of methanol. FBPA solution was slowly added to the CS-PEI solution in a dropwise manner. The addition process commences with the FPBA solution featuring the lowest acid concentration, followed by a gradual increase in concentration for subsequent solutions. This progressive addition of FPBA directly influences the introduction ratio, with higher FPBA concentrations generally resulting in an increased ratio and, conversely, lower concentrations leading to a reduced ratio. After the five concentrations FBPA solutions have been added, the mixture is further added 85 mg of DMT-MM. The reaction mixture was stirred for 24 h at room temperature. The reacted solution was immediately dialyzed against 0.01 M HClaq for one day and dialyzed against distilled water for an additional day. The final product was then collected by freeze-drying.

The FPBA introduction ratio into CS-PEI was determined by 1H-NMR spectra (D₂O at room temperature) and the fluorescamine method. Notably, for the calculation of FPBA introduction ratio, the amount of primary amine on b-PEI was estimated to 42%, based on published literature.

### Example 2. Synthesis of LCN2 Encapsulated with FPBA-Functionalized CS-PEI Polymer

The synthesized CS-PEI (FPBA) polymer was dissolved in 10 mM PB buffer (pH 5.0) containing 10 mM D-sorbitol (pH 7.3). Dissolution was achieved by sonication at 40° C. for 1 hour, followed by vortexing overnight at room temperature. The resulting polymer solutions were filtered through a 0.22 μm nylon filter. Separately, LCN2 was diluted in 10 mM PB buffer. In certain formulations, tannic acid (TA) was introduced to enhance the complexation between the polymer and LCN2, leading to the formation of nanoparticles. Specifically, 50 μL of 0.2 mg/mL LCN2 protein solution was mixed with 50 μL of 0.04 mg/mL TA solution and incubated for 15 minutes. Subsequently, 100 μL of 1,000 μg/mL CS-PEI (FPBA) polymer solution was added to the mixture, followed by an additional incubation for 15 minutes.

Nanoparticles were prepared by mixing 2-unit volume of CS-PEI (FPBA) solution and 1-unit volume of the LCN2 solution under vortex at a complexing ratio of 15:1. In specific formulations, the generated nanoparticles were further mixed with a hyaluronic acid (HA) solution. Nanoparticles were then incubated at 4° C. overnight before use. To ensure quality control, the nanoparticle formation was verified by dynamic light scattering (DLS) measurement using ZetaSizer® NanoZS90 (Malvern, UK) at 25° C. for quality control.

### Example 3. Synthesis and Evaluation of FPBA-Functionalized CS-PEI Polymer for Encapsulation of Plasmids

For oral administration of plasmids, CS-PEI (MW of CS and b-PEI: 15 kDa and 800 Da, respectively) at the b-PEI grafting ratios of 40% and 73% were synthesized, followed by introducing PBA moieties with various ratios through amide coupling. Both the 1H NMR spectrum and fluorescamine analysis revealed the conversion ratio of primary amino groups into FPBA at 16%, 26%, 37%, 51%, and 68% for the 40% b-PEI-grafted CS, and 18%, 28%, 41%, 57%, and 75% for the 73% b-PEI-grafted one (FIG. 5). In total, a library of 12 FPBA-functionalized CS-PEI was prepared (see Table 1, denoted as CS-PEI_{X%}(FPBA)_{Y%}, where X and Y represent the b-PEI grafting and the FPBA introduction ratios, respectively).

TABLE 1
Introduction ratio of FPBA moieties into CS-PEI

FPBA introduction ratio (%)

| | | | | | | |
|---|---|---|---|---|---|---|
| PEI 40% | 0 | 16 | 26 | 37 | 51 | 68 |
| PEI 73% | 0 | 18 | 28 | 41 | 57 | 75 |

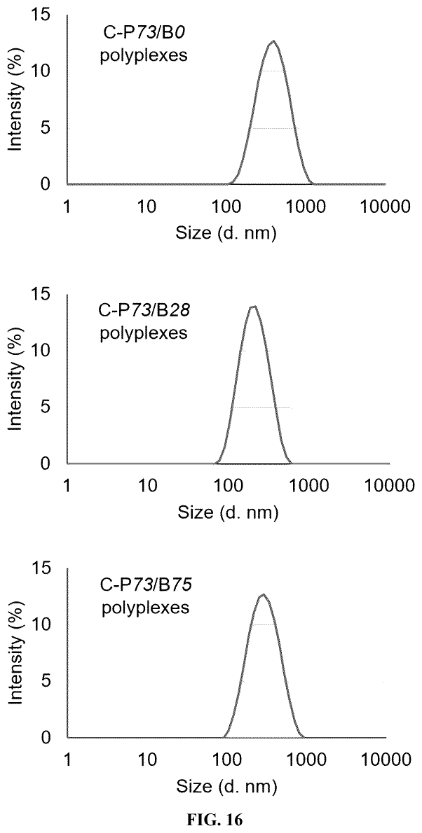

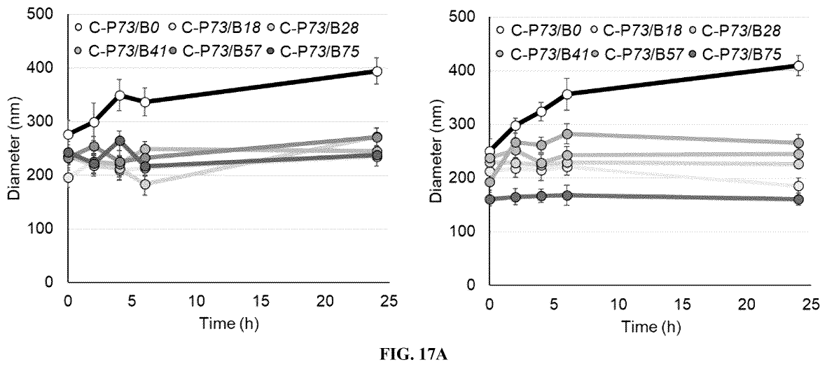

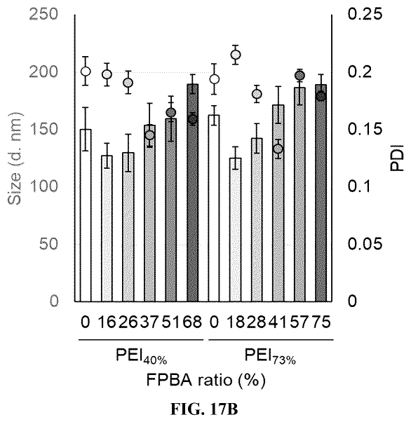

Polyplexes, prepared from CS-PEI_{X%}(FPBA)_{Y%} and plasmid (termed C-P_{X}/B_{Y}) at a residual molar ratio of all amino groups on CS-PEI_{X%}(FPBA)_{Y%} to phosphate groups on plasmid (N/P ratio) of 60, achieved efficient in vitro transfection with low cytotoxicity. Dynamic light scattering (DLS) measurements showed a diameter range of 153-207 nm with a relatively narrow polydispersity index (PDI; FIGS. 13A and 16). The size of C-P_{X}/B_{Y} polyplexes (Y≠0) was maintained for at least 24 h at 37° C., but C-P_{X}/B₀ polyplexes significantly aggregated over time (FIG. 17A), indicating that FPBA modification enhanced the colloidal stability of the polyplex structure even at pH 7.4. FPBA functionalization decreased the ζ-potential of C-P_{X}/B₀ polyplex from 38 mV to 19-25 mV at pH 7.4. At the lower pH (1.5), which mimicked the stomach microenvironment, no changes in the polyplexes' physicochemical characteristics were observed (FIG. 17B).

### Example 4. Synthesis of CRISPR/Cas System Encapsulated with FPBA-Functionalized CS-PEI Polymer

Synthesized CS-PEI (FPBA) was dissolved in 10 mM MES buffer (pH 5.0) containing 15 mM D-sorbitol by sonication at 40° C. for 1 h and then by vortex overnight at room temperature. These polymer solutions were filtered using a 0.45 μm nylon filter. The plasmid was dissolved in 10 mM HEPES buffer (pH 8.2). The concentration of CS-PEI (FPBA) was determined based on the residual molar concentration of all amino groups on CS-PEI (FPBA) [N] and phosphate groups on plasmid [P] (N/P ratio). Polyplexes were prepared by mixing 1-unit volume of CS-PEI (FPBA) solution and 2-unit volume of the plasmid solution under vortex at an N/P ratio of 60. Polyplexes were then incubated at 4° C. overnight before use. The polyplex formation was verified by dynamic light scattering (DLS) measurement using ZetaSizer® NanoZS90 (Malvern, UK) at 25° C. for quality control.

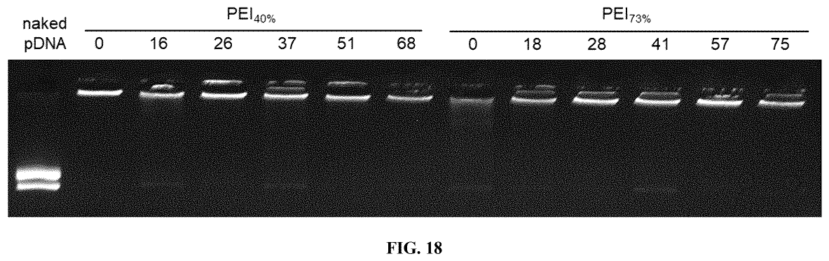

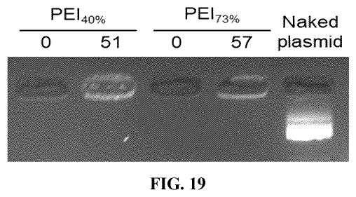

To study whether FPBA would stabilize the polyplex through hydrophobic interactions, the polyplexes were incubated with a competitive polyanion, dextran sulfate, at an anion/phosphate ratio of 30. Electropherograms showed that dextran sulfate completely displaced the plasmid in the polyplexes with low FPBA ratios (C-P_{X}/B₀, C-P₄₀/B₁₆, and C-P₇₃/B₁₈; FIG. 13B), but not the ones with an FPBA ratio above 37%. Similarly, in the acidic environment (pH 1.5), there was no plasmid release regardless of the FPBA ratio in the presence of dextran sulfate (FIG. 18). This could be derived from both the increased charge density of the polyplexes through protonation of the secondary and tertiary amines on grafted b-PEI and the hydrophobic interactions between PBA moieties. In the polyplexes with low and without FPBA modification, the increased charge density can stabilize the polyplex structure at pH 1.5. The polyplexes with high FPBA modification, although they had less protonatable amines due to the FPBA introduction, also performed high stability at pH 1.5, which could not be explained by only increased charge density. Possibly, the hydrophobic interaction between FPBA moieties under acidic condition contributed to the stabilization effect. In addition, incubation with bile salt, a common component in the stomach that may destabilize polyplexes, did not trigger plasmid release (FIG. 19). Altogether, these results support the use of the polyplex for oral gene delivery.

Förster resonance energy transfer (FRET) technique was applied to evaluate the plasmid condensation by double-labeling the plasmid with Cy3 and Cy5 dyes. At pH 7.4, no correlation was observed between FRET efficiency and the FPBA ratio due to the trade-off between tight packaging by FPBA hydrophobic interactions and reduced charge density by negatively charged FPBA (FIG. 13C). In contrast, the FRET efficiency at pH 1.5 strongly correlated with the FPBA ratio, because all the FPBA moieties assumed the uncharged hydrophobic structure, again confirming the hypothesis that FPBA modification could protect the plasmid better at a low pH microenvironment.

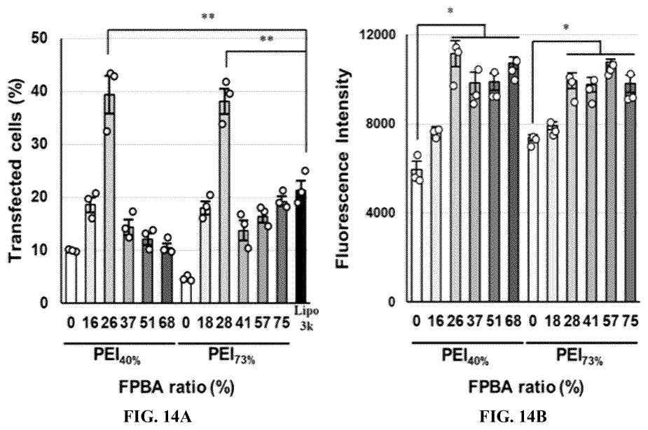

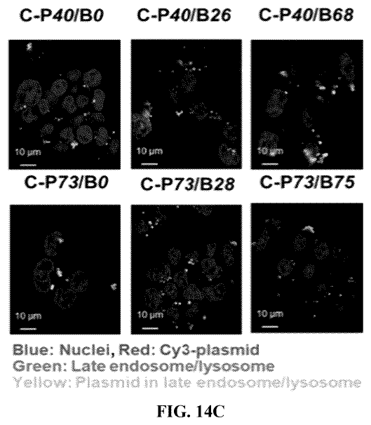

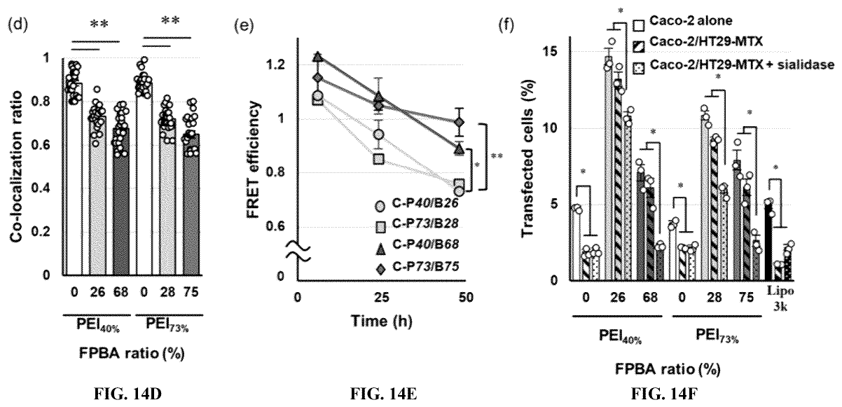

Transfection efficiency of the C-P_{X}/B_{Y} polyplexes was evaluated in the human colorectal line, HCT116, with the GFP-encoding plasmid. C-P₄₀/B₂₆ and C-P₇₃/B₂₈ performed better than Lipofectamine™ 3000 in GFP transfection in this line (FIG. 14A). Surprisingly, the transfection efficiency was inconsistent with the polyplex stability (FIG. 13B).

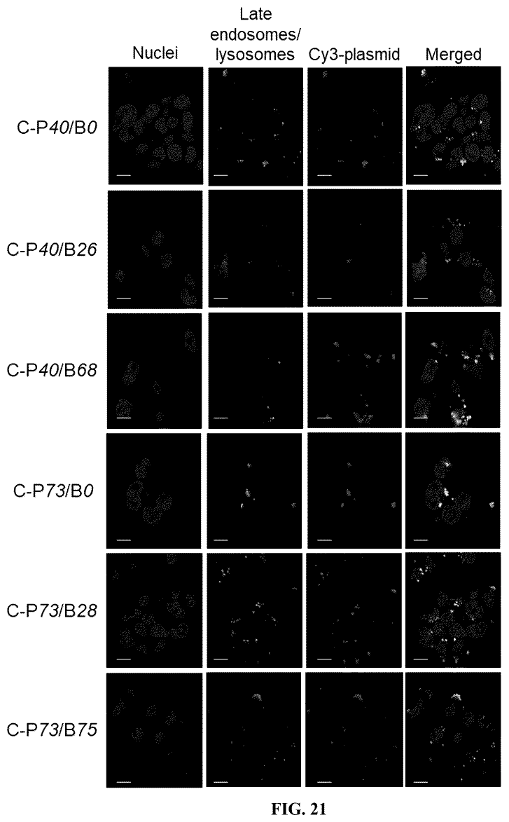

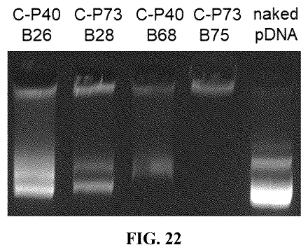

To understand why transfection efficiency does not seem correlated with polyplex stability, we investigated the cellular uptake, endosomal escape ability, and intracellular plasmid release. FPBA introduction helped the internalization of polyplexes up to an FPBA ratio of 26% (FIG. 14B). Confocal microscopy revealed that the FPBA-functionalized polyplexes were more efficient in endosomal escape than their control counterparts (C-P_{X}/B₀), but no significant difference was observed among the groups with FPBA modification (FIGS. 14C, 14D, and 21). In contrast, when using the double-labeled plasmid to check the ATP-triggered release through the formation of phenylboronate ester linkage between ATP and FPBA moieties, the polyplexes with the better transfection performance (C-P₄₀/B₂₆ and C-P₇₃/B₂₈) had faster FRET reduction than the ones with higher FPBA ratios (C-P₄₀/B₆₈ and C-P₇₃/B₇₅; FIG. 14E), indicating more efficient plasmid release from C-P₄₀/B₂₆ and C-P₇₃/B₂₈. Probably, excess FPBA modification may be overstabilized the polyplex and hinder the plasmid release in vitro, as the C-P₄₀/B₆₈ and C-P₇₃/B₇₅ polyplexes showed higher stability against polyanions even in the presence of ATPs (FIG. 22). The moderately stabilized polyplexes, C-P₄₀/B₂₆ and C-P₇₃/B₂₈, therefore showed the best in vitro transfection efficiency among all the polymer candidates.

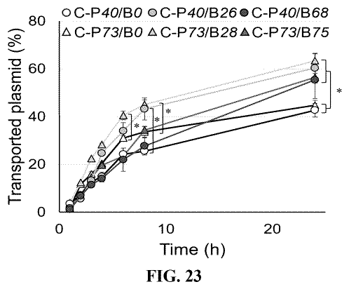

As the FPBA could act as a ligand against the sialic acid on mucin, we compared its transfection efficiency in mucosa+ Caco-2/HT29-MTX coculture versus Caco-2 alone (no mucosa). In the no mucosa condition, similar to the transfection in the HCT116 line (FIG. 14A), C-P₄₀/B₂₆ and C-P₇₃/B₂₈ showed the best efficiency (FIG. 14F, open bar). In the coculture condition, the mucosal barrier did not compromise the transfection efficiency of FPBA-functionalized polyplexes, while the control groups (C-P_{X}/B₀ and Lipofectamine™ 3000) lost the transfection potency significantly (FIG. 14F, hatched bar). To confirm the contribution of FPBA-sialic acid interaction, we further added sialidase to block it. As expected, sialidase significantly reduced the transfection efficiency of FPBA-functionalized polyplexes but did not affect the control groups (FIG. 14F, dotted bar), indicating that FPBA-sialic acid interaction was important for polyplexes to pass through the mucosal barrier. Transport of the polyplexes through the mucus layer was also evaluated using a mucus-coated transwell diffusion assay. FPBA modification allowed more polyplexes to pass through the mucus barrier after 24 h incubation when compared with their parental C-P_{X}/B₀ counterparts (FIG. 23). This result confirmed the contribution of FPBA moieties in mucus penetration and was consistent with the transfection result using the Caco-2/HT29-MTX coculture condition. Interestingly, the transport of C-P₄₀/B₂₆ and C-P₇₃/B₂₈ was more efficient at the early time points (6-8 h), but all the FPBA-decorated polyplexes (C-P₄₀/B₂₆, C-P₇₃/B₂₈, C-P₄₀/B₆₈, and C-P₇₃/B₇₅) reached a similar level of deposition at the end point (24 h). The differences in kinetics could be due to the stronger interaction between FPBA and mucin through multivalent bonds, which led to slower diffusion of polyplexes in the mucus layer.

### Example 5. Evaluation of Oral Formulation for CRISPR/Cas System

a. Dual-Targeting Cas9-T2A-EGFP Plasmid Construction

The dual gRNA-encoding Cas9-T2A-EGFP plasmids used in this study for in vivo polyplex screening and oral CRISPR validation were built based on the PX333/Cas9 vector backbone (Addgene® #64073; Watertown, MA). The T2A-EGFP sequence was first retrieved from another Cas9-T2A-EGFP plasmid (Addgene® #48138) by PCR using Platinum SuperFi high-fidelity polymerase (Thermo-Fisher®) with the following two specific primers:

Forward (SEQ ID NO. 3):
GAAAAAGGCCGGCCAGGCAAAAAAGAAAAAGTTATTCGGCAGTGGA
Reverse (SEQ ID NO. 4):
TAGTTAGAATTCTTACTTGTACAGCTCGTCCATGCCGAGAGTGAT

Afterwards, both PX333 vector and the T2A-EGFP amplicon were digested by FseI and EcoRI-HF (New England Biolabs®, Ipswich, MA), and subsequently ligated using T7 ligase (New England Biolabs®) at 25° C. for 30 mins. The cloned product was then transformed into Stellar™ competent cell (Takara, Japan). The dual gRNA-encoding Cas9-T2A-EGFP plasmid was purified using NucleoBond® Xtra Midi Plus Endotoxin-Free kit (Macherey-Nagel, Germany). The plasmid sequence was verified by Sanger sequencing (Genewiz, South Plainfield, NJ).

For the barcode plasmid used in the in vivo polyplex screening, a 20 bp barcode was further cloned into this plasmid by Gibson assembly at the EcoRI cut site. For Pcsk9- and Angptl3-targeting, the two gRNAs that we optimized in our previously published work⁵ were cloned into the plasmid using the BbsI and BsaI restriction enzymes (New England Biolabs®) by following previously established protocol, respectively. The plasmids used in this study are shared in Addgene®.

b. Gel Electrophoresis

Polyplexes were incubated with dextran sulfate in 150 mM NaCl solution adjusted to pH 7.4 or 1.5. The residual charge ratio of sulfate in dextran sulfate (A) to phosphate in plasmid (P) was set to 30 (A/P ratio of 30, final plasmid and dextran sulfate concentration were 25 and 385 μg/mL, respectively). After 1 h incubation, 15 μL of the mixture with 1.67 μL of 10×DNA loading buffer was loaded onto 1 wt % agarose gel pre-stained with ethidium bromide (0.5 mg/mL). In addition, polyplexes were incubated in the presence of dextran sulfate (A/P ratio of 10) and ATP in a concentration that matched the intracellular level at pH 7.4. The release profile of encapsulated plasmid in a condition containing ATP (3 mM) or bile salt (20 mM) was evaluated by gel electrophoresis through the same procedure.

c. Förster Resonance Energy Transfer (FRET) Measurement

Plasmid was double-labeled using Label IT™ Cy3 and Cy5 labeling kits (Mirus Bio, Madison, WI) by following established protocol. Polyplexes encapsulating Cy3/Cy5 double-labeled plasmid were then prepared as aforementioned. Labeled polyplexes were incubated in 150 mM NaCl solution adjusted to pH 7.4 or 1.5 for 10 mins. The fluorescence intensity of each sample was measured by FLUOstar® OPTIMA plate reader (BMG Labtech®, Germany). The excitation wavelength was set to 485 nm, and the emission wavelengths were set to 570 nm for Cy3 and 670 nm for Cy5. The FRET efficiency was calculated as follows:

$$FRET efficiency = \frac{Cy 5 fluorescence intensity}{Cy 3 fluorescence intensity}$$

d. Cell Culture

HCT116 cell was cultured in McCoy's 5A containing 10% FBS and 1× penicillin/streptomycin (100 U penicillin and 100 μg streptomycin). Caco-2 and HT29-MTX cells were cultured in DMEM containing 10% FBS, 1×MEM non-essential amino acid, 1 mM sodium pyruvate and 1× penicillin/streptomycin. All the cells were cultured in a humidified atmosphere with 5% CO₂ at 37° C.

e. Cellular Uptake of Polyplexes

HCT116 cells were seeded on a 12-well plate at 50,000 cells/well and cultured for 24 h. After the medium was replaced with the fresh one, polyplex solution containing 2.5 μg of Cy5-labeled plasmid was added to each well. At 24 h post-treatment, the cells were washed by DPBS twice and harvested. The Cy5 fluorescence of collected samples was evaluated using Fortessa™ flow cytometer (BD™, Franklin Lakes, NJ) with a 633 nm laser and 670/30 bandpass filter.

f. In Vitro Transfection

HCT116 cells were seeded on a 12-well plate at 50,000 cells/well at 24 h prior to transfection. On the day of transfection, the culture medium was replaced with the fresh one, the polyplex solution containing 2.5 μg of EGFP or Cas9-T2A-EGFP plasmid was added to each well. At 24 h post-transfection, culture medium was replaced again, and the cells were incubated for another 24 h. Transfection efficiency was determined by FACS. Briefly, cells were first washed with DPBS twice and then harvested. Percent of EGFP+ cells was determined using BD™ Fortessa™ Cell Analyzer using 488 nm laser excitation with the 530/30 bandpass filter and 505 LP dichroic filter.

g. In Vitro Cytotoxicity Assay

HCT116 cells were seeded on a 96-well plate at 5,000 cells/well at 24 h prior to transfection. On the day of transfection, the culture medium was replaced with the fresh one, the polyplex solution containing 250 ng of Cas9-T2A-EGFP plasmid was added to each well. At 24 h post-transfection, 10 μL of CCK-8 solution was added to each well, followed by incubating for 2 h. The absorbance of each sample at 450 nm was measured by FLUOstar® OPTIMA plate reader.

h. Confocal Laser Scanning Microscopy Measurement to Visualize the Endosomal Escape of Polyplexes

HCT116 cells were seeded on a 35-mm glass-bottom dish with 2 mL of culture medium (100,000 cells/dish). After 24 h incubation, the culture medium was replaced with the fresh one (1 mL), and 150 μL of polyplex solution containing 5 μg of Cy3-labeled plasmid was added. After a 24 h incubation, the treated cells were washed by DPBS twice and stained by Hoechst 33342 and Lysotracker Green for visualization of cell nuclei and for late endosomes/lysosomes, respectively. CLSM images were obtained using Ti Eclipse inverted microscope (Nikon, Japan) with a 63× oil objective at 405 nm (for Hoechst 33342), 488 nm (for Lysotracker Green) or 561 nm excitation (for Cy3-labeled plasmid). The co-localization ratio of Cy3-labeled plasmid in late endosome/lysosome was calculated from at least 25 individual cells:

$$Co - localization ratio = \frac{Number of Cy 3 pixels with late endosomes and lysosomes}{Number of all observed Cy 3 pixels}$$

i. Intracellular FRET Measurement

HCT116 cells were seeded on a 6-well plate at a cell density of 100,000 cells/well (2 mL) and incubated for 24 h. The culture medium was then replaced with the fresh one (1 mL), and 150 μL of polyplex solutions containing 5 μg of Cy3/Cy5 dual-labeled plasmid was added. After 6 h incubation, the cells were washed twice with DPBS to remove the remaining extracellular polyplexes. The cells were harvested by trypsinization at different time points (6, 24, and 48 h post-transfection). The Cy3 and Cy5 fluorescent signals of collected samples were measured using BD™ Fortessa™ Cell Analyzer with 561 nm excitation and 582/12 nm bandpass filter for Cy3 and 670/30 nm bandpass filter plus 635 LP dichroic filter for Cy5. The FRET efficiency was calculated as aforementioned.

j. Transfection in the Caco-2/HT29-MTX Coculture Model

Caco-2 alone or a mixture of Caco-2 and MT29-MTX (Caco-2: HT29-MTX=4:1) was seeded on a 24-well plate at a cell density of 12,500 cells/well. The cells were cultured for 7 days with medium replacement every other day for mucus layer generation, followed by the treatment of α2,3-sialidase (final concentration: 6.25 mU/mL; Takara, Japan) or DPBS for 1 h prior to transfection. Polyplexes containing 2 μg of EGFP-encoding plasmid were added to each well. At 24 h post-transfection, the culture medium was replaced again, and the cells were incubated for another 24 h. The cells were washed by DPBS twice and then harvested. EGFP signals of collected samples was measured as described above.

k. Transport Validation of C-P_{X}/B_{Y} Through Mucus Layer

Mucin from porcine stomach Type II (Sigma™) was dissolved in 1 M NaOH at 20 mg/mL and diluted by D-PBS to 5 mg/mL. The pH was adjusted to 7.4 using 0.1 M HCl_{aq}. The mucin solution (50 μL) was added to the upper chamber of 24-well transwell plates (3 μm pore size, Costar Corning®, Cambridge, MA), and 600 μL of DPBS was added to the lower chambers. The plates were incubated overnight at 4° C. Before use, the plates were incubated at 37° C. for 1 h, and then 200 μL of Cy5-plasmid loading polyplex solution was added to each upper chamber. After incubation at 37° C., the fluorescence intensity of the supernatant in the lower chamber was measured using FLUOstar® OPTIMA plate reader (set to 633 nm for the excitation and 670 nm for the emission).

l. In Vivo Optimization of C-PX/BY Formulation for Oral CRISPR Delivery

The Cas9-T2A-EGFP plasmid with a unique 20 bp barcode (20 μg) was complexed with each polyplex formulation in 10 mM MES buffer (3.6 mL) at pH 5.0 at a N/P ratio of 60. After overnight incubation at 4° C., the polyplex solution was mixed with trehalose solution at a final trehalose concentration of 3 wt %, followed by freeze-drying. The polyplex was reconstituted using 120 μL UltraPure Water (Thermo-Fisher®) by vertexing prior to use. Afterwards, the polyplexes were pooled. Prior to the administration, adult female Balb/c mice (10-week-old, JAX® Lab, Bar Harbor, ME) were under fasting for overnight. The mixture containing all 12 polyplex formulations (total plasmid amount equivalent to 240 μg in 200 μL) was then given to mice through oral gavage. The lower small intestine was collected at 24 h post-administration. The mucosa and inner part of small intestine were separated under the microscope, and the tissues were kept in 1×DNA/RNA Shield solution (Zymo Research, Irvine, CA) to minimize degradation. After homogenization, total DNA from both mucosa and the inner parts of small intestine were extracted using Zymo Research Quick-DNA/RNA Miniprep Plus kit. The barcode sequences in the total DNA were then amplified by PCR using Takara Terra™ Direct PCR polymerase. The amplified products were then ligated into a PUC19 plasmid and transformed into Takara Stellar™ competent cell. The sequence information was retrieved by colony sequencing (Genewiz).

m. In Vivo Oral CRE Delivery in Ai14 Mice

Adult Ai14 mice (B₆.Cg-Gt(ROSA)26Sor^{tm14(CAG-tdTomato)Hze}/J, JAX® Lab) were given orally with our optimized C-P₇₃/B₅₃ polyplex carrying the CRE/GFP plasmid (generous gift from Dr. Alejandro Chavez at UCSD; 600 μg). At 48 h post-administration, both small intestine and liver were dissected. The harvested tissues were fixed with 4% PFA overnight and cleared with the FDISCO method as previously described. Briefly, the tissues underwent a sequential dehydration process with 50%, 70%, 80% and 100% THF. The tissues were subsequentially immerged in DBE with 0.5 v/v % vitamin E. The tissues were imaged using the UltraMicroscope® II Light Sheet Microscope at Columbia-Zuckerman Institute.

n. In Vivo Oral CRISPR Delivery of C-P73/B53 Polyplex

To validate the oral CRISPR delivery performance of the C-P₇₃/B₅₃ polyplex, another in vivo experiment to target Pcsk9 and Angptl3 was carried out. Prior to the administration (Day −4), the blood of adult female Balb/c mice was collected through the submandibular vein, and a panel of serum chemistry (albumin, alanine aminotransferase, aspartate aminotransferase, blood urea nitrogen, creatinine, and lipid profile) was analyzed by IDEXX (North Grafton, MA). The polyplex was prepared by C-P₇₃/B₅₃ and Pcsk9-/Aangptl3-dual targeting Cas9-T2A-EGFP plasmid. Another polyplex was prepared by the same polymer but the Cas9-T2A-EGFP plasmid with control gRNAs as a control. Polyplex solution was lyophilized with the same procedure and stored at 4° C. before use. The lyophilized polyplexes containing 200 μg of plasmid were completely resuspended in 200 μL of UltraPure Water. The concentrated polyplex solution (200 μL) was given to mice orally three times every other day (total 600 μg of plasmid given). At the endpoint (Day 17 after the first administration), mouse serum was collected again, and then both liver and small intestine were dissected. Serum PCSK9 and ANGPTL3 levels were measured by the Mouse PCSK9 ELISA kit (Abcam®, Cambridge, MA) and ANGPTL3 Mouse ELISA kit (Thermo-Fisher®), respectively. The serum samples were also sent to IDEXX for the same serum chemistry panel analysis. To quantify the gene editing efficiency, genomic DNAs from the liver and the small intestine close to ileum were extracted using Zymo Research Quick-DNA™ Miniprep Plus kit. The target loci were then amplified Takara Terra™ Direct PCR polymerase with 100 μg of the genomic DNA. The PCR product was then purified and quantified by PicoGreen® assay. Gene editing efficiency was determined by sequencing (Genewiz) with TIDE analysis.

o. Statistical Analysis

Data are presented as mean±standard error of mean (SEM). Sample size was described in the caption of each figure. Statistical difference in each experiment was determined by following methods:

FIG. 14A: one-way ANOVA with Dunnett's post-hoc test (compared with the Lipofectamine 3000 group);

FIGS. 14B and 14D: one-way ANOVA with Dunnett's post-hoc test (compared with the C-P40/B0 or C-P73/B0 group);

FIG. 14E: unpaired, 2-tailed Student's T-test;

FIG. 14F: one-way ANOVA with by Dunnett's post-hoc test (compared with the transfection efficiency in the Caco-2 alone condition);

FIG. 15D: one-way ANOVA with Tukey's post-hoc multiple comparisons;

FIG. 15E: paired, 2-tailed Student's T-test.

In addition to a described implementations, other implementations are also contemplated and possible. These listed here, and many others, will become readily apparent from this disclosure.

It will be understood that implementations of the oral obesity medication include but are not limited to the specific components disclosed herein, as virtually any components consistent with the intended operation of various oral obesity medication may be utilized. Accordingly, for example, it should be understood that, while the drawings and accompanying text show and describe particular oral obesity medication implementations, any such implementation may comprise any shape, size, style, type, model, version, class, grade, measurement, concentration, material, weight, quantity, and/or the like consistent with the intended operation of oral obesity medication.

### Example 6. Delivery of CRISPR-Cas9/dCas9 Elements

The results in this example demonstrate the disclosed oral formulation successfully innovative orally-administers gene editing system without the use of viral carriers. This opens exciting new avenues for addressing the demands of both localized gene editing in the gastrointestinal (GI) tract and systemic gene editing in a patient-friendly, non-invasive, and repeatable manner.

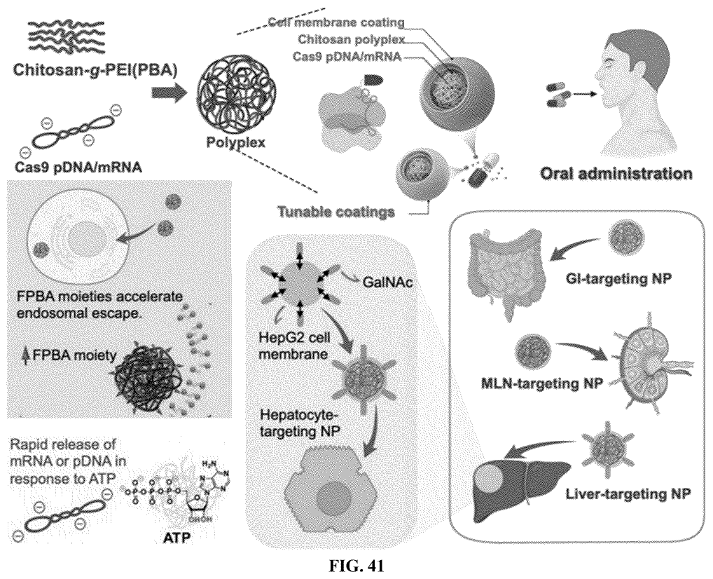

The programmable oral gene editing system consists of three major components: PEI-grafted chitosan modified with phenyboronic acid (PBA), Cas9 mRNA or pDNA, and coatings of the polyplex using eukaryotic cell membrane fragments. Upon oral delivery, gene editing can be targeted to the gastrointestinal tract, mesenteric lymph node, and liver. Programmability is achieved at different levels: coating materials at the tissue level, targeting ligands such as N-acetylgalactosamine (GalNac) at the cell level, and interactions between ATP and PBA at the intracellular level. Our preliminary results demonstrate the feasibility of this versatile oral gene editing system for editing the three proposed tissues. FIG. 41 describes the design of the oral gene delivery system.

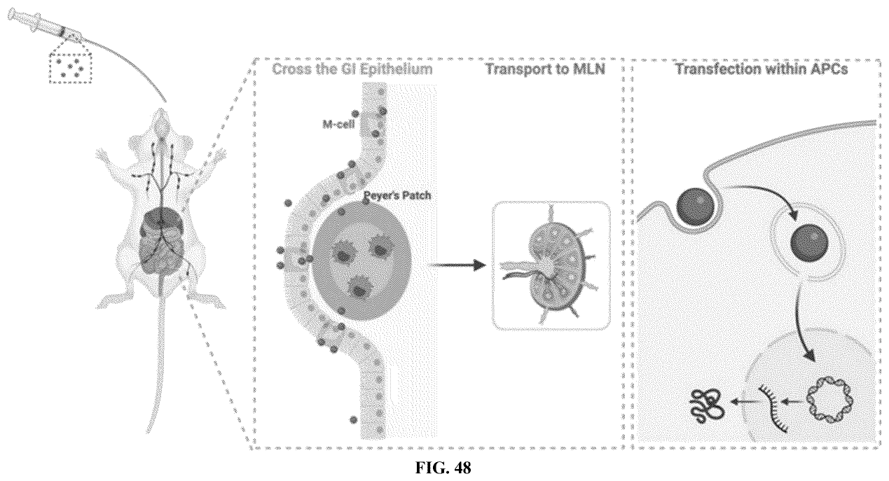

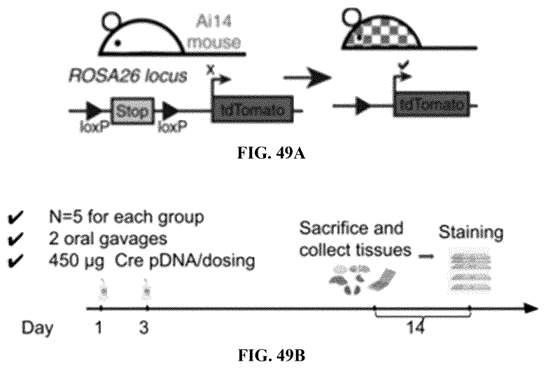

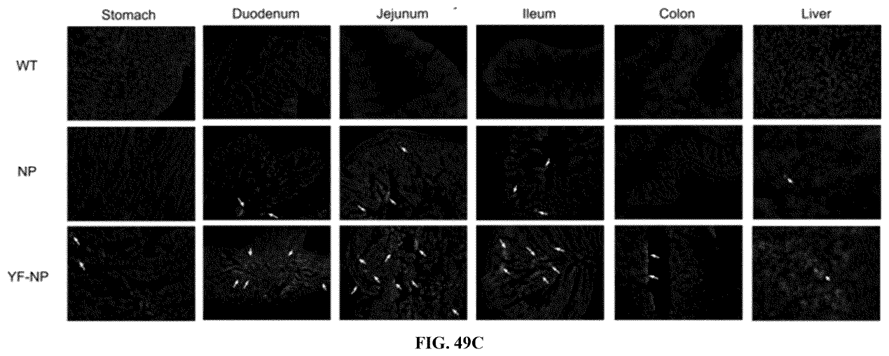

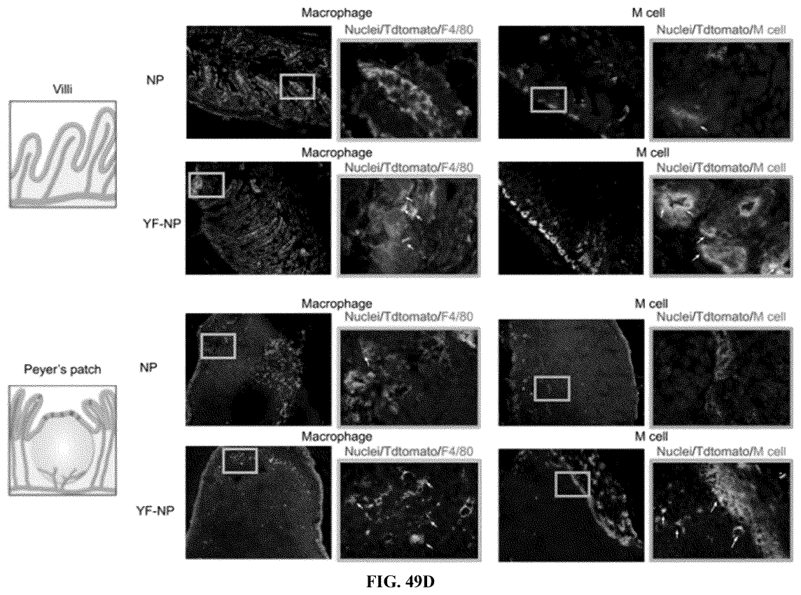

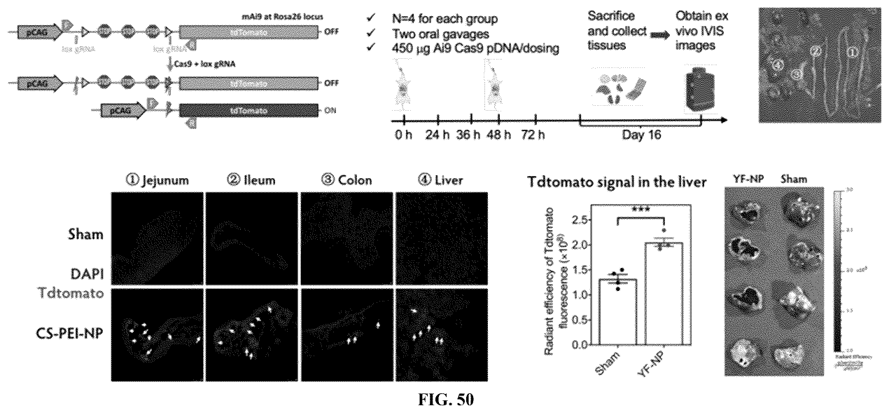

Previously, we found that a single oral dose of chitosan-grafted-branched polyethyleneimine nanoparticles containing an insulin-encoding plasmid provided hyperglycemic protection for more than 10 days in a diabetic mouse model. We further optimized this gene carrier for oral gene editing by modifying with phenylboronic acid to form CS-g-bPEI (PBA) (CsPP). The inclusion of PBA is an important innovation that improves gene transfection and gene editing performance (FIGS. 48 and 49A-49D).

First, the hydrophobic interaction between PBA moieties stabilizes the polyplex and protects the encapsulated plasmids, especially under acidic conditions, such as in the stomach. Second, the PBA moiety, which has mucoadhesive properties, acts as a ligand to facilitate transport through the mucus layer. Third, after entering the target cells, the PBA enhances endosomal escape via hydrophobic interaction at late endosomal pH (pH 5.5), and subsequently binds to cytoplasmic ATP, which weakens the charged interactions of the polyplex and promo-ng the unpacking or release of the plasmid DNA in the cytosol. We have confirmed these features in an intracellular trafficking and fluorescence resonance energy transfer (FRET) study. Collectively, these features significantly enhance the gene delivery efficiency of the carrier, and the facilitated release in the cytosol is particularly advantageous for the delivery of Cas9 mRNA.

### Targeted Delivery to Lymph Nodes

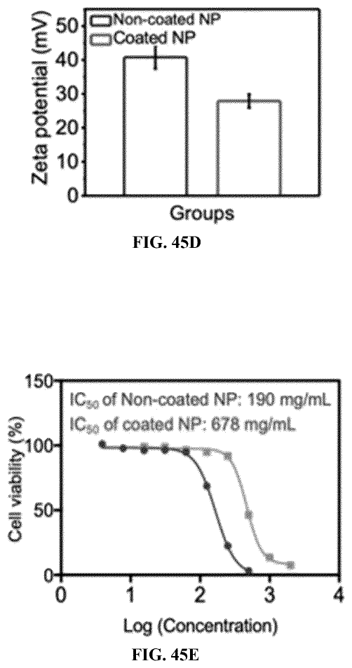

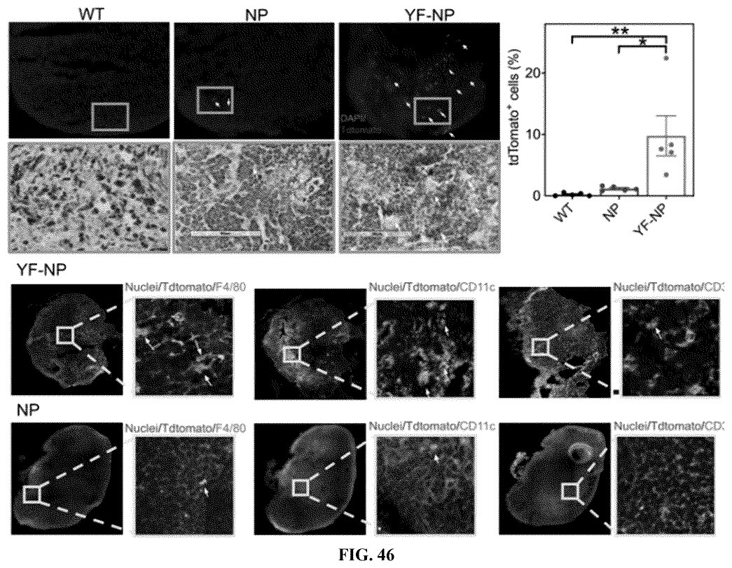

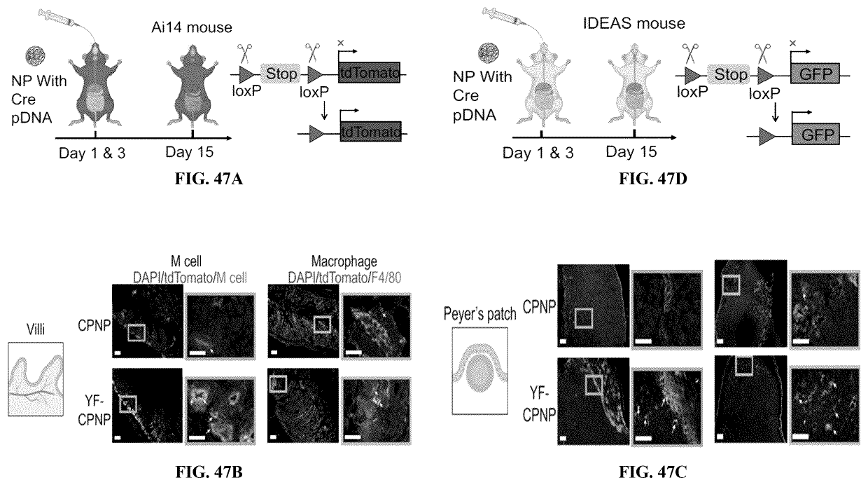

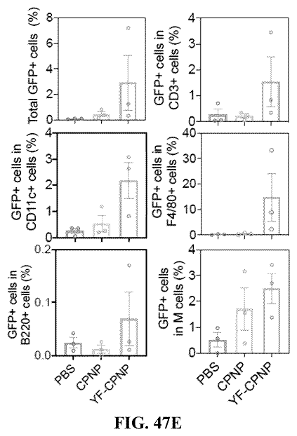

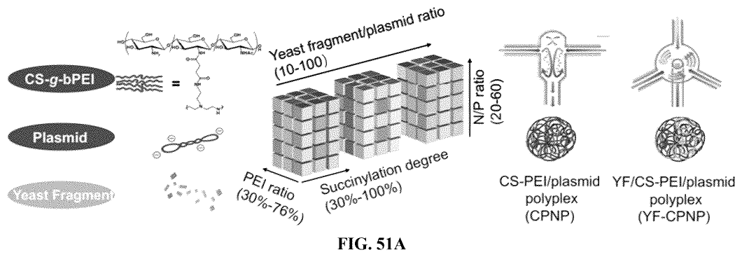

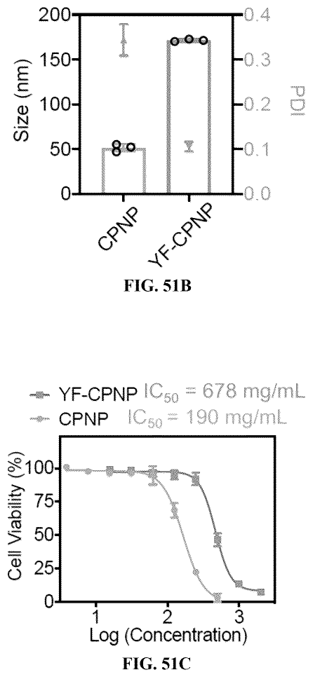

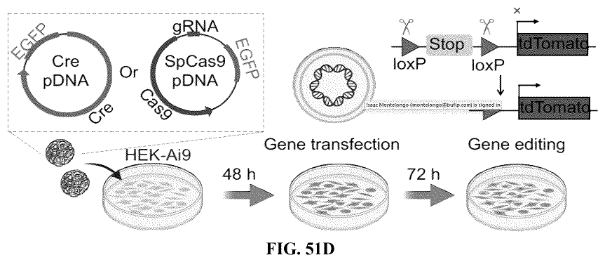

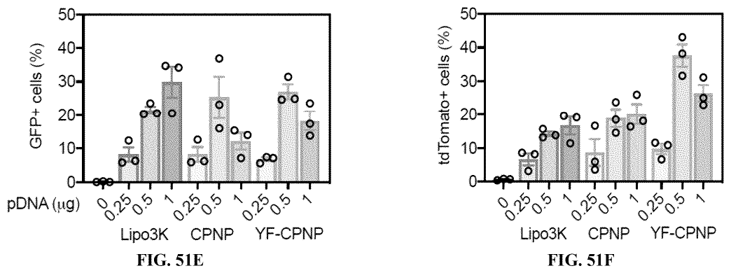

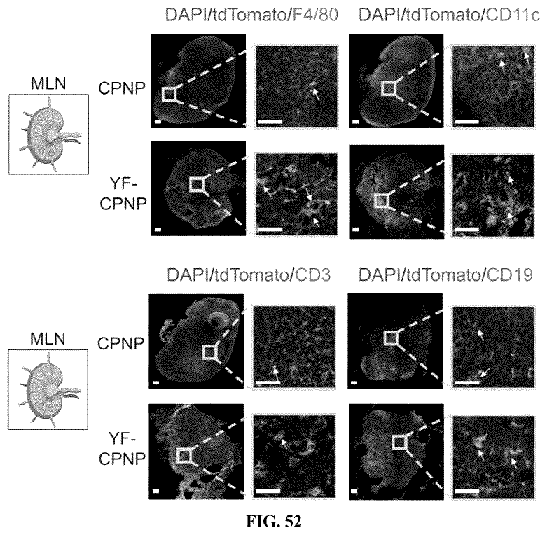

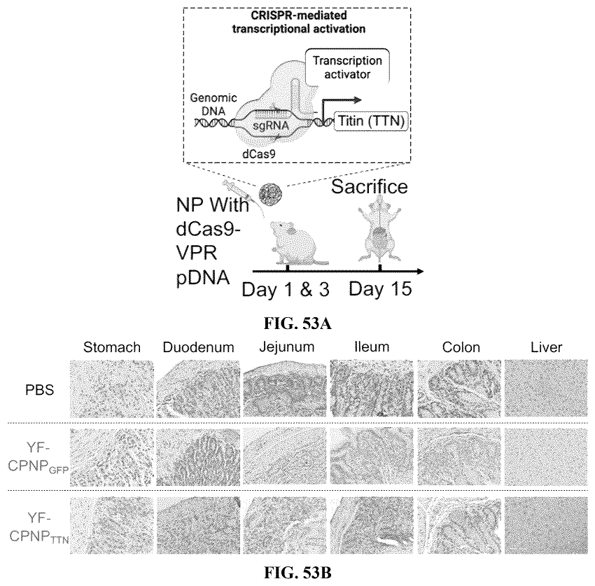

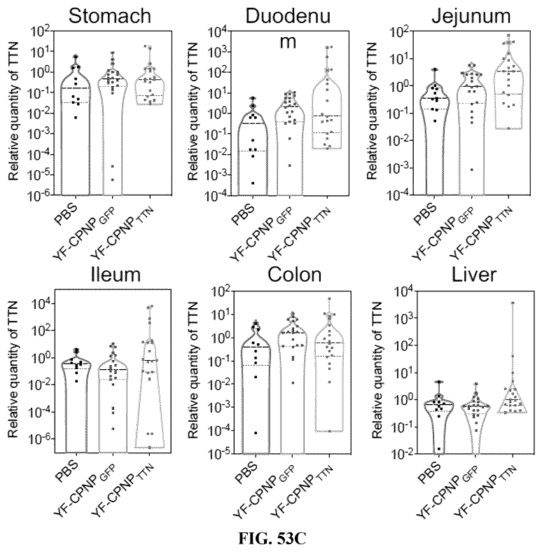

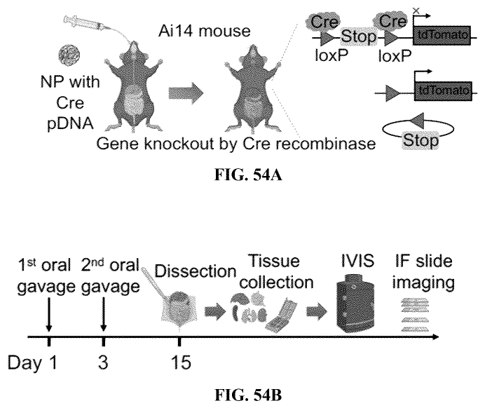

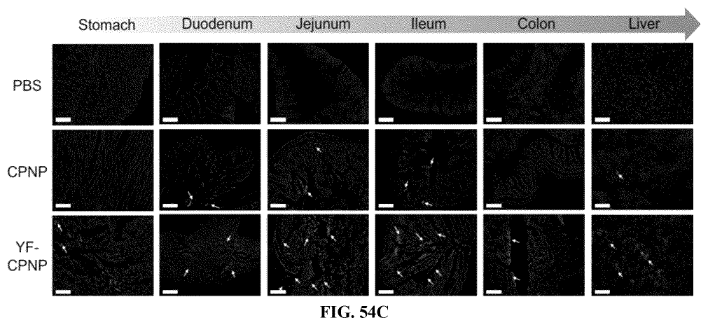

By incorporating yeast fragments into the polyplex (FIG. 51A), our oral nanoparticle platform YF-CPNP for gene editing plasmids can efficiently traverse tightly packed mucosal epithelium via intestinal microfold cells (M cells). Its subsequent endocytosis occurs in local and Peyer's patch macrophages, leading to a further accumulation in mesenteric lymph nodes (MLN) (FIGS. 47B-47C and 52). With these advantages, YF-CPNP can achieve gene editing in the macrophages, dendritic cells, T cells and B cells in the lymph nodes. This strategy not only prolongs gene editing within the local GI tract and gut-associated lymphoid tissues but also facilitates NP transport through the gut-associated lymphoid pathway to the systemic circulation, ultimately modulating the genome of distal organs such as the liver. This culminates in the manifestation of titin gene activation in the small intestine and liver through oral administration with YF-CPNP with dCas9 VPR plasmid (FIG. 53A).

Using the optimal CsPP polyplexes carrying the CRE/GFP plasmid, we showed that oral delivery to Ai14 mice (B₆.Cg-Gt(ROSA)26Sortm14(CAG-tdTomato)Hze/J, JAX Lab) resulted in detectable TdTomato signals in both small intestine and liver tissues at 48 hours post-administration using tissue clearing and Lightsheet imaging. This observation confirms the cleavage of the lox-stop-lox sequence, activating the tdTomato reporter gene via Cre recombinase. It serves as compelling evidence that the CsPP carrier can traverse the intestinal tight junction and initiate gene editing within just 48 hours following oral dosing.

### Targeted Delivery to Liver

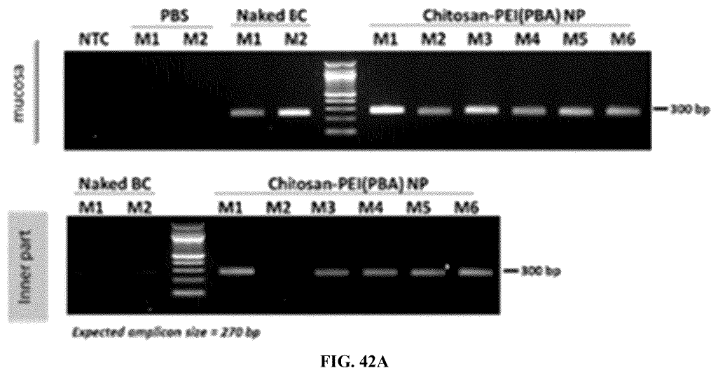

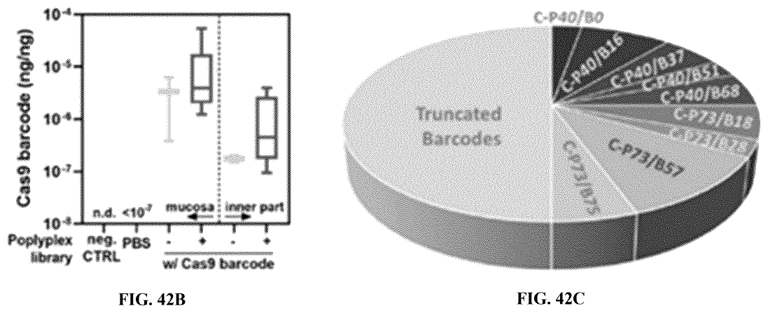

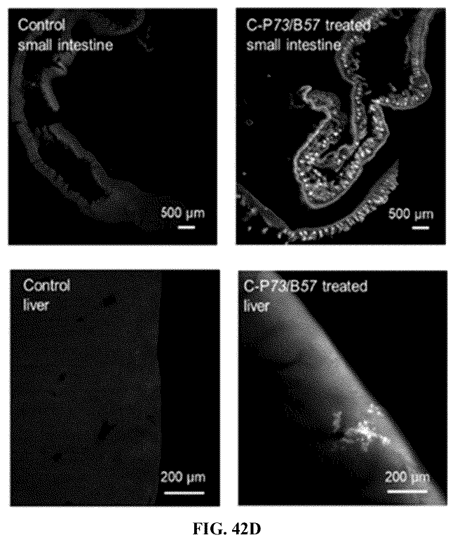

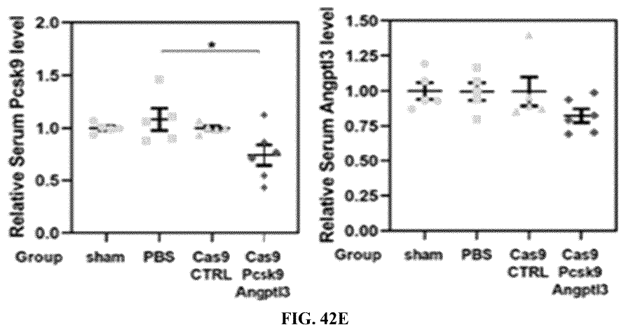

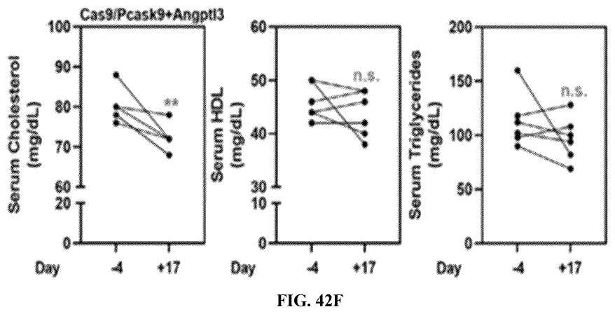

The feasibility of oral gene editing, targeting two well-characterized liver genes, PCSK9 and ANGPTL3, known to regulate blood low-density lipoprotein and triglyceride levels, respectively. Mice received oral CsPP gavages every other day for a total of three administrations. On Day 17, we collected mouse sera and liver tissues for analysis. Editing by the CsPP polyplexes led to a significant reduction in mouse serum PCSK9 levels, along with downstream lipid changes (FIG. 42). Importantly, no adverse effects were observed in blood clinical chemistry markers. While a similar reduction trend was observed for ANGPTL3 levels, the effect was moderate, likely due to the positioning of the Angptl3 guide's U6 expression cassette within the plasmid.

Coating the CsPP polyplexes with HepG2 cell membrane is expected to improve their delivery to the liver. Hepatocyte membranes carry specific proteins and receptors that are recognized by other liver cells, including Kupffer cells and endothelial cells lining the blood vessels in the liver. Hepatocytes also express galactose and asialoglycoprotein receptors on their surface. Therefore, coating the polyplex with HepG2 cell membrane fragments may enhance the nanoparticle's affinity for the liver.

Targeting ligands such as N-acetylgalactosamine (GalNac), or preferably triantennary N-Acetylgalactosamine, can also be conjugated to the CsPP surface to achieve hepatocyte-specific delivery. Another advantage of cell membrane coating is minimizing aggregation of the nanoparticles. Therefore, it is worth exploring whether the cell membrane coating can improve navigation across the gut epithelium and enhance liver gene editing efficiency.

### Targeted Delivery to the GI Tract

The feasibility of editing the GI tract by orally delivering the dCas9/VPR/EGFP/gRNA-TTN plasmid to activate the TTN gene is presented in this experiment. The presented results suggest the possibility of DNA editing with a higher efficiency because the dCas9/VPR/EGFP/gRNA-TTN pDNA is over 15 KB (nonviral transfection is significantly more efficient with smaller plasmids or mRNAs). The titin gene, TTN, encodes the protein titin, an exceptionally large protein (3,000 to 3,800 KDa molecular weight) found predominantly in muscle tissues, specifically in both skeletal and cardiac muscles. It acts as a molecular spring, providing elasticity to muscle fibers and contributing to their structural integrity.

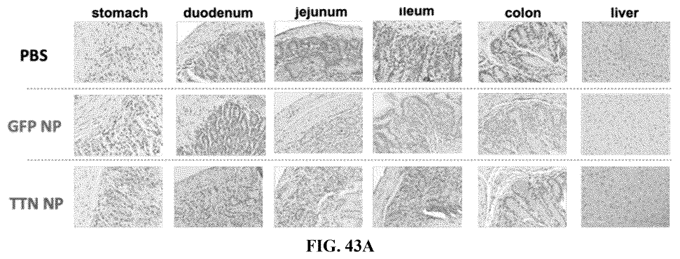

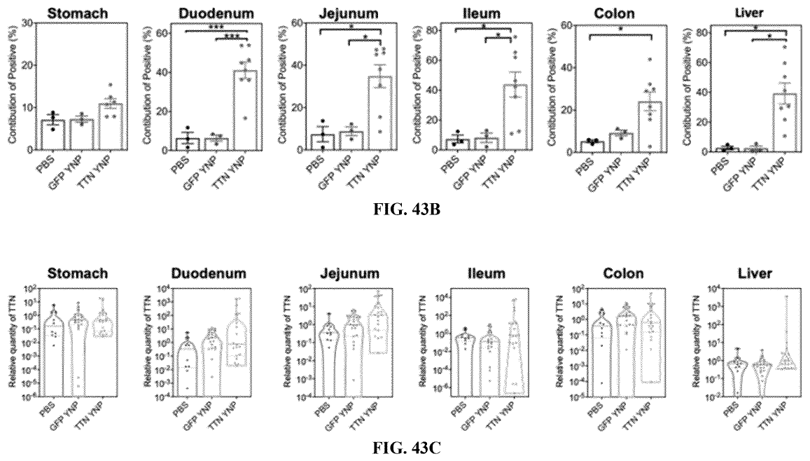

To achieve the TNN activation in the GI tract, we administered two oral gavages of 450 μg of pDNA each, at 0 and 48 hours. By day 15, immunohistochemical staining clearly revealed TNN protein expression in the colon, with positive results also observed in other segments of the GI tract (FIGS. 43A-43C and 53B-53C). Additionally, qPCR analysis confirmed elevated TNN gene expression in the treated group compared to the controls (FIG. 43C).

The Ai14 reporter mouse model will be used to further evaluate the GI-editing system and enhancements. For example, coating the polyplexes with Caco2 cell membranes is expected to improve localization within the GI tract and enhance gene editing efficiency. Other enhancements include the polyplexes with Eudragit S100 and FS30D polymers, known for their pH sensitivity to protect the oral formulation from degradation in the stomach and targeting the formulation the colon.

An exfoliome assay will be used longitudinal studies of GI tract gene editing efficiency. Studying gene changes in the GI tract has historically been challenging due to the difficulty of obtaining longitudinal samples. However, fecal matter contains a significant amount of host-derived materials, including DNA and RNA, due to the natural turnover of intestinal cells. This exfoliated cellular material, known as the exfoliome, contains valuable RNA signatures that can be used to monitor gene-c changes in the GI tract. Overcoming the challenge of background microbial and diet-associated nucleic acid molecules in stool samples. The non-invasive host gut transcriptome profiling method using unbiased multiplex amplification begins with extracting total RNA extracted from feces following by processing the extracted RNA sample with DNA digestion and reverse transcription to generate total cDNA. Multiplex PCR is used to amplify targeted genes with limited cycles, followed by the addition of Illumina adapters to yield amplicons for next-generation sequencing. This exfoliome analysis is effective in identifying gene modulation, including the expression of the TTN gene product, and verifying the presence of the dCas components.

### Targeted Delivery to the Mesenteric Lymph Node (MLN)

The mesenteric lymph nodes (MLNs) are sentinel hubs of enteral immunosurveillance and immune homeostasis. They play an important role in immune defense against pathogens in the drain the gastrointestinal (GI) tract, controlling infection and avoiding systemic inflammation. They are also critical to food allergy reactions and mucosal vaccination. Gene editing in the MLNs is a novel topic in the field of gene therapy but can potentially offer a way to modulate immune responses for therapeutic and prophylactic purposes. MLNs drain the GI tract by connecting to small clusters of lymphoid follicles spread out along the intestine, named Peyer's patches (PPs). Taking advantage of this connectivity, an oral delivery system that target the MLNs was developed. The disclosed formulation achieved gene editing in the MLNs.

The tested oral delivery system comprises nanoparticles formed from a cationic polymer named chitosan-grafted-branched polyetherimide (CS-g-bPEI) and an anionic plasmid DNA that encodes gene editing tools such as Cre-LoxP. Due to the electrostatic force, the polymer and the plasmid were attracted to each other and assembled into nanoparticles via a technique named flash nanocomplexation (FIG. 51A). In order to target the nanoparticles to the MLNs, we added a coating of yeast fragments (YFs) to the nanoparticles. The YFs are derived from Saccharomyces cerevisiae, aka baker's yeast. They contain β-glucans, a group of polysaccharides binding to the Dectin-1 receptors on the surface of microfold cells (M cells) in the gut epithelium that covers PPs and antigen present cells (APCs) in both PPs and MLNs. The overall delivery mechanism can be described as follows (FIG. 48): (1) the nanoparticles are administered orally and enter the GI tract; (2) the nanoparticles are uptaken by the M cells in the gut epithelium, relocated to the PP, and then transported to the MLNs nearby; (3) at the MLNs, the nanoparticles are endocytosed by APCs, releasing the plasmid DNA intracellularly, which leads to gene Cre-LoxP controlled gene editing.

Our preliminary results have validated both the delivery mechanism and the gene editing efficacy in MLNs. The nanoparticles were tested in Ai14 mice, a special reporter mouse strain that can highlight Cre-LoxP gene editing by tdTomato fluorescence (FIG. 49A). In order to detect the tdTomato signals in various organ segments on the cellular level, organs and tissues were harvested after IACUC-approved sacrifice, and the organ segments were imaged via confocal microscopy. A summary of the in vivo experiments is shown in FIG. 49B. Our results proved the importance of YFs in assisting PP mediated transportation to MLNs. By comparing the nanoparticles with and without YFs, we found that the presence of YFs significantly increases the uptake by the GI epithelium and ensures relocation to PPs and MLNs (FIGS. 49C, 49D; FIG. 46). Quantification of the tdTomato level showed ~10% of cells in MLNs had positive gene editing when the mice were treated with YF-containing nanoparticles, while in the YF-free samples, only 1% of cells in MLNs underwent gene editing (FIG. 46A). We also took a closer look into the type of cells involved in transporting YF-containing nanoparticles and had gene editing by immunohistochemistry staining. In the GI epithelium and the PPs, colocalization of tdTomato and the macrophage marker (F4/80) as well as the M cell markers indicated these two cell types facilitated nanoparticle uptake (FIG. 49D). In the MLNs, similar analysis proved high nanoparticle uptake by macrophages, dendritic cells, and T cells, using such markers as F4/80, CD11c, and CD3, respectively (FIG. 46B).

Overall, this invention achieves gene editing in the MLNs through an oral delivery route. It uses a non-viral carrier for gene delivery, which is safer and more translatable than the viral counterparts. The oral delivery method is noninvasive, which further strengthen its value in clinical translation.

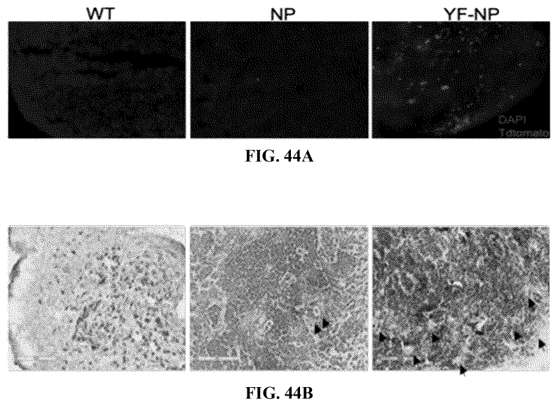

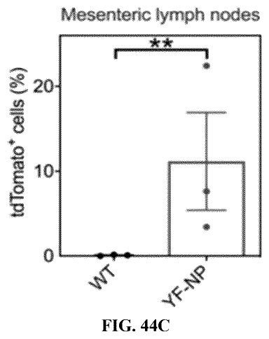

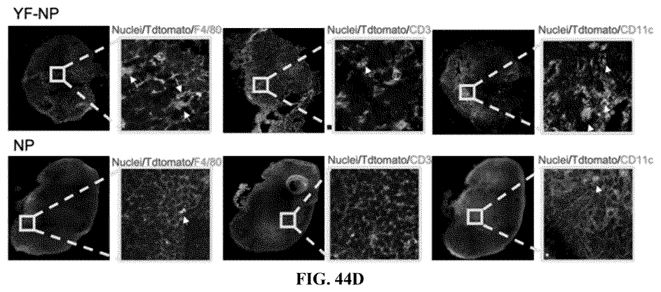

Gene editing in the MLNs is an emerging topic in the field of gene therapy and, considering the physiological function of MLNs, it holds great potentials in modulating immune responses for therapeutic and prophylactic purposes. β-glucans, a major component of the yeast cell wall, are recognized by the immune system, particularly through Dectin-1 receptors expressed on immune cells. Hypothesizing that beta-glucans would facilitate delivery to the intestinal lymphatic system, we explored the possibility of incorporating them into our CsP polyplexes for gene editing of the mesenteric lymph node (MLN). We extracted the cell membrane of budding yeast and used it to coat the CsP polyplexes carrying the CRE plasmid for oral delivery to Ai14 mice (FIG. 47). These yeast membrane fragments-coated CsP polyplexes are named YF-NP. Using a dosing regimen similar to that for GI tract editing, we observed substantial editing in MLN after two weeks, with approximately 10% of the cells edited (FIG. 44). CsP polyplexes lacking the yeast membrane coating exhibited significantly lower gene editing efficacy. Further characterization revealed that the majority of tdTomato-positive cells in the MLN were macrophages (F4/80 positive) and dendritic cells (CD11c positive) (FIG. 46). These cell types, known for their high dec-n-1 surface expression, exhibited enhanced uptake of YF-NP, resulting in markedly increased gene editing efficiency.

In summary, incorporating yeast fragments in CS-g-bPEI polyplexes enhances the delivery of oral gene editing. YF-CPNP demonstrates superior gene editing capabilities compared to CPNP, particularly excelling in M cells and macrophages throughout the GI tract and lymphoid tissues. Notably, it exhibits a distinct affinity for intestinal M cells, enhancing transepithelial absorption and subsequent accumulation in gut associated lymphoid tissues. Consequently, CP-YFNP facilitates superior gene editing in various immune cells within Peyer's patches and MLN (FIG. 47). The enhanced accumulation of YF-CPNP in the lymphatic pathway suggests potential systemic circulation to distant organs such as the liver. Furthermore, we successfully achieved oral gene activation of TTN using YF-CPNP with the dCas9 VPR plasmid, both in the GI tract and liver. Thus, oral administration of YF-CPNP enables gene modulation in the GI tract, extending its effects to the liver. Overall, our findings highlight the promise of an effective, orally-administered, nonviral gene modulation system for targeted editing in the GI tract and gut-associated lymphoid tissues, as well as systemic gene activation, offering a patient-friendly, non-invasive approach

### Example 7. Evaluation of Oral Formulation for Administering siRNA

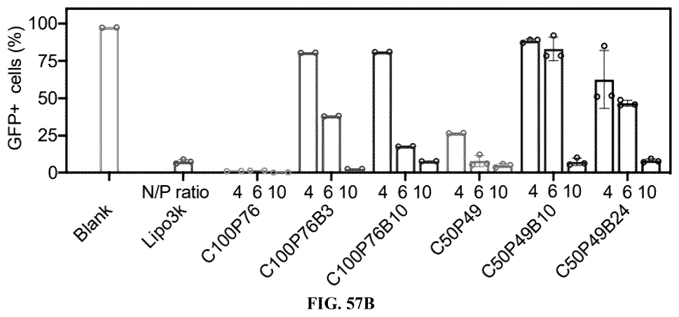

A series of CP or CPB polyplexes containing GFP siRNA was prepared for in vitro validation of siRNA gene silencing efficiency in the U2OS-EGFP cell line. Both CP and CPB polyplexes efficiently silenced the GFP gene in a dose-dependent manner in U2OS-EGFP cell lines (FIGS. 57A and 57B). The incorporation of PBA reduces the charge density of the polymer, thereby weakening the electronic interaction between the polymer and siRNA, leading to a decrease in GFP silencing efficiency. However, with an increase in the PBA ratio, the advantages of PBA in endosomal escape and siRNA cytosolic release enhanced the GFP silencing efficiency. Thus, CPB is a promising carrier for siRNA delivery.

### Example 8. Manufacturing of Polyplexes and Coated Polyplexes

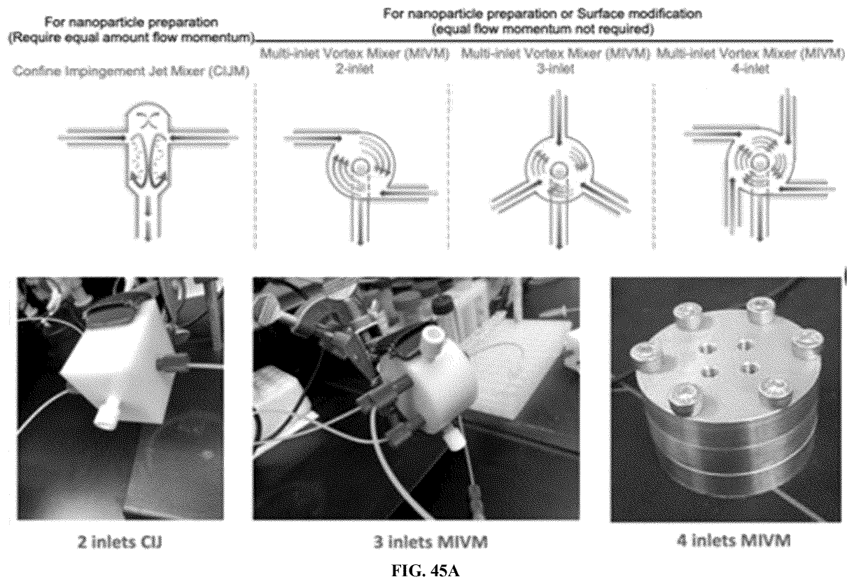

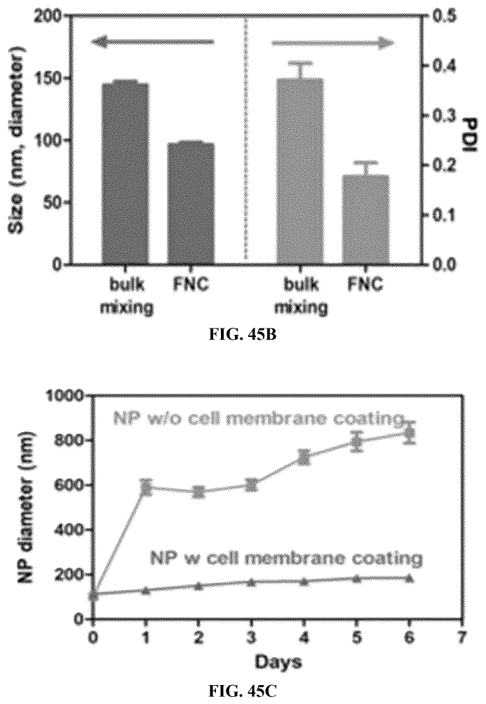

Flash nanocomplexation (FNC), which involves rapid mixing of macromolecules in confined impinging jet mixers, facilitates the self-assembly of polyplexes driven by electrostatic interactions. We have developed various FNC devices to achieve scalable polyplex production (FIG. 45). The polyplexes generated by FNC had better uniformity in size, improved colloidal stability, lower batch-to-batch variation, and higher transfection performance than those produced by conventional bulk-mixing technique. Furthermore, we demonstrated that FNC can be used to coat a variety of nanoparticle cores (including the coated therapeutic agents described herein) with various types of cell membrane to produce nanoparticles with small size and narrow size distribution. The principles are the same as the microfluidic processes used to produce lipid nanoparticles for COVID-19 vaccine formulations, therefore, scalable for translation.

### REFERENCES CITED AND INCORPORATED BY REFERENCE

Bento, D.; Jesus, S.; Lebre, F.; Gonçalves, T.; Borges, O. Chitosan Plus Compound 48/80: Formulation and Preliminary Evaluation as a Hepatitis B Vaccine Adjuvant. Pharmaceutics 2019, 11 (2), 72, DOI: 10.3390/pharmaceutics11020072.
Brannigan, R. P.; Khutoryanskiy, V. V. Progress and Current Trends in the Synthesis of Novel Polymers with Enhanced Mucoadhesive Properties. Macromol. Biosci. 2019, 19 (10), 1900194, DOI: 10.1002/mabi.201900194.
Brinkman, E. K.; Chen, T.; Amendola, M.; van Steensel, B. Easy quantitative assessment of genome editing by sequence trace decomposition. Nucleic Acids Res. 2014, 42 (22), e168-e168. DOI: 10.1093/nar/gku936.
Chadwick, A. C.; Evitt, N. H.; Lv, W.; Musunuru, K. Reduced Blood Lipid Levels With In Vivo CRISPR-Cas9 Base Editing of ANGPTL3. Circulation 2018, 137 (9), 975-977, DOI: 10.1161/CIRCULATIONAHA.117.031335.
Chuan, D.; Jin, T.; Fan, R.; Zhou, L.; Guo, G. Chitosan for gene delivery: Methods for improvement and applications. Adv. Colloid Interface Sci. 2019, 268, 25-38, DOI: 10.1016/j.cis.2019.03.007.
Cohen, J. C.; Boerwinkle, E.; Mosley, T. H.; Hobbs, H. H. Sequence Variations in PCSK9, Low L D L, and Protection against Coronary Heart Disease. N. Engl. J. Med. 2006, 354 (12), 1264-1272, DOI: 10.1056/NEJMoa054013.
Dahlman, J. E.; Kauffman, K. J.; Xing, Y.; Shaw, T. E.; Mir, F. F.; Dlott, C. C.; Langer, R.; Anderson, D. G.; Wang, E. T. Barcoded nanoparticles for high throughput in vivo discovery of targeted therapeutics. Proc. Natl. Acad. Sci. U.S.A 2017, 114 (8), 2060, DOI: 10.1073/pnas. 1620874114.
DeSalle, R.; Goldstein, P. Review and Interpretation of Trends in DNA Barcoding. Front. Ecol. Evol. 2019, 7, 302, DOI: 10.3389/fevo.2019.00302.
Ellis, G. A.; Palte, M. J.; Raines, R. T. Boronate-Mediated Biologic Delivery. J. Am. Chem. Soc. 2012, 134 (8), 3631-3634, DOI: 10.1021/ja210719s.
Farris, E.; Heck, K.; Lampe, A. T.; Brown, D. M.; Ramer-Tait, A. E.; Pannier, A. K. Oral non-viral gene delivery for applications in DNA vaccination and gene therapy. Curr. Opin. Biomed. Eng. 2018, 7, 51-57, DOI: 10.1016/j.cobme.2018.09.003.
Ferraretto, A.; Bottani, M.; De Luca, P.; Cornaghi, L.; Arnaboldi, F.; Maggioni, M.; Fiorilli, A.; Donetti, E. Morphofunctional properties of a differentiated Caco2/HT-29 co-culture as an in vitro model of human intestinal epithelium. Biosci. Rep. 2018, 38 (2), BSR20171497, DOI: 10.1042/BSR20171497.
Friedl, H.; Dünnhaupt, S.; Hintzen, F.; Waldner, C.; Parikh, S.; Pearson, J. P.; Wilcox, M. D.; Bernkop-Schnürch, A. Development and Evaluation of a Novel Mucus Diffusion Test System Approved by Self-Nanoemulsifying Drug Delivery Systems. J. Pharm. Sci. 2013, 102 (12), 4406-4413, DOI: 10.1002/jps.23757.
Godbey, W. T.; Wu, K. K.; Mikos, A. G. Size matters: Molecular weight affects the efficiency of poly(ethylenimine) as a gene delivery vehicle. J. Biomed. Mater. Res. 1999, 45 (3), 268-275, DOI: 10.1002/(SICI) 1097-4636 (19990605) 45: 3<268::AID-JBM15>3.0.CO; 2-Q.
Gong, J.; Wang, H.-X.; Lao, Y.-H.; Hu, H.; Vatan, N.; Guo, J.; Ho, T.-C.; Huang, D.; Li, M.; Shao, D.; Leong, K. W. A Versatile Nonviral Delivery System for Multiplex Gene-Editing in the Liver. Adv. Mater. 2020, 32 (46), 2003537, DOI: 10.1002/adma.202003537.
Gorman, M. W.; Feigl, E. O.; Buffington, C. W. Human Plasma ATP Concentration. Clin. Chem. 2007, 53 (2), 318-325, DOI: 10.1373/clinchem.2006.076364.
Horváth, G.; Petrás, M.; Szentesi, G.; Fábián, Á.; Park, J. W.; Vereb, G.; Szöllösi, J. Selecting the right fluorophores and flow cytometer for fluorescence resonance energy transfer measurements. Cytometry A 2005, 65A (2), 148-157, DOI: 10.1002/cyto.a.20142.
Jiang, Y.; Dong, S.; Qin, G.; Liu, L.; Zhao, H. Oxidation and ATP dual-responsive block copolymer containing tertiary sulfoniums: self-assembly, protein complexation and triggered release. Polym. Chem. 2021, 12 (8), 1125-1135, DOI: 10.1039/DOPY01622E.
Kim, J.; Lee, Y. M.; Kim, H.; Park, D.; Kim, J.; Kim, W. J. Phenylboronic acid-sugar grafted polymer architecture as a dual stimuli-responsive gene carrier for targeted anti-angiogenic tumor therapy. Biomaterials 2016, 75, 102-111, DOI: 10.1016/j.biomaterials.2015.10.022.
Kim, J.; Vaughan, H. J.; Zamboni, C. G.; Sunshine, J. C.; Green, J. J. High-throughput evaluation of polymeric nanoparticles for tissue-targeted gene expression using barcoded plasmid DNA. J. Controlled Release 2021, 337, 105-116, DOI: 10.1016/j.jconrel.2021.05.047.
Lang, X.; Wang, T.; Sun, M.; Chen, X.; Liu, Y. Advances and applications of chitosan-based nanomaterials as oral delivery carriers: A review. Int. J. Biol. Macromol. 2020, 154, 433-445, DOI: 10.1016/j.ijbiomac.2020.03.148.
Lao, Y.-H.; Li, M.; Gao, M. A.; Shao, D.; Chi, C.-W.; Huang, D.; Chakraborty, S.; Ho, T.-C.; Jiang, W.; Wang, H.-X.; Wang, S.; Leong, K. W. HPV Oncogene Manipulation Using Nonvirally Delivered CRISPR/Cas9 or Natronobacterium gregoryi Argonaute. Adv. Sci. 2018, 5 (7), 1700540, DOI: 10.1002/advs.201700540.
Li, C.; Huang, Z.; Liu, Z.; Ci, L.; Liu, Z.; Liu, Y.; Yan, X.; Lu, W. Sulfonate-modified phenylboronic acid-rich nanoparticles as a novel mucoadhesive drug delivery system for vaginal administration of protein therapeutics: improved stability, mucin-dependent release and effective intravaginal placement. Int. J. Nanomed. 2016, 11, 5917-5930, DOI: 10.2147/IJN.S113658.
Li, F.; Wang, L.; Jin, X.-M.; Yan, C.-H.; Jiang, S.; Shen, X.-M. The immunologic effect of TGF-beta1 chitosan nanoparticle plasmids on ovalbumin-induced allergic BALB/c mice. Immunobiology 2009, 214 (2), 87-99, DOI: 10.1016/j.imbio.2008.07.007.
Lin, P.-Y.; Chiu, Y.-L.; Huang, J.-H.; Chuang, E.-Y.; Mi, F.-L.; Lin, K.-J.; Juang, J.-H.; Sung, H.-W.; Leong, K. W. Oral Nonviral Gene Delivery for Chronic Protein Replacement Therapy. Adv. Sci. 2018, 5 (8), 1701079, DOI: 10.1002/advs.201701079.
Liu, C.; Kou, Y.; Zhang, X.; Cheng, H.; Chen, X.; Mao, S. Strategies and industrial perspectives to improve oral absorption of biological macromolecules. Expert Opin. Drug Delivery 2018, 15 (3), 223-233, DOI: 10.1080/17425247.2017.1395853.
Lokugamage, M. P.; Sago, C. D.; Dahlman, J. E. Testing thousands of nanoparticles in vivo using DNA barcodes. Curr. Opin. Biomed. Eng. 2018, 7, 1-8, DOI: 10.1016/j.cobme.2018.08.001.
Lorand, J. P.; Edwards, J. O. Polyol Complexes and Structure of the Benzeneboronate Ion. J. Org. Chem. 1959, 24 (6), 769-774, DOI: 10.1021/jo01088a011.
Maddalo, D.; Manchado, E.; Concepcion, C. P.; Bonetti, C.; Vidigal, J. A.; Han, Y.-C.; Ogrodowski, P.; Crippa, A.; Rekhtman, N.; de Stanchina, E.; et al. In vivo engineering of oncogenic chromosomal rearrangements with the CRISPR/Cas9 system. Nature 2014, 516 (7531), 423-427. DOI: 10.1038/nature13902.
Mosialou, I; Shikhel, S; Liu, J M; Maurizi, A; Luo, N; He, Z; Huang, Y; Zong, H; Friedman, RA; Barasch, J; Lanzano, P; Deng, L; Leibel, R L; Rubin, M; Nickolas, T; Chung, W; Zeltser, LM; Williams, KW; Pessin, JE; Kousteni S. MC4R-dependent suppression of appetite by bone-derived lipocalin 2. Nature. 2017 Mar. 16; 543 (7645): 385-390. doi: 10.1038/nature21697.
Mosialou, I; Shikhel, S; Luo, N; Petropoulou, PI; Panitsas, K; Bisikirska, B; Rothman, NJ; Tenta, R; Cariou, B; Wargny, M; Sornay-Rendu, E; Nickolas, T; Rubin, M; Confavreux, CB; Kousteni, S. Lipocalin-2 counteracts metabolic dysregulation in obesity and diabetes. J Exp Med. 2020 Oct. 5; 217 (10): e20191261. doi: 10.1084/jem.20191261.
Matsumoto, Y.; Itaka, K.; Yamasoba, T.; Kataoka, K. Intranuclear fluorescence resonance energy transfer analysis of plasmid DNA decondensation from nonviral gene carriers. J. Gene Med. 2009, 11 (7), 615-623, DOI: 10.1002/jgm.1338.
Mintz, R. L.; Lao, Y.-H.; Chi, C.-W.; He, S.; Li, M.; Quek, C. H.; Shao, D.; Chen, B.; Han, J.; Wang, S.; Leong, K. W. CRISPR/Cas9-mediated mutagenesis to validate the synergy between PARP1 inhibition and chemotherapy in BRCA1-mutated breast cancer cells. Bioeng. Transl. Med. 2020, 5 (1), e10152 DOI: 10.1002/btm2.10152.
Mudie, D. M.; Amidon, G. L.; Amidon, G. E. Physiological Parameters for Oral Delivery and in Vitro Testing. Mol. Pharmaceutics 2010, 7 (5), 1388-1405, DOI: 10.1021/mp100149j.
Naito, M.; Ishii, T.; Matsumoto, A.; Miyata, K.; Miyahara, Y.; Kataoka, K. A Phenylboronate-Functionalized Polyion Complex Micelle for ATP-Triggered Release of siRNA. Angew. Chem., Int. Ed. 2012, 51 (43), 10751-10755, DOI: 10.1002/anie.201203360.
Naito, M.; Yoshinaga, N.; Ishii, T.; Matsumoto, A.; Miyahara, Y.; Miyata, K.; Kataoka, K. Enhanced Intracellular Delivery of siRNA by Controlling ATP-Responsivity of Phenylboronic Acid-Functionalized Polyion Complex Micelles. Macromol. Biosci. 2018, 18 (1), 1700357, DOI: 10.1002/mabi.201700357.
O'Driscoll, C. M.; Bernkop-Schnürch, A.; Friedl, J. D.; Préat, V.; Jannin, V. Oral delivery of non-viral nucleic acid-based therapeutics-do we have the guts for this?. Eur. J. Pharm. Sci. 2019, 133, 190-204, DOI: 10.1016/j.ejps.2019.03.027.
Patil, S. J.; Naik, B. J. Carrier Based Oral Nano Drug Delivery Framework: A Review. Curr. Nanomater. 2018, 3 (2), 75-85, DOI: 10.2174/2405461503666180703094241.
Paunovska, K.; Sago, C. D.; Monaco, C. M.; Hudson, W. H.; Castro, M. G.; Rudoltz, T. G.; Kalathoor, S.; Vanover, D. A.; Santangelo, P. J.; Ahmed, R.; Bryksin, A. V.; Dahlman, J. E. A Direct Comparison of in Vitro and in Vivo Nucleic Acid Delivery Mediated by Hundreds of Nanoparticles Reveals a Weak Correlation. Nano Lett. 2018, 18 (3), 2148-2157, DOI: 10.1021/acs.nanolett.8b00432.
Petropoulou, PI; Mosialou, I; Shikhel, S; Hao, L; Panitsas, K; Bisikirska, B; Luo, N; Bahna, F; Kim, J; Carberry, P; Zanderigo, F; Simpson, N; Bakalian, M; Kassir, S; Shapiro, L; Underwood, MD; May, CM; Soligapuram, Sai K K; Jorgensen, MJ; Confavreux, CB; Shapses, S; Laferrère, B; Mintz, A; Mann, JJ; Rubin, M; Kousteni, S. Lipocalin-2 is an anorexigenic signal in primates. Elife. 2020 Nov. 24; 9: e58949. doi: 10.7554/eLife.58949.
Prosperi-Porta, G.; Kedzior, S.; Muirhead, B.; Sheardown, H. Phenylboronic-Acid-Based Polymeric Micelles for Mucoadhesive Anterior Segment Ocular Drug Delivery. Biomacromolecules 2016, 17 (4), 1449-1457, DOI: 10.1021/acs.biomac.6b00054.
Qi, Y.; Yu, T.; Xu, J.; Wan, P.; Ma, Y.; Zhu, J.; Li, Y.; Gong, H.; Luo, Q.; Zhu, D. FDISCO: Advanced solvent-based clearing method for imaging whole organs. Sci. Adv. 2019, 5 (1), eaau8355. DOI: doi: 10.1126/sciadv.aau8355.
Ran, F. A.; Hsu, P. D.; Wright, J.; Agarwala, V.; Scott, D. A.; Zhang, F. Genome engineering using the CRISPR-Cas9 system. Nat. Protoc. 2013, 8 (11), 2281-2308. DOI: 10.1038/nprot.2013.143.
Schneider, S.; Lenz, D.; Holzer, M.; Palme, K.; Süss, R. Intracellular FRET analysis of lipid/DNA complexes using flow cytometry and fluorescence imaging techniques. J. Controlled Release 2010, 145 (3), 289-296, DOI: 10.1016/j.jconrel.2010.04.016.
Sezgin-Bayindir, Z.; Antep, M. N.; Yuksel, N. Development and Characterization of Mixed Niosomes for Oral Delivery Using Candesartan Cilexetil as a Model Poorly Water-Soluble Drug. AAPS PharmSciTech 2015, 16 (1), 108-117. DOI: 10.1208/s12249-014-0213-9.
Springsteen, G.; Wang, B. A detailed examination of boronic acid-diol complexation. Tetrahedron 2002, 58 (26), 5291-5300, DOI: 10.1016/S0040-4020 (02) 00489-1.
Steinberg, D.; Witztum, J. L. Inhibition of PCSK9: a powerful weapon for achieving ideal LDL cholesterol levels. Proc. Natl. Acad. Sci. U.S.A 2009, 106 (24), 9546-9547, DOI: 10.1073/pnas.0904560106.
Stubelius, A.; Lee, S.; Almutairi, A. The Chemistry of Boronic Acids in Nanomaterials for Drug Delivery. Acc. Chem. Res. 2019, 52 (11), 3108-3119, DOI: 10.1021/acs.accounts.9b00292 [ACS Full Text ACS Full Text], [CAS], Google Scholar
Sugawara, Y.; Yutani, M.; Amatsu, S.; Matsumura, T.; Fujinaga, Y. Functional Dissection of the Clostridium botulinum Type B Hemagglutinin Complex: Identification of the Carbohydrate and E-Cadherin Binding Sites. PLOS ONE 2014, 9 (10), e111170. DOI: 10.1371/journal.pone.0111170.
Tong, S.; Moyo, B.; Lee, C. M.; Leong, K.; Bao, G. Engineered materials for in vivo delivery of genome-editing machinery. Nat. Rev. Mater. 2019, 4 (11), 726-737, DOI: 10.1038/s41578-019-0145-9.
Van Duin, M.; Peters, J. A.; Kieboom, A. P. G.; Van Bekkum, H. Studies on borate esters 1: The ph dependence of the stability of esters of boric acid and borate in aqueous medium as studied by 11B NMR. Tetrahedron 1984, 40 (15), 2901-2911, DOI: 10.1016/S0040-4020 (01) 91300-6.
von Harpe, A.; Petersen, H.; Li, Y.; Kissel, T. Characterization of commercially available and synthesized polyethylenimines for gene delivery. J. Control. Release 2000, 69 (2), 309-322. DOI: 10.1016/S0168-3659 (00) 00317-5.
Wan, P.; Li, Y.; Zhu, J.; Xu, J.; Liu, X.; Yu, T.; Zhu, D. FDISCO+: a clearing method for robust fluorescence preservation of cleared samples. Neurophotonics 2021, 8 (3), 035007, DOI: 10.1117/1.NPh.8.3.035007.
Wang, Q.; Yang, Y.; Liu, D.; Ji, Y.; Gao, X.; Yin, J.; Yao, W. Cytosolic Protein Delivery for Intracellular Antigen Targeting Using Supercharged Polypeptide Delivery Platform. Nano Lett. 2021, 21 (14), 6022-6030, DOI: 10.1021/acs.nanolett. 1c01190.
Yaari, Z.; da Silva, D.; Zinger, A.; Goldman, E.; Kajal, A.; Tshuva, R.; Barak, E.; Dahan, N.; Hershkovitz, D.; Goldfeder, M.; Roitman, J. S.; Schroeder, A. Theranostic barcoded nanoparticles for personalized cancer medicine. Nat. Commun. 2016, 7 (1), 13325, DOI: 10.1038/ncomms13325.
Yan, J.; Springsteen, G.; Deeter, S.; Wang, B. The relationship among pKa, pH, and binding constants in the interactions between boronic acids and diols—it is not as simple as it appears. Tetrahedron 2004, 60 (49), 11205-11209, DOI: 10.1016/j.tet.2004.08.051.
Ye, L.; Liu, H.; Fei, X.; Ma, D.; He, X.; Tang, Q.; Zhao, X.; Zou, H.; Chen, X.; Kong, X.; Liu, P. Enhanced endosomal escape of dendrigraft poly-L-lysine polymers for the efficient gene therapy of breast cancer. Nano Res. 2022, 15, 1135-1144, DOI: 10.1007/s12274-021-3616-4.
Yeh, T.-H.; Hsu, L.-W.; Tseng, M. T.; Lee, P.-L.; Sonjae, K.; Ho, Y.-C.; Sung, H.-W. Mechanism and consequence of chitosan-mediated reversible epithelial tight junction opening. Biomaterials 2011, 32 (26), 6164-6173, DOI: 10.1016/j.biomaterials.2011.03.056.
Yin, H.; Kauffman, K. J.; Anderson, D. G. Delivery technologies for genome editing. Nat. Rev. Drug Discovery 2017, 16 (6), 387-399, DOI: 10.1038/nrd.2016.280.
Yoshinaga, N.; Ishii, T.; Naito, M.; Endo, T.; Uchida, S.; Cabral, H.; Osada, K.; Kataoka, K. Polyplex Micelles with Phenylboronate/Gluconamide Cross-Linking in the Core Exerting Promoted Gene Transfection through Spatiotemporal Responsivity to Intracellular pH and ATP Concentration. J. Am. Chem. Soc. 2017, 139 (51), 18567-18575. DOI: 10.1021/jacs.7b08816.
Yoshinaga, N.; Uchida, S.; Dirisala, A.; Naito, M.; Koji, K.; Osada, K.; Cabral, H.; Kataoka, K. Bridging mRNA and Polycation Using RNA Oligonucleotide Derivatives Improves the Robustness of Polyplex Micelles for Efficient mRNA Delivery. Adv. Health. Mater. 2022, 11, 2102016, DOI: 10.1002/adhm.202102016.
Yoshinaga, N.; Uchida, S.; Dirisala, A.; Naito, M.; Osada, K.; Cabral, H.; Kataoka, K. mRNA loading into ATP-responsive polyplex micelles with optimal density of phenylboronate ester crosslinking to balance robustness in the biological milieu and intracellular translational efficiency. J. Controlled Release 2021, 330, 317-328, DOI: 10.1016/j.jconrel.2020.12.033.
Yoshinaga, N; Zhou, J K; Xu, C; Quek, C H; Zhu, Y; Tang, D; Hung, L Y; Najjar, S A; Shiu, C Y A; Margolis, K G; Lao, Y H; Leong, K W. Phenylboronic Acid-Functionalized Polyplexes Tailored to Oral CRISPR Delivery. Nano Lett. 2023 Feb. 8; 23 (3): 757-764. doi: 10.1021/acs.nanolett.2c02306.
Zhang, J.; Tang, C.; Yin, C. Galactosylated trimethyl chitosan-cysteine nanoparticles loaded with Map4k4 siRNA for targeting activated macrophages. Biomaterials 2013, 34 (14), 3667-3677, DOI: 10.1016/j.biomaterials.2013.01.079.
Zhang, P.; Wagner, E. History of Polymeric Gene Delivery Systems. Top. Curr. Chem. 2017, 375 (2), 26, DOI: 10.1007/s41061-017-0112-0.
Schoeffel U, Pelz K, Häring R U, Amberg R, Schandl R, Urbaschek R, von Specht B U, Farthmann E H. Inflammatory consequences of the translocation of bacteria and endotoxin to mesenteric lymph nodes. Am J Surg. 2000 July; 180 (1): 65-72.
Ma Y, Yang X, Chatterjee V, Wu M H, Yuan S Y. The Gut-Lung Axis in Systemic Inflammation. Role of Mesenteric Lymph as a Conduit. Am J Respir Cell Mol Biol. 2021 January; 64 (1): 19-28.
Nakajima-Adachi H, Kikuchi A, Fujimura Y, Shibahara K, Makino T, Goseki-Sone M, Kihara-Fujioka M, Nochi T, Kurashima Y, Igarashi O, Yamamoto M, Kunisawa J, Toda M, Kaminogawa S, Sato R, Kiyono H, Hachimura S. Peyer's patches and mesenteric lymph nodes cooperatively promote enteropathy in a mouse model of food allergy. PLOS One. 2014 Oct. 7; 9 (10): e107492.
Brandtzaeg P. Mucosal immunity: induction, dissemination, and effector functions. Scand J Immunol. 2009 December; 70 (6): 505-15.
Dennehy K M, Brown G D. The role of the beta-glucan receptor Dectin-1 in control of fungal infection. J Leukoc Biol. 2007 August; 82 (2): 253-8.
Ali A and Ahmed S, A review on chitosan and its nanocomposites in drug delivery. International Journal of Biological Macromolecules, (2018). 109: p. 273-286.
Zhao Y, Li Y, Wang F, Gan X, Zheng T, Chen M, Wei L, Chen J, and Yu C, CES1-Triggered Liver-Specific Cargo Release of CRISPR/Cas9 Elements by Cationic Triadic Copolymeric Nanoparticles Targeting Gene Editing of PCSK9 for Hyperlipidemia Amelioration. Adv Sci (Weinh), (2023). 10 (19): p. e2300502.
Tilson S G, Morell C M, Lenaerts A S, Park S B, Hu Z, Jenkins B, Koulman A, Liang T J, and Vallier L, Modeling PNPLA3-Associated NAFLD Using Human-Induced Pluripotent Stem Cells. Hepatology, (2021). 74 (6): p. 2998-3017.
Kim U, Kim N, and Shin H Y, Modeling Non-Alcoholic Fatty Liver Disease (NAFLD) Using “Good-Fit” Genome-Editing Tools. Cells, (2020). 9 (12).
Rutten M G S, Derks T G J, Huijkman N C A, Bos T, Kloosterhuis N J, van de Kolk K, Wolters J C, Koster M H, Bongiovanni L, Thomas R E, de Bruin A, van de Sluis B, and Oosterveer M H, Modeling Phenotypic Heterogeneity of Glycogen Storage Disease Type 1a Liver Disease in Mice by Somatic CRISPR/CRISPR-associated protein 9-Mediated Gene Editing. Hepatology, (2021). 74 (5): p. 2491-2507.
Lee Y K, Du W, Nie Y, Diaz B, Sultana N, Kitamoto T, Leibel R L, Accili D, and Belvedere S, Single-agent FOXO1 inhibition normalizes glycemia and induces gut beta-like cells in streptozotocin-diabetic mice. Mol Metab, (2022). 66: p. 101618.
Du W, Wang J, Kuo T, Wang L, McKimpson W M, Son J, Watanabe H, Kitamoto T, Lee Y, Creusot R J, Ratner L E, McCune K, Chen Y W, Grubbs B H, Thornton M E, Fan J, Sultana N, Diaz B S, Balasubramanian I, Gao N, Belvedere S, and Accili D, Pharmacological conversion of gut epithelial cells into insulin-producing cells lowers glycemia in diabetic animals. J Clin Invest, (2022). 132 (24).
Mojibian M, Glavas M M, and Kieffer T J, Engineering the gut for insulin replacement to treat diabetes. J Diabetes Investig, (2016). 7 Suppl 1 (Suppl 1): p. 87-93
Sledz M, Wojciechowska A, Zagozdzon R, and Kaleta B, In Situ Programming of CAR-T Cells: A Pressing Need in Modern Immunotherapy. Arch Immunol Ther Exp (Warsz), (2023). 71 (1): p. 18.
Qin Y T, Li Y P, He X W, Wang X, Li W Y, and Zhang Y K, Biomaterials promote in vivo generation and immunotherapy of CART cells. Front Immunol, (2023). 14: p. 1165576.
Chen X, Shi T, Yang C, Chen F, He X, Zhang K, Hu H, Cai L, Leong K W, and Shao D, Scalable biomimetic SARS-COV2 nanovaccines with robust protective immune responses. Signal Transduct Target Ther, (2022). 7 (1): p. 96.
Hu H, Yang C, Zhang F, Li M, Tu Z, Mu L, DawulieV J, Lao Y H, Xiao Z, Yan H, Sun W, Shao D, and Leong K W, A Versatile and Robust Platform for the Scalable Manufacture of Biomimetic Nanovaccines. Adv Sci (Weinh), (2021). 8 (15): p. 2002020.
He Z Y, Santos J L, Tian H K, Huang H H, Hu Y Z, Liu L X, Leong K W, Chen Y M, and Mao H Q, Scalable fabrication of size controlled chitosan nanoparticles for oral delivery of insulin. Biomaterials, (2017). 130: p. 28-41.
He Z Y, Hu Y Z, Nie T Q, Tang H Y, Zhu J C, Chen K T, Liu L X, Leong K W, Chen Y M, and Mao H Q, Size-controlled lipid nanoparticle production using turbulent mixing to enhance oral DNA delivery. Acta Biomaterialia, (2018). 81: p. 195-207.
Nie T Q, He Z Y, Zhou Y, Zhu J C, Chen K T, Liu L X, Leong K, Mao H Q, and Chen Y M, Surface Coating Approach to Overcome Mucosal Entrapment of DNA Nanoparticles for Oral Gene Delivery of Glucagon-like Peptide 1. Acs Applied Materials & Interfaces, (2019). 11 (33): p. 29593-29603.
Hu H, Yang C, Li M, Shao D, Mao H Q, and Leong K W, Flash Technology-Based Self-Assembly in Nanoformulation: From Fabrication to Biomedical Applications. Mater Today (Kidlington), (2021). 42: p. 99-116.
Santos J L, Ren Y, Vandermark J, Archang M M, Williford J M, Liu H W, Lee J, Wang T H, and Mao H Q, Continuous Production of Discrete Plasmid DNA-Polycation Nanoparticles Using Flash Nanocomplexation. Small, (2016). 12 (45): p. 6214-6222.
Angsan Vkul P, Thamphiwatana S, Zhang Q, Spiekermann K, Zhuang J, Fang R H, Gao W, Obonyo M, and Zhang L, Coating nanoparticles with gastric epithelial cell membrane for targeted antibiotic delivery against Helicobacter pylori infection. Adv Ther (Weinh), (2018). 1 (2).
Gao W and Zhang L, Coating nanoparticles with cell membranes for targeted drug delivery. J Drug Target, (2015). 23 (7-8): p. 619-26.
Wang D, Wang S, Zhou Z, Bai D, Zhang Q, Ai X, Gao W, and Zhang L, White Blood Cell Membrane-Coated Nanoparticles: Recent Development and Medical Applications. Adv Healthc Mater, (2022). 11 (7): p. e2101349
Das S, Pectin based multi-particulate carriers for colon-specific delivery of therapeutic agents. Int J Pharm, (2021). 605: p. 120814.
Dong, Y., Siegwart, D. J. and Anderson, D. G., 2019. Strategies, design, and chemistry in siRNA delivery systems. Advanced drug delivery reviews, 144, pp. 133-147.
Kanasty, R., Dorkin, J. R., Vegas, A. and Anderson, D., 2013. Delivery materials for siRNA therapeutics. Nature materials, 12 (11), pp. 967-977.

We claim:

1. An oral formulation comprising:
a polymer comprising phenyl boric acid (PBA)-functionalized chitosan grafted with branched polyethyleneimine (PEI); and
a therapeutic agent, wherein the therapeutic agent is encapsulated by the polymer.

2. The oral formulation of claim 1, wherein the PBA-functionalized chitosan grafted with PEI is a block copolymer.

3. The oral formulation of claim 1, wherein the PBA is 4-Carboxy-3-fluorophenylboronic acid.

4. The oral formulation of claim 1, wherein the chitosan has a molecular weight of about 1,000 Da to about 40,000 Da or has a molecular weight of about 15,000 Da.

5. The oral formulation of claim 1, wherein the branched PEI has a molecular weight of about 600 Da to about 10,000 Da or has a molecular weight of about 800 Da to about 2,000 Da.

6. The oral formulation of claim 1, wherein the polymer is of the formula CS-PEI_{X%}(PBA)_{Y%} where CS is a chitosan residue, PBA is a phenyl boric acid residue, and PEI is a branched polyethyleneimine polymer residue, and where x % is the PEI grafting ratio and y % is the PBA introduction ratio, and wherein x % is 35 to 75%, y % is 5 to 59%.

7. The oral formulation of claim 1, wherein the amount of therapeutic agent in the oral formulation is 1.79 to 21.4% relative to the amount of polymer.

8. The oral formulation of claim 1, wherein the loading capacity of the polymer is around 17.8% to 22%.

9. The oral formulation of claim 1, wherein the therapeutic agent is a protein or a peptide therapeutic.

10. The oral formulation of claim 9, wherein the protein or peptide therapeutic is Lipocalin 2 or osteocalcin.

11. The oral formulation of claim 1, wherein the therapeutic agent provides gene therapy.

12. The oral formulation of claim 11, wherein the therapeutic agent is a plasmid or siRNA, or the therapeutic agent comprises a CRISPR/Cas system.

13. The oral formulation of claim 1, further comprising eukaryotic cell membrane fragment, wherein the eukaryotic cell membrane fragment coats the encapsulated therapeutic agent.

14. An oral formulation comprising:
a polymer comprising chitosan grafted with branched polyethyleneimine (PEI);
a therapeutic agent comprising a nucleic acid sequence, wherein the therapeutic agent is encapsulated by the polymer to produce an encapsulated therapeutic agent; and
a eukaryotic cell membrane fragment, wherein the eukaryotic cell membrane fragment coats the encapsulated therapeutic agent.

15. The oral formulation of claim 14, wherein the eukaryotic cell membrane fragment is a yeast membrane fragment.

16. A method of producing an orally bioavailable form of a therapeutic agent comprising encapsulating a therapeutic agent with a polymer comprising phenyl boric acid (PBA)-functionalized chitosan grafted with a branched polyethyleneimine (PEI) to produce an encapsulated therapeutic agent, wherein the therapeutic agent is selected from: Lipocalin 2, osteocalcin, and a gene therapy product.

17. The method of claim 16, wherein the therapeutic agent is a gene therapy product and the method produces an encapsulated gene therapy product, the method further comprises coating the encapsulated gene therapy product with a eukaryotic cell membrane fragment.

18. A method of reducing weight in a subject comprising orally administering to the subject the oral formulation of claim 9.

19. A method of administering gene therapy to a subject comprising orally administering to the subject the oral formulation of claim 11.

20. The method of claim 19, wherein the oral formulation further comprises an eukaryotic cell membrane fragment, wherein the eukaryotic cell membrane fragment coats the polymer that encapsulates the therapeutic product.
